SEQUENCE LISTING (19 sequences)

SEQ ID NO: 1 - DNA, 10207 bp, Staphylococcus aureus
  Designated as SA-24
aagcttatggacctattttaggtatattgattagttggcttggattaatttctggaacatttacagtctatttgatctgtaaacgattggtgaacactgagaggatgcagcgaattaaacaacgtactgctgttcaacgcttgattagttttattgatcgccaaggattaatcccattgtttattttactttgttttccttttacgccaaatacattaataaattttgtagcgagtctatctcatattagacctaaatattatttcattgttttggcatcatcaaagttagtttcaacaattattttaggttatttaggtaaggaaattactacaattttaacgcatcctttaagagggatattaatgttagttgtgttggttgtattttggattgttggaaaaaagttagaacagcattttatgggatcgaaaaaggagtgacatcgtgaaaaaagttgtaaaatatttgatttcattgatacttgctattatcattgtactgttcgtacaaacttttgtaatagttggtcatgtcattccgaataatgatatgtcaccaacccttaacaaagggacgtgttattgtaaataaaattaaagttacatttaatcaattgaataatggtgatatcattacatataggcgtggtaacgagatatatactagtcgaattattgccaaacctggtcaatcaatggcgtttcgtcagggacaattataccgtgatgaccgaccggttgacgcatcttatgccaagaacagaaaaattaaagattttagtttgcgcaattttaaagaattagatggagatattataccgcctaacaattttgttgtgctaaatgatcatgataacaatcagcatgattctagacaatttggtttaattgataaaaaggatattattggtaatataagtttgagatattatcctttttcaaaatggacgattcagttcaaatcttaaaaagaggtgtcaaaattgaaaaaagaattattggaatggattatttcaattgcagtcgcttttgtcattttatttatagtaggtaaatttattgttacaccatatacaattaaaggtgaatcaatggatccaactttgaaagatggcgagcgagtagctgtaaacattattggatataaaacaggtggtttggaaaaaggtaatgtagttgtcttccatgcaaacaaaaatgatgactatgttaaacgtgtcatcggtgttcctggtgataaagtagaatataaaaatgatacattatatgtcaatggtaaaaaacaagatgaaccatatttaaactataatttaaaacataaacaaggtgattacattactgggactttccaagttaaagatttaccgaatgcgaatcctaaatcaaatgtcattccaaaaggtaaatatttagttcttggagataatcgtgaagtaagtaaagatagccgtgcgtttggcctcattgatgaagaccaaattgttggtaaagtttcatttagattctggccatttagtgaatttaaacataatttcaatcctgaaaatactaaaaattaatatgaaacaaatacaacatcgtttgtcggttttaatactgataaacgatgttttattttgttagtaccacaataaaagctaagttcgaaatgaacttataataaatcaatcacaatcactttgtgttaaaatatgtgtcaaaggaagtgagggtttgtcatgacattacatgcttatttaggtagagcgggaacaggtaagtctacgaaaatgttgaccgaaataaaacaaaaaatgaaagcagatccgcttggagatccaatcattttaattgcgccaactcaaagtacatttcaattagaacaagcctttgtcaatgatccggaattaaatggtagtttaagaacagaagtgttgcattttgaacgattaagtcatcgtattttccaagaagttggtagttatagcgaacaaaagttatctaaagctgcaacggaaatgatgatttataacattgttcaagaacaacaaaagtatttaaaactttatcaatcacaagcaaaatattatgggtttagtgaaaaattaacagaacaaattcaagattttaaaaaatatgcagtaacgcctgaacatttagaacactttattgctgataaaaatatgcaaactcgaactaaaaataagttagaggatattgctttaatataccgtgagttcgaacaacgcattcaaaacgagtttattactggtgaggattcattacaatattttattgattgtatgccgaaatcagagtggctaaaacgtgctgatatatatattgatggttttcacaacttttcaacgattgagtatttaataatcaaaggattaattaaatatgcgagagtgtcacaattatattgacgacagatggtaaccacgatcaatttagttttttagaaaaccatcggaagtgttacgacatattgaagaaatagcaaatgaactcaatatttctattgaacgtcaatatttcaaccaattatatcgcttcaataatcaagatttaaagcatcttgaacaagaatttgatgtacttcaaatcaatcgagtggcatgtcaaggtcatatcaatattttagaatctgcgactatgagagaggaaataaatgaaattgcgcgacgtatcatcgttgatattcgtgataagcaattacgatatcaagatattgcaattttatatcgtgacgagtcttatgcttatttatttgattccatattaccgctttataatattccttataacattgatacaaagcgttcgatgacacatcatccggtcatggaaatgattcgttcattgattgaagttattcaatctaattggcaagtgaatccaatgctacgcttattgaagactgatgtgttaacggcatcatatctaaaaagtgcatacttagttgatttacttgaaaattttgtacttgaacgtggtatatacggtaaacgttggttagatgatgagctatttaatgtcgaacattttagcaaaatggggcgtaaagcgcataaactgaccgaagatgaacgtaacacatttgaacaagtcgttaagttaaagaaagatgtcattgataaaattttacattttgaaaagcaaatgtcacaagcggaaactgtaaaagactttgcaactgctttttatgaaagtatggaatatttcgaactgccaaatcaattgatgacagagcgagatgaacttgatttaaatggtaatcatgaaaaggcggaggaaattgatcaaatatggaatggcttaattcaaatccttgacgacttagttctagtatttggagatgaaccaatgtcgatggaacgtttcttagaagtatttgatattggtttagaacaattagaatttgtcatgattccacaaacattagatcaagttagtattggtacgatggatttggctaaagtcgacaataagcaacatgtttacttagttggaatgaacgacggcaccatgccacaaccagtaactgcatcaagtttaattactgatgaagaaaagaaatattttgaacaacaagcaaatgtagagttgagtcctacatcagatattttacagatggatgaagcatttgtttgctatgttgctatgactagagctaagggagatgttacattttcttacagtctaatgggatcaagtggtgatgataaggagatcagcccatttttaaatcaaattcaatcattgttcaaccaattggaaattactaacattcctcaataccatgaagttaacccattgtcactaatgcaacatgctaagcaaaccaaaattacattatttgaagcattgcgtgcttggttagatgatgaaattgtggctgatagttggttagatgcttatcaagtaattagagatagcgatcatttaaatcaaggtttagattatttaatgtcagcattaacgtttgacaatgaaactgtaaaattaggtgaaacgttgtctaaagatttatatggtaaggaaatcaatgccagtgtatctcgttttgaaggttatcaacaatgcccatttaaacactatgcttcacatggtctgaaactaaatgaacgaacgaaatatgaacttcaaaactttgatttaggtgatattttccattccgttttaaaatatatatctgaacgtattaatggcgattttaaacaattagacctgaaaaaaataagacaattaacgaatgaagcattggaagaaattttacctaaagttcagtttaatttattaaattcttcagcttactatcgttatttatcaagacgcattggcgctattgtagaaacaacactaagcgcattaaaatatcaaggcacgtattcaaagtttatgccaaaacattttgagacaagttttagaaggaaaccaagaaccaaatgtacgaattaattgcacaaacattaacgacaactcaaggtattccaattaatattagagggcaaattgaccgtatcgatacgtatacaaagaatgatacaagttttgttaatatcattgactataaatcctctgaaggtagtgcgacacttgatttaacgaaagtatattatggtatgcaaatgcaaatgatgacatacatggatatcgttttacaaaataaacaacgccttggattaacagatattgtgaaaccaggtggattattatacttccatgtacatgaacctagaattaaatttaaatcatggtctgatattgatgaagataaactagaacaagatttaattaaaaagtttaagctgagtggtttagtgaatgcagaccaaactgttattgatgcattggatattcgtttagaacctaaattcacttcagatattgtaccagttggtttgaataaagatggctctttgagtaaacgaggcagccaagtggcagatgaagcaacaatttataaattcattcagcataacaaagagaattttatagaaacagcttcaaatattatggatggacatactgaagtgcaccattaaagtacaaacaaaaattgccatgtgctttttgtagttatcaatcggtatgtcatgtagatggcatgattgatagtaagcgatatcgaactgtagatgaaacaataaatccaattgaagcaattcaaaatattaacattaatgatgaatttgggggtgagtaatagatgacaattccagagaaaccacaaggcgtgatttggactgacgcgcaatggcaaagtatttacgcaactggacaagatgtacttgttgcagccgcggcaggttcaggtaaaacagctgtactagttgagcgtattatccaaaagattttacgtgatggcattgatgtcgatcgacttttagtcgtaacgtttacaaacttaagcgcacgtgaaatgaagcatcgtgtagaccaacgtattcaagaggcatcgattgctgatcctgcaaatgcacacttgaaaaaccaacgcatcaaaattcatcaagcacaaatatctacacttcatagtttttgcttgaaattaattcaacagcattatgatgtattaaatattgacccgaactttagaacaagcagtgaagctgaaaatattttattattagaacaaacgatagatgaggtcatagaacaacattacgatatccttgatcctgcttttattgaattaacagaacaattgtcttcagatagaagtgatgatcagtttcgaatgattattaaacaattgtatttctttagcgttgcaaatccaaatcctacaaattggttggatcaattggtgacaccatacgaagaagaagcacaacaagcgcaacttattcaactactaacagacttatctaaagtatttatcacagctgcctatgatgctttaaataaggcgtatgatttgtttagtatgatggatggcgtcgataaacatttagctgttatagaagatgaacgacgtttaatggggcgtgttttagaaggtggttttattgatataccttatttaactgatcacgaatttggcgcgcgtttgcctaatgtaacagcgaaaattaaagaagcaaatgaaatgatggtcgatgccttagaagatgctaaacttcagtataaaaaatataaatcattaattgataaagtgaaaaatgattacttttcaagagaagctgatgatttgaaagctgatatgcaacaattggcgccacgagtaaagtaccttgcgcgtattgtgaaagatgttatgtcagaattcaatcgaaaaaagcgtagcaaaaatattctggatttttctgattatgaacaatttgcattacaaattttaactaatgaggatggttcgccttcagaaattgccgaatcataccgtcaacactttcaagaaatattggtcgatgagtatcaagatacgaaccgggttcaagagaaaatactatcttgcatcaaaacgggtgatgaacataatggtaatttatttatggttggagatgttaagcaatccatttataaatttagacaagctgatccaagtttatttattgaaaagtatcaacgctttactatagatggagatggcactggacgtcgaattgatttgtcgcaaaactccgttctcgaaaagaagtactgtcaacgactaactatatatcaaacatatgatggatgaacaagtcggtgaagtaaaatatgatgaagcggcacagttgtattatggtgcaccatatgatgaatcggaccatccagtaaacttaaaagtgcttgttgaagcggatcaagaacatagtgatttaactggtagtgaacaagaagcgcattttatagtagaacaagttaaagatatcttagaacatcaaaaagtttatgatatgaaaacaggaagctatagaagtgcgacatacaaagatatcgttattctagaacgcagctttggacaagctcgcaatttacaacaagcctttaaaaatgaagatattccattccatgtgaatagtcgtgaaggttactttgaacaaacagaagtccgcttagtattatcatttttaagagcgatagataatccattacaagatatttatttagttgggttaatgcgctccgttatatatcagttcaaagaagacgaattagctcaaattagaatattgagtcaaatgatgactacttctatcaatcgattgtaaattacattaatgacgaagcagcagatgctattttagttgataaattaaaaatgtttttatcagatattcaaagttaccaacaatatagtaaagatcatccggtgtatcagttaattgataaattttataatgatcattatgttattcaatactttagtggacttattggtggacgtggacgacgtgcaaacctttatggtttatttaataaagctatcgagtttgagaattcaagttttagaggtttatatcaatttattcgttttatcgatgaattgattgaaagaggcaaagattttggtgaggaaaatgtagttggtccaaacgataatgttgttagaatgatgacaattcatagtagtaaaggtctagagtttccatttgtcatttattctggattgtcaaaagattttaataaacgtgatttgaaacaaccagttattttaaatcagcaatttggtctcggaatggattattttgatgtggataaagaaatggcatttccatctttagcttcggttgcatataaagctgttgccgaaaaagaacttgtgtcagaagaaatgcgattagtctatgtagcattaacaagagcgaaagaacaactttatttaattggtagagtgaaaaattgataaatcgttactagaactagagcaattgtctatttctggtgagcacattgctgtcaatgaacgattaacttcaccaaatccgttccatcttatttatagtattttatctaaacatcaatctgcgtcaattccagatgatttaaaatttgaaaaagatatagcacaagttgaagatagtagtcgtccgaatgtaaatatttcaattatatactttgaagatgtgtctacagaaaccattttagataataatgaatatcgttcggttaatcaattagaaactatgcaaaatggtaatgaggatgttaaagcacaaattaaacaccaacttgattatcaatatccatatgtaaatgatactaaaaagccatccaaaacaatctgtttctgaattgaaaaggcaatatgaaagaagaaagtggcacaagttacgaacgagtaagacaatatcgtatcggttttcaacgtatgaacgacctaaatttctaagtgaacaaggtaaacgaaaaagcgaattgaaattggtacgttaatgcatacagtgatgcaacatttaccattcaaaaaagaacgcatatctgaagttgagttacatcagtatatcgatggattaatcgataaacatattatcgaagcagatgcgaaaaaagatatccgtatggatgaaataatgacattatcaatagtgagtatattcgattattgctgaagcagagcaagtttatcgtgaattaccgtttgtagttaaccaagcattagttgaccaattgccacaaggagacgaagacgtctcaattattcaaggtatgattgacttaatctttgttaaagatggtgtgcattattttgtagactataaaaccgatgcatttaatcgtcgccgtgggatgacagatgaagaaattggtacacaattaaaaaataaatataagatacagatgaaatattatcaaaatacgcttcaaacgatacttaataaagaagttaaaggttatttatacttcttcaaatttggtacattgcaactgtagtattttgattttcaaaagaataaaaaataatttcgattaagtgcaaagtccttgtagcagaatgaacacaactcattttcaaaattgtcttacttatttatttgttatttgataacgaaaaaagttataatgtgaattaagataaagatgaggagttgagaatgaatgaaattcttatcattcaagtataatgacaaaacttcatatggcgttaaagtaaaacgcgaagatgctgtatgggatttaacacaagtatttgctgactttgcagaaggagatttccatcctaaaacattgttagctggtttacaacaaaatcatactttagattttcaagaacaagtacgtaaagcagttgtagcagcagaagatagcggcaaagctgaagactataaaatttcatttaatgacattgaattcttaccaccagtaacacctccgaataatgtgattgcttttggtagaaattacaaagatcatgcgaacgaattaaatcatgaagtagaaaaattatatgtatttacaaaagcagcgtcatctttaacaggagataatgcaacaattccaaatcataaagatattactgatcaattagattatgaaggtgaattaggtattgttattggtaagtctggtgaaaagattccaaaagcattagctttagattatgtttacggctatacaattattaacgatatcactgatcgcaaagcacaaagtgaacaagatcaagcatttttatcaaaaagtttaactggcggttgcccaatgggtccttatatcgttactaaagacgaactaccattacctgaaaatgtaaatattgttacaaaagttaacaatgaaattagacaagatggtaacactggcgaaatgattcttaaaattgatgaattaatagaagaaatttcaaaatatgttgcactactaccgggagattattattgcaactggtacaccagctggcgttggtgcaggtatgcaaccacctaaatttttacaaccaggtgatgaagttaaagtgactattgataatattggaacgctgacaacttatatcgctaaataattatcatttaaaaagctaaccaggtctttatatagattggttagttttttcttgcttttctaaaaaggtgttaaagataaattatttataatgttaccattttgagatgaaagtgaaatattgatattaagaagtagttgattattttacagcagattcacaatattctaataagggcaatgcaaatgtcatgttcttcctctcaaatatagaagtgtggtagaatatatattcgtgtataatcaaatctagattaaattacaagcaagtgggtattaatcccaagaagctt

SEQ ID NO: 2 - DNA, 2082 bp, Staphylococcus aureus
  Designated as SA-36
aagctttctaatctatcgttaatgatttgctttaaaattgggtcgaagttaattgaaggtgtgaagtgtatatctgtattaataaccatgtcattcatttgctgcttcactttgttaacaagtcttccgtcatataaaaataatggtacgacaatcaatttttgataccgtttcgagatgctttctaaatcatgtgtaaaactaatctctccatatagcgttctcgcataagtaggtttattaatctgcaaatgttgagcgcatatttgtaactcttcgtgtgccttagtaaaatttccattaatattgccgtgtgcaacaaccataactccaacttgttgttcgtcacctgctaatgcgtcacaaatacgttgttcaattaatcgtctcattaaaggatgtgtgccaagtggctcgcttacttctacctttatgtctggataccgtcgtttcatttcatgaacgatattcggtatatccttgagataatgcattgcactaaagattagcaatggtacaattttaaaatggtcaaccccactttgaatcaacgtcgtcattaccgtctctaaatcctgatgctcactttctaaaaacgcaatatcatagtgatgtatatcatcttttactaattcagaaataaatgcttctaacgcttgattctgtcgtccgtgcctcatgccatgtgcaacaatgatattcccattcacatttaccaaccctttcacacgtattgtataccaaatcattttgtttttgtgaaaagaatcacattataatgtaaaatcagggaattccctgatgcctgtagtcatgcatattccttatacattttccctttttgttaaatcaaaaaaagcgaccgatatatgaatccctactcaacatttatttgagcaagcatcaatatatcggtcgcttgtagtgtatattattatcttaaaatggtggttggcctaatattgtttcgtcaaagcgctcgggtatcaatactttgcgcatgatcacacctaaatcgccatcatcattttcatgttcgctgtatatttcataacctcttttttcataaattttaagtaaccacggatgcaatcttgcagatgtacctaaagtaactgccgctgactttaacgtatctcgcaaaaatgctcttcaacataagtaagtaattggctaccatagcctttcccttcatactcaggatttgtcgcaaaccaccagacaaaaggatagcccgaaatacttttcacacttccccaaggatatctaaccgtaatcgtagatataatttcatcatcaattgtcatgacaaatgtagtatttttatctatattttctttaacagcatctaaattagcattaactgaaggccaatcaatacctagttctcttagaggcgtaaatgcttcatgcatgagttgttgcaatttttctgcatcttgttcacttgcgagtcgaatcatcgtttttgtcatattaatccccactcttttttaaatgatttaaccatattttatttttaaaataaatatccatcaaagtgtatcaataaatttatcacatgtcagaaagtatgcttcatctgaatacaccaatactctcatgaaacttattaaaaattactctctcaacgtaaaaaaaccattcaaattcatgaatggtttggaagaatgattcattgttacgctatttaatcactacatcttaattattgttgctctaaacgattacgcttaccatttaagaaagcataaacgagacctacaaaaataccgccaccgacaaagttacctaagaaagcaaaaacgatattttttaaaacatgtaaccatgaaactgcatcaaggttaaagaataccatacctgcatatagacctgcattgaacacaacgtgctcatatcccatgtatacaaagaccacgacaccacaagctatgaagaatgcctttgttaagccgcctttgaattgcatagagatgaaaataccaatattaataaagaagttacagaaaataccttttgtaaaaatattcaaccatgttgaatcaacagtctttttctgaactaaagctgttaaagctt

SEQ ID NO: 3 - DNA, 2885 bp, Staphylococcus aureus
  Designated as SA-77
aagcttttgattaatttgggctttaaagtattcccaattataattcttcatgattttcttattggatttcgaatttggtttcatgcattgttgcctcaaagaacatgctgaacagtcatcgcattcatatagcttgaagtcacgtttaaaaccatatctatcattacggtatgcatatcttttaaaacctattcttttgttattaggacatataaattcatcattaagttcgtcatatttccaattttgagtgttaaaaatgtcacttttaaactttctagttttatctttaataaacatgccatacgtaataagtggcgttttattaaaacatctataatagccatatagttttgctcactatcataactgcatcagctacattaactctggtaataccgaggatttgaatcattgttaaaaatggaattaaagttctagtatctgttggggtttgaaataggtcataggataaaaaaattgagaatttgtcgctatttgtaaattgtatcctggcttaagttggccatttttcatatggtcttccttcattctcataaaagttgcatcatgatcagcccagaaagctatttctatctttaagaatccatttttgttcttcatatttattttttctttcggaataatcatcaaatttctttttgaacttcttaatctcagttcttttttacgggtctgttttctaatttgagcactcttcgttctaaatagaatgatttaaatcttcgatttcttttatctaaatgactaccaattaaatctatttcttctcgtgattttgaatacttttcttccacacaaatgtatatctattggcattagcttctacttatgtaccatcaataaaaattgaattattatcaataagattttgctttaaacattgactatggaactgaataaataaagattcaattaacgcatcagtattaggattcactctaaaacgattaatagttttataagaaggtgtttgatcttgagctaaccacatcattcgaatactgtcatgaagtaatttctctattctacgaccagaaaatacagattgagtatatgcatataagatgatttttaacatcatttttggatgataggatgttgcgccacgatgatgtctgaattcatcgaattcgctatcaggtatcgtttcaacaatttcatttacatatcgcgaaatatcattttaaggaattctaacagaagtttctattggtagtgtaagttgggcaaagtgtcttatttttttaaagtatgtaaaagtaaaattacatgttaatacgtagtattaatggcgagactcctgagggagcagtgccagtcgaagaccgaggctgagacggcaccctaggaaagcgaagcattcaatacgaagtattgtataaatagagaacagcagtaagatattttctaattgaaaattatcttactgctgtttttttagggatttatgtcccagcctgttttattttcgactagtttggagaatttattgacattcacattatttaaacggcaacaaagattgttttattttgataggcattatatggtgttaaaaaatttgcatgaaaattaaaaaatgcttcgttcaggaaggtgtcgtaatttacctatttgctgaatgaagcattttatttttaaatatgatagccaatataacaagctataaatccaatgatgaattgtaaaagtgaataattgagaaaaaggttaatatcaaattttggtgtcatcattaatgtaagttccttggctaacgttgagaaagttgttaagccacctaaaaaaaccggtgacaaagaacgcagggaaccatgagattgaaattgataggcctatagttaatccaattaaaaaactaccaactagatttactatcaatgttgcgataggtaactttgaagtaaatttatgattaaaataatcagtaatggcacttctagcaattgcgccaaaaccgccgccaatcatgactaaaatgattgatatcatgataaaccaccacctagttttataccgacgtaacataacaaaataccaaagacataacttgttacagcatatagtagtaaagttataaattgttgatgatcaaacatatgtattaattctaattgaaatgttgaaaaagtcgttaaagcaccaagaaaaccagtcgtaatagctttttttagggtcggatggtttgaaaaaaatgcaattgttaaggctgttagcaatcccattacaaaggcaccagtcaaattggctatcagtgttccgattggaaaacctccgtcagtattcagaaaagaaatgaggtaacgtaataaagcgcctaaagcaccaccgataaaaatatatacatattgcatttggttcacctcgaaaagaagtagtttgaatttaaaaaagaggttttggcaacacgacgacaaaaattgtcgatgcattatcaaacctcattatatgttatatcttgttgtataactatagcgattagatgcatagttatgatttcgaaaatctaatattttttatacgcaacaacgtcatcaaattgttttactcattatagcatgatacattgtattgttttgtattaacgctacattgacattttatcttttttaaataaaaccgaatgtacgacaattgaaaagatatgtactaaaataacaattagaataatccaaggcaaacttttactcgcaattctaatccaatctgcatcaggctttagtgatttaattgaacgatctgcaaaaattatagacaaaattagtacaattgagttaataacactgcagaaaagtattaatttaataaaagaattaaaaaatccacttaggaaaacgttatttgtattaaagaaaaagctt

SEQ ID NO: 4 - DNA, 8654 bp, Staphylococcus epidermidis
  Designated as SE-22
  misc_feature (1564)..(1564): n = a or t or c or g
aagcttgttttattgcttagttatatttccaataacactcattttatatgtacgtattgccaaaaaaaattatctatacagtaataagtatgaaatgagaactggaataatcattggtattattgctttaattctagtaattatgcaagggtttcactttaactgggctattattcctatttctatctatggtcatcagtttgtatttttcgctggaattattttaagtcttgttggtatattctttaaacgtatagaatttgtaggagttggcttactattttgtcaaaaacatagatgcaatggtaactgacccggaaattgcacagtttttctctttagcaatttggattatacttgttgtgctaatcattttttatacgatacgtttatctgaacgcactaaatcatcatcatatacaaagatttaaactcagaaaatatgctagacatatctttctgagttttttaatttattaaaatatatcatttgtttaccatataagtttgttttagaaaatgaatcactattttaatatacaaataatttaattacactgaaaataacctaaaagcgtaacactattttaatatgggtatataaatgactaaagggaggtgccaagatgaataaaattcaaatttgtaatcagattgaacttaactatattgatgaaggcgaaggcatccccatcattttaattcatggattagatggaaacttggcaggatttaaagatttaaaaaatgaactcaagaagcagtatagagtaattacttatgatgtcagaggtcatggaaaatcttcacgaacagaatcatatgaattaaaagatcatgttgaagatttaaatgatttaatgggagcattaaatatcgattctgcacatattttaggacatgatatggggggcatcattgcgagtgaatttactgaaaaatatcaatataaagtgattacattgacaattgtttcggccaaaagtgaagacattgcaaatggtttcaacaaattaatggttgattaccaagaagaattagcaggctttaataaatctgaggcaatgattattttattctctaaattatttaaagagaaagataaagcaatgaaatgggtatcaaagccaaaaattatacaatagaccaactccggaagaaagtgcaattgcagtacgtgcattgcttaatattaaagatttaactcgtgttcatcataatgtgtccatacctactttaattgtgaatggtaagtatgacccactcatacaaaataaaagtcattatgatatggatcaatattatgatcaagttacaaaaattgtatttgataattcaggacatgcaccacatatcgaggaaccagaaaaattcctgaaactctacttagattttgttagttaaaaaataagaacataaataaaaacccttaaatgattattgtcggaaaatcatttgagggttttgtagtagcagtaaagtttggactcagatcactatcgtattaacttaataaaagagtaaaacagtcttatctttcataagtgaaagaaatatctgtttnactccctagccattatacttcatttcattatttgcttctgtgatacggttgtttactcgtttaagtaaatcatcgatttttttacgctgcttagaatctactaagattaaaacagttctttcatcgtgttcattacgttttttattaaagtaattttcttgagataaatttttaacagctttaacaacttgaggttgtttataatttaagtgattgataatatctttaagataatattcctcttctttattctcactaatataagttaatactgcaaattcttcaaagctgattgagaattcttttttaattattccttttaatctgtcagcataagtgaccatagctaataattcaaagcagtcattgatttttgaaatagccattaatgaaacctccctatttatatcatatccataaatcttaaaacccatctttttaaatttaaagatagttaattatattattgaattaagattacttggatactataccctaatttattaatttatatctatttttcttatgaaaatacgaaagtgtccgtcataatatagtattaatttaaatttaaagaatatatttaatgctatattatttagttaattataactaaataaaattaagaagtaaacaaataagtgtttataaaacaaattatcttttaaagtttatacttgaattagcaatgtagcatttgctatattcaaaaaaataagattgtttctaattttccttaatttaataaaaattatactaaaaagaatactttttggaaagaattttactaacattttttatatataaatgtttattaatttagaagtaggatttttaacaactttttcatctatcaataagcctttagttatattaatatacccactttttaaactctttttgtatgttacttctctttttgtagaattaaaacatagcgtttttgaacaatagctgacgtaggtaactctatgtcatttgaggctaatttgattttaaagtgtgttccaatttgatgattgggttgtgtagaaagtaaaatgtcgtaatatgagacgccattttttatttttgatggtatattcgaaatttctttaattttactagtaaattgagtgttgtcactagatgttacagaaatattttgatttatttttaataaattcaactcagattctgatatattagcacgaataatacgttcgttgctattaatttgcactatcttttcgtttggttttgaagggatagaattaatatatgaaatacttccattaattggtgaaaataaagtggatttaattgaggatttagtttgaatcatttgtaattttagctgattaaggaatgaataataatgtaaatcatttttagaatttaaagttttgttgttacgttcattactaagtgtattttggagttcctcatataaatgatctttttcataattgtaatattctaacactggagtgtttttagatactttgctatgattttttactaaaagtttttggagttgtcctaaagtgggagtgtagtagaaaatatagctgttaagaggggcttgtataccagttgttgaaaggagtaatttgggctttgcttttatagtttttatatttttaatatcttctgttttagaagttaatttagagaaagtaatgtaactaaaactacaagttgtgagaatgaaaatgaatagtaatgaagaaataacgatgcgttgcttggtcatggatgttcacctcataatattattgtgaggttattatacactattattttaaatgaaatatattaattttaaataagcattacttttggtttgtatattgttttatttcaaaaaataaagtaaatcaatttaataaattgaaaaatagaaggctatctttaattttaaaatatatgattctacataaatgttactataagaagaatcactcataaaaactgccaacaaagacaaaatctttgttggcagttcgaaatagacatttatttgtatgaggaatctacattaatataagcggataatttttattcagaataaggaatttaaaataatcgtaataaaataatacctatagctatacataataatccacctaacttacgtgatgttattttgtttttaggtgaacccaacaaaccgaaatgatcgataataatacccataatcatttggcccatcatagcaattatagtagttaaagctgctcctaagaaaggcattaaaataatattagatgttacgaatgccattcctagtatccctccaataaaataaatagatttaatcttacctagtgttttatgagtagatgatattttcagactacgattaaatactaatgttaatataaataacgctattgtaccaacgctaaatgatatgagtgaagcaaatatggatgagtgtgtgtgttgagccagtgtgctgttgattgttgtttggattggcggacgaaaccaaatacgaatccaataagcaaccagaatactattggtgtattcttatgtctattaacaggatgtctacgaacataattcataaatataattccagtaattaaaaatataattccaacacctttaaataatgtaaaagattgttgatgggcgcccaataatccaaatgtatcaatgattacacccataataatttgccctgtaaccgtaataacaacagtaagtgctgcgcctaatcttggtaataataataagtttccagttaaatagataacacctaatagtcctcctaggacccaagtatagttaagtgtttgcttagaaaagaattctggtgttaatacttgtggatgaataatgatattaagcacaagtaagcatattgttccgacagcaaaagatatggttgaagcataaaaagatgaacgggtaaattggcttagccttgagttgattgaagtttgaataggaagtaacatgccaacaaaaattcctaaaagatatagaaaaaacaatgataaaaaccaactttctcaatttaatatgattatcataccattcataatcatgtttctaaaatgattgagccataagcaaagtatagaaataagttgtgaatgttccgaggtgtcatacagccgatactattttgatgaatcattataataaaatgcacattaaacaagttttagaattaaaaaaagcgagacatcattttgaatttgatatctcacttcatattaataaaagaacaatgtaaattaagttcttttttagacttgaacaattttaaaaaatttgttcttcgataagtcttttttatgattttagtactttaaataaagcgtcaaaaataatgttttatgaattaatttttatcttcaaatataacagttgtccttttatcaataagttgtgcagcataaattttgacaggctttcccaaactaaatcttaaaatgtctaattctaaaatgtctaattctaaaagttggttcatactttctttaattaattgttctgtagtaatagcgttaaaatcgggtaatagtaatttgacgggtttattaagatttgatttaaatacgagttccaaagtttttgacatactgatgtatcctccttaaattaaagattctgttttaacgatctcgactttgtcatactcttcgccactgaacgttcaatgatggaacgaaaagatttgatttgatcattagaaacaagcggattaatgttagaaaaacgacgcttatgttcgactactttaccttcagaattatgtttgatttgagtaaagataatcgtcacttgattgacttcattcataataaaacctcctttcactatatatatcgaaatagattgaaaaaaaaggacacattttttgaaaaatataggcaaatgcctttgatgtgatacaaacgtcatttatcattaattatgaaacctgttttagaaggtatatgaggtaagtagaattgttaagttgtaaaagaaaaaattggaacctgatatttaaaataaccaacttaaaagattgatcagtgtctaaaattactatttatatatgaattaaaatattaagatctcccaatatgagaatgaattagtttaagtttatcgatgattgaaaaattatagcctcatggattctatcttatataaaataaagttctattcccttttggatataaataagaatagttacctttttgtgatatgccaattcagaaaaaaagcgacagtgcttgaatctatgtatgctcaataaactcattcaaatcaactagcaatatcaaatcataaatcgtgttgcaccataataaggattaaaacctgttagtttaactaatttaagaaaaacatttgattatcttctctttcaatcgggaatattaatttctatcattcaacaatattttggatatcagataacttaagaaatattgagatttattgaaatacgatatgtttcaaatcgccatacaatgattacacttaataaatgattacacttaatataaatgtaaaaagaaaaggaggggttaaatgagtttagtatatcttatggcgactaatttattagtcatgctcatagttttattcactctgagtcatcgtcaactaagaaaggttgcgggctatgttgcattaatagctcctattgtgacatctacatattttattatgaaaataccagatgtgattcgaaataagtttattgctgttcgattaccatggatgccttcaattgatattaatttagatttaagattagatggtttaagtttaatgttcggcttaattatttcgctaataggtgtgggtgtatttttttatgctacgcaatatttatcccacagtacggacaatcttcctagatttttcatctatttactattatttatgttcagtatgattggcattgtaatagctaataataccatcttaatgtatgtattttgggaactcacaagtatttcctcattcttgcttatatcctattggtacaataatggtgaaagtcaattaggcgccattcaatctttcatgattacagtgtttggtgggctagcgttattaacaggatttatcattttatatatcattacaggaacaaacacaattactgatatcttaatcaacgcaatgcaatttcacgacatcctttatttataccaatgattttgatgctattattaggtgcttttaccaaatctgcacaatttccgtttcatatttggttaccaaaggccatggcagcacctacaccagtaagtgcttatcttcattcggcaacaatggtaaaggctggaatctttttactatttagatttacacctttattgggacttagtaatgtttatatttatacagtgacatttgttggtctaataactatgttatttggatctttaactgctttacgacaatacgacttaaaaggtatactcgcttattctacaataagtcaattaggtatgattatgacaatggtaggtctaggtggcggttatgctcagcacacatcagatgaattgtctaagttttatattttagttttatttgctggcttattccatttaatgaatcatgcggtttttaaatgtgcattatttatgggcgttggtatcattgatcacgagtccggaacacgtgatattcgtttgctaaatggtatgcgtaaagtctcccctaaaatgcatattgtcatgttgctcgctgcattatctatggcaggtgttccttttttaaatggctttttaagtaaggaaatgtttttagattcgttaactaaagcaaacgaacttgatcaatatggcttcgtattaacgtttgtgattatttcaataggtgtcatcgcgagtatattgacttttacttatgcactttacatgataaaagaaacattctggggaaattacaatatagaaaaatttaaacgtaaacaaatacatgaaccatggctatttagtttaccagctgtgattttaatgttactcattccagttatcttctttgttccaaacgtttttggcaactttgttattttgcccgcaaccagatctgtatctgggatagggcggaggttgatgcatttgtgccacatatttctcagtggcatggtgtgaatctccattaattttaagatagtgtatatattggactattttagctctagtgtgattggaaagaggttacgcatcaaataatcaaaagtgctcgattacagtggctatcggaaatttatagagaatttgaattatactcagcccgtggtatacgtgcattgatgaataataaattgaattattacatcatgattacattatttatttttgtagctattgtagttatggatatttgactgtgggttttcctcatgtactcagcttcatattagttctttcggaccgttggaagttatcttatcagttgtaacattgattatcggcatttcattaatctttattcgtcaacgactaacgatggtggtattgaatggaatgattggattcgcagttacattatattttattgcaatgaaagctccagatttagctttaacacagttagttgttgaaactattacgacaatcttatttattgttagtttttcgagactacctaacatccctcgagttaaggcaaatttaaaaaaagagaccttcaaaatcattgtgtcacttgttatggcattgacggtggtatcacttatttttgttgctcaacaagcagatggtatgccttcaattgctaaattttatgaagatgcatatgaacttacaggtggaaaaaatattgtcaatgctatactaggtgacttcagagctttagatactatgtttgaaggactagtgttaatcatagctggattaggtatttatacgttacttaattacaaagataggagggggcaagatgaaagagaatgatgtagtacttaaatcagttacaaaaattgtagtgtttattttgttaacatttggattttatgtattttttgctggccataataatccaggtggtggctttattggtggcttgatttttagctcggcatttatcttaatgtttcttgcctttgatgtaaatgaagtgttgaaaaaagctt

SEQ ID NO: 5 - DNA, 2362 bp, Staphylococcus epidermidis
  Designated as SE-3
aagcttcacaacttgaaaatatagcacaaacattaaaggatttaggtagaaaacgagcaattttaattcatggtgcaaatgggatggatgaggccacgctttctggtgaaaatatcatttatgaagttagcagcgaaagagcattaaaaaaatatagtttaaaagcagaagaagtcggtttagcttatgcaaataatgacacgttgataggtggttcacctcaaacaaataaacaaattgcattgaatatcctaagtggcacggatcactcaagtaaacgagatgtagttttgttaaatgctggaattgctttatatgttgctgagcaagtggaaagtatcaaacatggcgtagagagagcgaaatatctcattgatacaggtatggcaatgaaacaatatttaaaaatgggaggttaagtaatgactattttaaatgaaattattgagtataaaaaaactttgcttgagcgtaaatactatgataaaaaacttgaaattttacaagataacggaaatgttaagaggagaaagctgattgattcactttaactatgatagaacattatcagttattgctgaaataaaatcgaaaagcccatctgtacctcaattaccgcaacgtgatcttgttcaacaagttaaagattatcaaaaatatggtgctaatgctatttcaatattaactgatgaaaaatactttggcggtagttttgaacgattaaatcagttatcaaagataacatcgttaccagttttatgtaaagattttattattgataaaattcaaatagatgttgcaaaacgagctggtgcatctattattttattaatagtaaatattttaagtgatgaccaattaaaagaattgtattcatatgcaacaaaccataatttagaagctctagtagaagttcatacaattagagaacttgaacgtgcacaccaaattaaccctaaaattattggtgttaataatcgtgatttaaaacgatttgaaaccgatgttctacatacaaataaattacttaagtttaaaaagtctaattgctgctacatttcagagagtggcattcatacaaaagaagatgttgagaaaatagtagattcaagtattgacggtttacttgtaggggaggcattaatgaaaacaaatgacttaagtcagttttttgcctagtttaaagttaaagaagaatctctatgatagttaaattttgtggttttaaaaccgaaagtgatattaagaaaattaaaaaattagaagttgatgcagtagggtttatacattatcccgatagtaagagacatgtctcactgaaacaattaaaatatttggctaaaatagtgccagatcatatagagaaagtagtgtcgtagtaaatcctcaaatgtccaccataaagagaataattaatcaaactgatattaacacaatccaattacatggaaatgaaagcattcaattaattagaaatattaagaaacttaattcaaaaataagaatcataaaagcaattccagcaacaagaaatttaaataataacattcaaaagtataaagatgagatagactatgtttattatagatacaccatcaatcacatacggagggacaggtcaaagttttgactggaaattattaaaaaaaataaaggcgttgattttctcattgcggtggtttggattttgaaaagataaaacgattagaaatatattcatttggacaatgtggttatgacatctcaactggcattgagtcacataatgaaaaagattttaataagatgactcgaatattaaaatttttgaaaggagacgaatgattaatgaaaattcaaacagaagtagatgaattgggctttttcggtgaatatggtggccaatatgtacctgaaacattgatgccagctattattgaacttaaaaaagcatatgaggacgcgaaatcagatactcacttcaagaaagaatttaattattatttaagtgaatatgttggtagagaaacgcctttaacatttgctgaatcatacacaaaattgttaggtggtgccaaaatatatcttaaaagagaagacttaaatcacactggtgctcataaaattaataacgcgataggacaggcactattagctaaaaggatggggaaaactaaattagtagccgaaacaggtgctggtcaacatggtgtagcaagtgccaccatcgctgctttattcgatatggatcttattgttttcatgggaagtgaagatatcaaacgtcaacaacttaacgtatttagaatggaattgctaggagctaaagtagtgtctgtgtcagatgggcaaggaacactatcagatgctgtaaataaagctt

SEQ ID NO: 6 - DNA, 5024 bp, Staphylococcus epidermidis
  Designated as SE-32
aagctttttgatttttaaagaaaaaattaaacaagggggcattgcttatggtcaatagaagaaagatatcaattattggcgcgggacatacaggtgggactctagcattcattcttgcacaaaaggaattaggagatattgtgttgattgaacgccagcaatcagagggtatggctaaaggaaaggcgttagatattttagaaagcggacccatttgggggtttgacacatctgtacatggttcagtaaatatagaagatattaaagattcagacatagtggtgatgactgcaggtatacctaggaaatcaggaatgacaaggagaagaattagttcaaactaatgaacaaatagtacgagaaactgcattacaaattgcaacgtatgcacctcattcaataattattgtattgactaatccggttgatgttatgacatatactgcatttaaagcatcaggttttcctaaagaacgtattattggtcaatctggaattttagacgctgcaagatatcgaacttttattgctcaagaacttaacgtgtctgtcaaagatgtaaatgggtttgttttaggtggacatggtgatacgatgttacctttgattaataacacacacattaatgggattccagttaagcatcttatttctgaagaaaagattgatcaaattgttgaacgtacacgtaagggtggtgcagaaattgttgcattactaggtcaaggctcagcatattatgcaccagcaactgctatatatgaaactatagatgcaatttttaatgatcggaaacggttattaccaagtattgcttatctagagggagaatacggttgttcagatatttgtttcggagttcctactataataggatatcaaggaatagaaaagattatagaggtagatatgaataatgatgagtatcaacaactacaacactctgcgcaagatgtgagtgaagtcaaaaactcactaaaattcaaataaataattatgaagttctacatcttaaattgttagatttttgtgaaaattgtgtaaagggtattttttcgttgatttataaaagcgctttcttgatataatgaacatatattcatagaataaggagacgattaaaatggctaaaggggaccaatatcaagctcatactgaaaaatatcatgagtaaaaagtctaaaaaaagttataaacctgtgtggattatcattagttttattattttaattacaatcttgttattacccacaccagcaggattacctgtaatggctaaagcagcactagctattttagctttcgctgtagttatgtgggttacagaagcagttacttatccagtttctgcaacattaattttaggattaatgatacttttactaggtttaagtccagttcaagatttatccgaaaaacttggaaacctaaaagtggcgacataatactaaaaggtagcgatattttaggaacgaataacgcgcttagtcacgcttttagtggtttttcaacctcagccgtagcacttgtagctgcagcattatttttagcagtagctatgcaggaaaccaatttacataaacgacttgcattatttgtgctatcaattgttggaaataaaactagaaatatagtcattggtgctattttagtatctattgttctagcattctttgtaccatcagctacagcacgtgctggtgcagttgtcccaatattactgggaatgattgctgcatttaatgtgagtaaggatagtagacttgcttcattattaattattactgctgtacaagcagtttcgatatggaatataggtattaaaaacggctgcagcacaaaatattgtagccatcaattttattaaccaaaatttaggacatgatgtatcatggggagagtggtttttatatctgcgccgtggtcaatcattatgtctatagctctttattttataatgattaagtttatgccacctgaacatgatgcaattgaaggtggaaaagagttaattaaaaaggaacttaataaattaggaccagtcagtcatagagaatggcgactaattgtgatttcagtgcttttatattctctggtcgactgagaaagtattgcatccgattgattcagcttcgattacactagttgctctaggtattatgctaatgccaaagattggtgttattacttggaaaggtgttgaaaagaagattccttgggggacgattatagtatttggtgtaggaatctcacttggtaatgtattacttaaaacaggagccgctcatggttagtgatcaacatttgtttgatgggtcttaaacatttaccgatcatagcaactattgcgttaattaccttatttaatatattaatacatttaggttttgcaagtgcaacgagcttagcctctgcgttaatacctgtgtttatttctttgacttcaacgctaaatttaggtgatcatgctattggttttgtattaatacaacaatttgtgattagttttggtttcctactacctgtcagtgcaccacaaaatatgcttgcatatggtactgggacttttaccgtaaaggattttttaaagacaggtatacctttaacgatagtaggttatattttagttatcgtatttagtttaacgtattggaaatggcttggtttagtgtaagtaaaagatttaggtattaaaatgataattataaatgtctcgtaaagtttaatattttaactttacgacacattttttataaactcgtggcaagttaatcttaatagttgaaatgtatcgtataaaaaatatatgaatgtaaatagaatttagtattagagaataacaaaaaattgatgttaggtggtaaaatctaatggctataggtgtcatattaaatagagtttttaggctaaataataatccattatttgattatatatatagtaataaagaatctataaatcattgttattttattattccaactgaagagtttgaagaagaagcaaaaaagaaagcacaatactattatgggtccatacagaagtttatgtatgaactacaacgatatgatatagaaccctttttgatgtcttatgataaattaatagacttttgtaaaaaacaagctatagacaaagttgttgttgcaggtgatattatgagttatcatcacgaagaatatgacattttacatcaaaggaaacgatttaaacaagctaatattcaagtaatatcattaagagcaaatcattattttaacccccgcaaaacacataataaacaaggggaaccatataaagtatttaccagtttttatagaaaatggcgtccttacttaatgattagagatgaatatgactatcatttagaagatatttcaaaggttgtagtgaaatctcaacataaaattaaagaagattatcattcatatggtataagtgaacgtgatgttcaaaatcgttggtctgaatttttatctcaagatatcgaaaattataaagaaaacagggaatacttgcctgaagtattaacaagccaactaagtatttacttagcttatggaatgatagatattatacaatgttttcaacgatttacttcaaaattatgataaaaatgaacaaaattacgaaacttttatacgtgaattgatttttagagagttttattatgtattaatgaccaattatcccgaaacagctcatgttgcttttaaagaaaaataccaacaattgaaatggtcttataatgaagagaattttaaactgtggaaagatgggaatactggttttccaattattgatgcagcaatggaggaacttaaaacaactggatttatgcataatcgcatgagaatggtagtttctcaatttttaactaaagatttgtttattgactggatttggggtgagtcatttttcaaacaaaaattaatagattatgatgcagcttcaaatgttcacggatggcagtggtcagcttctactggaacagatgctgtaccatactttagaatgtttaatcctataagacaaagcgagcgttttgataataatgcacgatatataaaaacttacattccaagattaaatcaggtagatgctaagtatttacacgatactcataaattcgagcaacaaataaaggggcaaggtgttgaaataggtaaagactatcctaaacaaatgattgatcacaaagaaagtagacaacgtgtaatgtcagaattcaaagctatagattaaataaaaaagatctgaacaacatgatataggtgttcagatctttatctagttacataaaaaagcaaacatgaattaaaatatattctaacaaagttaaaatatacatatatttaagatttaatttagttttcaaaggtacttcccaatttgtataacggggctcataataaaataattgcatcaaatataatcctatccctaacggtaaacacattaataaaatagctttagtataactccatcctatttgatgccataaatgacctatcataagttgaataatgatgagacataccattaaaattacttcaattatcattggtataatctcacccctttaataaacaatatgactgttgcttgtatgagcaccattaaaacgacaaatagtaacgctttaacatctatgattaaaaaaacctctttcacaatttttaaaggtgcatttaataaatagacagtatgtaatcttaagaatcgaccgatgtaaatacctaatccatttaagaacattaatataactatcaatagtcgatttaaccatacataagacgtaaaatgtgcaatttctaaaaatataagaattgtgaggtatattgctaagagtacgccaagtattaaataggtgaaataaatccattctgtgatgtttaatccagctaaaaagttaaattgaaattggtttaagtgtatgagatcggtaatcatataaaatgtgtttggaactaataatagaaatatgagtccgaaaacaataaataagggccattcaaaagctt

SEQ ID NO: 7 - DNA, 9515 bp, Pseudomonas aeruginosa
  Designated as P2-2
  misc_feature (8841)..(8841): n = a or t or c or g
  misc_feature (9111)..(9111): n = a or t or c or g
aagctttcctccagacccttcaccgccgtggagatcgacggctgggcgatgtacagcttgcgcgaggcctcggccacgctgccgcattccacggtggtcacgaaatacttgagttgccgcaaggtataggacgccactgcaagacctcatcggcgcatcatcctccccgggccgggcgtgcgcgcctcgattgttgtgtccgccgcgctgcaagcaagttgcaggccgctgccgagcgtcgcgcgctggccgcggaacgattgcccgcctgcacgataacccagcacgacgcactttgccggggcacgcctggccagctttttcttatgtcccgaggacatttttaataattttccttcgccgcggcttgcgcgaccatccttccccatcgaccccatggacagcggttcgcctcccggcggtccgggccatgcgtgcagaaccacgaccggcgcagaccggcgagataacaaggagaaggtggggtgttcgaactcagcgattggcaacggcgcgccgcgacacagcgcttcatcgaccaggccctgatcggcggccgccagcgtccagccgccagcggcgctaccttcgacgccatcgatccggcgagcaatcgcctgctggcgcgggtcgcggcctgcgatgcggccgacgtcgacgcggcagtggccgccgcccgccgcgccttcgacgaaggcccctgggcgcgtctcgccccggtcgagcgcaagcgcgtgctctgcgcctggccgagctgatgctggcccatcgcgaagagctggcgctgctcgactcgctgaacatgggcaagccggtgatggacgcctggaacatcgatgtacccggcgccgcccacgtcttcgcctggtatgcggaaagcctcgacaagctctacgaccaggtcgcgccggccgcccagcagaccctggccaccattacccgcgtgccgctgggggtgatcggcgcggtggtgccgtggaacttcccgctcgacatggccgcctggaagctcgccccggccctggccgccggcaactcggtggtgctcaagccggccgagcagtcgccgttctccgccctgcgcctggccgagctggccctggaggcgggggtgccggaaggcgtgctgaacgtggtgccgggcctcggcgagcaggccggcaaggccctcggcttgcacccggaggtggacgcactggtgttcaccggctccaccgaggtcggcaagtacttcatgcagtattccgcgcaatccaacctcaagcaggtctggctggagtgcggcggtaagagtccgaacctggtgttcgccgattgccgcgatcttgacctggcggcggaaaaaggcgccttcggcattttcttcaatcagggcgaggtctgttcggcgaactcgcgcttgctggtggagcgttcgatccacgacgagttcgtcgagcgcctgctggccaaggcccgcgactggcagccgggcgatccgctggacccgggccagccgcgccggcgccatcgtcgaccgccggcagaccgccgggattctcgccgccatcgagcgggcgcaaggcgagggcgcgaccctgctcgcggtggccgccagttgacgatcaacggttcggacaacttcatcgaaccgaccctgttcggcgacgtacgcccggacatgcagctggcccgcgaggaaatcttcggcccggtgctggcgatcagcgccttcgactccgaggacgaggccatacgcctggccaaggacagccgctacggcctcgccgcctcgctgtggagcgacgacctgcaccgtgcgcaccgggtggcgcggcgcttgaatgccggaacgtgtcggtgaataccgtggacgcgctggacgtcgcggtgcctttcggcggcggcaagcagtccggcttcggtcgcgacctgtcgctgcattccttcgacaagtacacccagttgaagacgacctggttccagttgcgctgaagacgcgacggacgcgacacgactcgatgccgataacgacaacaagaggacgatcgaatgaacgacacgccgaacgtgcgtgagccggccctgcgccgcgtgctcgggctgggaccgctgctggcggtggccatcggcctggtggtttcccagggcgtgatggtactgatgctgcaaggcgccgggacggccggcctgggcttcatcgtgccgctgggagtggcctacctgctggcgctgactacgccttttccttttccgagctggccctgatgattccccgcgccggtagcctgagcagctacaccgaggtggccatcgggcatttcccggcgatcctggcgaccttttccggctacgtggtggtggcgatgttcgccctctcggcggaactgctgctgctcgacctgatcatcggcaaggtctaccccggcgcgctgccgccgatgctggtgctacggcgtgctcggcctgttcaccctgctcaacctgctcggcatcgacatcttcgcgcgcctgcagagcgcgctggcgctgctgatgatgatcgtcctgctggtgctcggcctgggtgcggtgagcagcgaccacgcttccgcgcagaccgccctggcgagcggctggaacccgctgggggtaagcgccctggcgctcaccgcgatggccgtgtggggcttcgtcggcgccgagttcgtctgcccgctggtggaggagacgcggcgtccggagcgcaacatcccgcgttcgatgatcctcggcctgagcatcatcttcctgaccatcgccctctactgcttcggtgcgctgctgtgcatcccgcaggcggaactggccggcgacccgctgccacacttcctcttcgccaaccgcgtgttcggcgagtacggccagctgttcctggtgatcgccgcgatcaccgccacctgcagcaccctcaactcgtcgctggcggcgatcccgcggatgctctacgggatggcgcagaacggccaggccttcccgcaattcaagcagctcagccggcgggcgcgcacgccctgggtggcggtgctgttcgtcgccgcgatcaccggcctgccgatcctgatcctcggccaggacccggactcgatcaacctgctgctgctcgccgccgcgctggcctggctgctggcctacatcatcgcccacgtcgacgtgctggccctgcgccgtcgctatccgcacatcgcccgtccgtttcgcacgccgttctacccgctgccgcaactgttcggcatcgccgggatgatctacgcggtggtccacgtctcgccgaccccggaaatgaccggacggatcttcgccagcgccggcgtggtgctcggcgtggtctcgctggtggcggtggtgtggatcaagggcgtgatgcgcaagcccctcttcgtacccgaaccgctcgagacggccggtgagactgcccagggcaagtccgtcgccctcgatcccctgcaatcccttcggcctgacgcgccaagggaacaaggagaacacagacgatgaccgctcagctcaacccgcagcgcgacacccgcgactaccagcaactggacgccgcgcaccacatccacgccttcctcgaccagaaggcgctgaaccgcgaaaggcccgcgggtgatggtccgcggcgatggcctgcagctctgggacaacgacggcaagcgctacctggacggcatgtccggcctctggtgtaccaacctcggctacggccgccaggacctcgccgccgccgccagccgccagctggaacaactgccgtactacaacatgttcttccacaccacccacccggcggtggtggagctttccgagatgctcttcagcctgctgccggaccactacagccacgcgatctacaccaactccggctccgaggccaacgaggtgctgatccgtaccgtgcggcgctactggcagatcctcggcaagccgcagaagaagatcatgatcggccgctggaacggctaccacggctcgaccctgggcagcaccgcgctcggcgggatgaagttcatgcacgagatgggcgcatgctgccggacttcgcccacatcgacgaaccctactggtacgccaacggcggcgagctgagcccggccgaagttcggtcgccgcgcggcgctgcaactggaggagaagatcctcgaactgggcgcggagaacgtcgccgccttcgtcgccgagcccttccagggcgccggtggcatgatcttcccgccgcaaagctattggccggagatccagcgcatctgccggcagtacgacgtgctgctgtgcgccgacgaagtgatcggcggcttcggccgcaccggcgaatggttcgcccacgaacactttcgcttccagccggacaccttgtccatcgccaagggcctgacgtccggctacatccccatgggcggcctggtactcggcaagcgcatcgccgaggtgctggtggagcagggcggggtgttcgcccacggcctgacctattccggccacccggtggcggcggcggtggccatcgccaacctcaaggctgcgcgacgagggcgtggtcacgcgggtcagggaggagaccggcccctacctgcaacgctgcctgcgcgaggtcttcggcgaccatccgctggtcggcgaggtccagggcgccggcttcgtcgccgcgctgcagttcgccgaggacaaggtgacccgcaagcgcttcgccaacgagaacgatctggcctggcgctgccgcaccatcggcggcttcgaggagggcgtgatcatccgctccaccctcggccgcatgatcatggccccggcgctggtggccgggcgtgccgagatcgacgaactgatcgacaagacccgtatcgcggtggatcgcaccgcgcgcgagatcggcgtgctctgacgcgccccggcggcccggcctcggccgggtcgcctgcgacacggagcgtccccccataacgacgatgcggcgcctggcgaccgcgcgcggaaccgtttcggcctctggcggcaactgcctaagcaacatcacaacaatgccaatcggctgtgggagtgttccatgttcaagtccttgcaccagtacgcacacgtgttttcccggttgtccctgttcgtcctggcgttcgccgcggcggcccaggcgcagagccagagcctgacggtgatctccttcggcggcgcgaccaaggccgcccaggaacaggcctatttcaaacccttcgagcgaagcggcggcgggcaggtggtcgccggcgaatacaacggcgaaatggccaaggtgaaggccatggtcgacgtcggcaaggtcagctgggacgtggtcgaggtggagagccccgaactgctccgcggctgcgacgaggggctgttcgaacgcctcgacccggcgcgtttcggcgaccccgcgcagttcgtccccggcactttcagcgagtgcggggtggccacctacgtctggtcgatggtgatggcctacgactcgacgaagctggccagggcgccgcagtcctgggcggatttctggaacgtccgcgagttcccccggcaagcgtggcctgcgcaagggcgccaagtacaccctggaagtggcgttgctggccgacggggtgaaggcggaggacctctacaaggtactcgccaccccggagggggtcagccgcgcctttcgccaagctcgaccagctcaagccgaacatccagtggtgggaggccggcgcccagccgccgcaatggctggcggccggcgacgtggtgatgagcgcggcctacaacgggcgcatcgccgctgcgcagaaggagggggtgaaactggccatcgtctggcccggcagtctctacgatccggagtactgggcggtggtgaagggcaccccgaacaaggcgctggcggagaaattcatcgccttcgccagccagccgcagacgcagaaggtgttctccgagcagatcccctacgggccggtacacaagggcaccctggcgttgctgccgaagacggtgcaggaggcgctgccgacccgcgccggccaacctcgaaggcgcgcgggcggtggatgccgagttctgggtggaccacggcgaggagctggaacagcgtttcaatgcctgggcgcgcgctgagcgctgcgcgtcggcaaaaaaaatgacgggccccaagtcgtccgggcccgtcgggtcaaagcgctgacggggtgatcagcgcagctcttccaacaacccctgcagataccgacagccctcggtatccagcgcctgcaccggaaggcgcggcgcccccacctccaggccggagaggcccaggccggccttgatggtggtcggcaggccccggcggaggatgaagtcgagcagcggcaactgccggtagaacagcgcgcgggccttctccaggtcgccgtcgagcaccgcctggtagagctggccgttgagcgtcgggatcaggttcggcgcggcgctgcaccagcctttcgcgccggccacgaaggcctccagcgccagcgcgttgcagccgttgtagaagggcacccggccttcgccgagcaggcgcagcttgtgcatgcgctggatgtcgccggtgctctccttgaccatggtcacgttgtccacttcgcggacgatgcgcaggatcagttccaccgacatgtcgatgccgctggtgcccgggttgttgtagagcatcaccggcacgccgatggcttcgccaaccgcgcggtagtgctggaacacttccgcctcgttgagcttccagtaggagatcggcaggaccatcaccgcctcggcgccgagggattcggcgaactgcgcgcggcgcacggtcttggcggtggtcaggtcggagacgctgacgatggtcggcacgcgatgggcgacggtcttcagggtgaagtcgaccacctcgtcccattccgggtcgctcaggtaggcgccttcgccggtgctgccgagcggggcgatggcgtgcacgccgccgtcgatcaggcgctcgatggagcggccgagggccggcaggtcgagaccgccgtcggcgccgaaggggggtgatggtgtagccgatgatgccgtggatggatgcggacattggatgtacccgtgacattgagtgggaaatgccaggacggacctggtgggaaaggtcgttcagctcaggcagtcgctgttgcgcggcaggcagcgccgggcgtagtagttgaatgcggcgccgtggcgcttcggggtggagatccagtcgtgggcctcgcgcgccagggccggcgggatcggcttgatctctccggcggccatcgccagcaactgcatcttcgccgcgcgctcgagcagcaccgcgatcacgcaggcctcctcgatgctcgcaccggtggccagcaggccgtggtgggagagcaggatggcgcgcttgtcgccgagggcggcggagatgatctcgccttcctcgttgcctaccggcacgcccggccagtccttgaggaaggcgcagtcgtcgtatagcgggcaaaggtccatgtgcgagacctgcagcggtacttccagggtcgacagcgcggcgatgtgcagcgggtgggtgtggatgatgcagttgacgtccgggcgggcgcgatagacccagctgtggaagcgattggccggattcgccatgccgtgcccgtggaggacgttgaggtcttcgtcgaccagcagcaggttgccggcgctgatctcgtcgaagcccaggcccagttgctgggtgtagtaggtccccgcctccgggccgcgcgaggtgatctgcccggcgagcccggagtcgtggccggcctcgaagagaatccggcaggtcagggccagcttttgccggtcagtccacgtattatcgccgaggctgcttttcatctgcttcagcgcgtgctggatcagttgatccttgggtaattccagtgtcgtaaccatgcgaggttcctttgacggagcgagtcgggggaaacgccaggcagttgcgcgccacgcaacgacccggctgtaaatgacacggatcaagttatatgacacaaagtgtcatttagcaagagagaagtttcatcgccatcgggagaaggctgtcctcaatgtccatgcgcttgaaattgctgagaaaaaaactcggggtcacgctggagaccctggccgacaagaccggcctgaccaagagctacctgtccaaggtcgagcgcgggctgaacacgccgtccattgccgccgcgctgaagctggcgaaggcgttgaacgtgcaggtggaggagctgttctccgaggaaagcgacggtgtcgacggctacagcatcgttcgtcgcgaccagcgcaagtcgctgtccagcggcgacgacggcccggcctacgcctccctcgtcgcagcagatcggcgcccgcgcgctgttgccgttcatcgtccaccccccgcgcgatttcagtcactcgacgttcaaggagcacctcggcgaagagttcatcttcgtccatgagggccaggtcgaggtcgacttcatgaaccagcggatcatcctcgagcgcggcgacgccctgcatttcaacgcacagaagccgcaccgcatccgctccctgggggagacccaggcggaattgctggtggtgatccacagcgacgaatgaggcgacggcttcggtcgatcggatgcttgctaacgttctgttcgattatcgaactgttaatcgattatcggattgtgagccctcggaccccggcgtaaggttctcgtcacgtgccgtccaggcagcgcacaacaagacgagacccgaccgatggctgaaatcctctccctgcgcgaacggtgcgacgcttcgtccacgatggcgacagcgtcgccctcgaaggcttcactcacctgatcccgacgnccgccggccacgagctgatccgccagggcaggaaagacctgacgctgatccgcatgactcccgacctggtctacgacctgctgatcggtgcaggctgcgcgaagaagctggtgttctcctggggcggcaaccccggtgtcggttcgctgcaccgcctgcgcgacgcggtggagaagggctcggccgcaaccgctggagatcgaggaacacagccacgccgacctcgccaacgcctattttgccggcgcctccgggctgcccttcgcggtntgcgcgcctacgccggctccgacctgccgaaggtcaacccgctgatccgcagcgtcacctgcccgttcaccggcgaagtgctggcggcggtgccctcggtgcgtccggacgtcagcgtgatccacgcgcagaaggccgaccgcaagggcaacgtgctgctctggggcatcctcggcgtgcagaaggaagcggccctggcggcgaagcgctgcatcgtcaccgtcgaggagatcgtcgacgaactggacgccccgatgaacgcctgcgtcctgccgagctggggcgctcagcgccgtgtgcctggtgcccggcggcgcgcatccgtcctatgcccacggctactacgagcgcgacaaccgcttctaccaggactgggacccgatcgcccgcgaccgcgaaagctt

SEQ ID NO: 8 - DNA, 2291 bp, Enterococcus faecalis
  Designated as EF-1
  misc_feature (2096)..(2096): n = a or t or c or g
aagctttagataatgataaacgcgtgtatgtgaatgtccagccgattcaatcgcctactggagaaacagtgattggtgtcctttatgtgaaaagtaatttagaaaataaataccaagaaattactaacacagcaagtatctttttcactgcttctattattgccgcagcaatctcgattattgtgaccctactgattgcacgatcaatcacgaagccgattggtgaaatgcgcgagcaagccattcgaatcgctcgtggtgattacgctggaaaagtagaagtccatggaaaagatgaattaggccaattagcagaaacatttaatcaattatcagaacggattgaagaagcacaagaaacaatggaagcagaagaatcgtttagatagtgtcttaacgcatatgacagatggtgtcattgcgacggatcgccgcggaaaggtgattacgattaatgagatggccctttcattattaaatgtaaaaaatgaaaatgtgattgggacctcgttattagagttgttagatattgaagaagattacacattgcggaagctgttagaagagccagatgaactgctgattgatcgctcaacgtctgatcgtgaagaagaccaaatgattatccgggtagactttacgatgattcgtcgggaatcaggatttattactggcttagtttgcgtacttcatgacgtcacagaacaggaaaaaaacgaacgggaaagacgggaatttgtttccaatgtttctcatgagttgcgacgcctttgacaagtatgcgtagttatatagaggctttgagtgaaggagcttgggaaaaccctgagattgcgccgaatttcttaaaagtcacgttagaagaaaccgaccggatgattcgtatgattaatgatttgttaaatttatctcggatggactctgggaatacacatcttcaattagagtatgtgaattttaacgaattgattaattttgtcttggatcgctttgatatgatgattgaaaatgagcaaaaaaattacaaaattcgccgtgaatttactaaacgcgatttatgggtagagttagatacagacaaagtaattcaggtttttgacaacattttgaacaatgcgattaagtattcgccagatggcggcgtcattacctgccgactagttgaaacacataataatgtcgtctttagtatctcggaccaaggtttgggcatccctaaaaaagatctcgggaaagtcttcgagcgtttttatcgtgtggataaagcacgtgcgcgagcacaaggtgggactggtttaggtttagcaatttctaaagaagtaattcgggcccataacgggagtatttgggtggaaagtacagaaggtgaaggatcaactttctatatttcactaccatatgaaccttatgaagaggattggtgggaatgatgaaaaaatcagaatggattacaagaattggcttgattttgatggtcattttaagtatatatttttcagtcaatatctggctgaattctgccaaaaaaataccagaaatgaagtcgggaagccaagtcacaacagctgtcaatgaaaaagccattggcgatgtctatttacctttgcaattgattcgaatagccgatggaaaagcgatgcaaagtaatcgtgaaacattaattagtaatgttcaaaatgatattaaaatggctacgtttggtaaattgacacaagttgtgacaaaaaatgcagagcaacttaagcgctacaaccaaatggaacaaggcattgaacttctttatcaaggtccctttttaatctcggactatgcttcgatttataatctatccattaattttactaactttaatgagttgacggaccagtattttacgaaaattcaattggattttaacgaaaataagatacgttttttagattatgatcaatccaacgtctatgaagcgcccatgactgttaataaggcgcgcttaatgggaattatcaataaagagggattgcaatatcaagacgtttccgaaaatacgctaaccaaacaaggacaatgttatttaaccaatgatatgaagttgaaaaagtacagttatatcttanttcgcaaccagttactcgttttaggaatgcttttttcaatgaaacggaagatatccaaaccaatgaagacagtcaagacttaacctatacgagtaaagaagaacgattgtttgcagaagaaaaactggggaaaatcgattttaaagggaccttgccagaagagaataaacgggactcaatctataatcaaagctt

SEQ ID NO: 9 - DNA, 2441 bp, Enterococcus faecalis
  Designated as EF-27
  misc_feature (1040)..(1040): n = a or t or c or g
aagcttctgcgctaggaaccagccctttaattacatctccccatactggatttgacaatgccacttgataagcaaaaatcacaaaaataacaacaattaaagcaacaacaatagcttcaatttttctaaaaccaatttttgtcaataacaacaaaagtaaaacatcaaataccgtaatgaagacagccagacctaaaggaatatgaaataataaatataaggcaattgcgcccccgataacttcagcgatatctgtagccataattgctaactctgttaaaatccataatacaatacctaacgtcttactagttctagcacgaatcgcttgtgctaaatccatctgtgaacaatgcctaatttagcagccatatattggagcaacattgcaatcaaactggaaattaaaataatcgacatcaataaatattgaaaattttgtcccccagtaattgaagtagaccagtttcctggatccatataccccactgctaccaatgctcctggacctgagtaagcaaataacgttttccaaaaactcatatttttaggcacgtcgatggtgccattaatttcttcaagcgaaggaccatttgcatattcaatcaaatgatgtctttgctttggttcatgttcttctgaatttttcaattcaattccttctttcgttttgcaataattttaaaaggcccttcccgttagaaggttaacctctagtatattttaggtacacctaaaatatactgctaaaaataacaaaatgcaagacttgaaagaaaattttgacagtgtaaaaatagattgtcgtaaatgtgcgatcttaaagtttgaagaaatcagggtagctggtagttgattatcttaagaagtagaaaataagggacctaagtcatttcggcttaggtcccttattttatttttattcggttattctattaagaatggatgctacaatttctgtcgtgtcagctgaatgatttctaaaatctcgtaaacttaatctgacgaaaaccttcaagtacttcgggcaacttattttncccccattcaaaagttccatcatttcttttcaataatctttgtaaaatttcttctttctcgaccgctaacaaaaaatgataaacgtcaatgcctgctcgtctcagatatccaatcagctcttcttcatattcatttttataaagggtcattgtaacaataatcggccgtccagactctttggacattcgttttaataaatgagcattccagcaacgccattcctgatactcctgaaaatcattttctttcatttcttcgggaactagctccatcaatgcactaccaataatttctggatcataaatgattgcgttgggaagtttttgttgtaactcatgtgcaatggtcgtttttccggatccaaacgcaccgtttaaccaaataattatcataatttccttttcttctgaacaaatttctttgttgtttaatttaggtgctagattacttttaatttttttagccattcacttatagttactacttacatctttaacagtaaacgagacaaactaaaaatacaacatcctacgctattaacctcgggttatataacatactcatctgataatttctccctaaaaaaacagaatgtgggcaatctttttaagaataattgaatagaataacaacaaacagtaattcaggtataaccagctagaaattgttttatttttagtcacgagtatgataagcatgtaaatcaaatagaatcatattaggtgaggttactctgaagaacacaggttatcgctcggaaatgtcgagagacagtaacgagtaaagcagggattgtcgaattaaggctttcctaagataactagaatttttttcttacgtctcagaaagccaaagctcaattattgtgattaccctataatcttcttcttttattcggcgacctctttaatatgattaattggaggtttttaaattgaaagctgtcactgcatcatctaagaaaaataccctacttgctaaaagtatcgggaatcttaccttgctcatcattttaggcattttcatttttatcatcgtcttctcttggctaaaaatgaatcgccctctccacacccttccctcagaagaattcctcgcaacaccaagtaaaacagatgatttcttatctccatcaaatcttttttacttttcaattcgaaccatgtttcgaatgattgtggggatggcttggtccttcctgttttcctttgtttttggtattttagccgtaaaatataaaacggcacgaagagtcattttaccattagttaatttccttgaatctgttccattgctaggttttttgacctttacaactgcttggttacttggtttatttccaggaaatgtgatgggcgcagaagcggttgctatttttgccatcttcacaggtcaagctt

SEQ ID NO: 10 - DNA, 3480 bp, Enterococcus faecalis
  Designated as EF-7
aagcttctagcgtttcggattggcgcctatgatgcaccaggagagcgacgaatcaataccaaaaatatgcctacagcaggaggacttgcaatctacattgcttttgctagttcatgtttattgatttttcgttcgattatcccacaagattatatttggccgattattttggctggtggaatggttgttttgacaggcctcattgatgatattaaagagattactccaatgaaaaaaacaatcggtattttgttagcagcattagttattttattttgttgctggaattcggatagattttgtgacgttgccagttgttggaatgattgatttgcgctggtttagtttaccactaactttattgtggattttagcgattacgaatgcagtaaatttaattgatggtttggatggtttagcatcaggcgtatccattattggattaaccacgattggtattacagggtattttttcctacatgctaaaacggtctatatcccaattgttatttttattttagttgcgagcattgcgggatttttcccatacaatttttatccggctaaaatatttctaggagataccggggcgttattcctcgggtttatgattgcagtaatgtcgttacagggcttgaaaaatgctacgtttattacggtaattacgccaatggtgattttaggtgtgcaattacggatacggtttatgcaattattcgacggctattgaacaagaagcccatttcctcagcagataaaatgcatttacatcaccgcttgttatctttaggttttacccataaaggggcggtcatgactatttatgcattagcgttagttttttcctttgtctctttattgttcagctattcaagtacagtagcatcaattttattaattgtcttttgtttaattggcttagaactattcattgaactaatcggtctagttggcgaagggcatcaaccgttgatgtatttgttacggattttagggaatcgtgaatatcgtcaggagcaaatgaaaaagcgacttggcaagcattctaagagaaagtaaagaaatctttaggttgctttgcgagagctaaacctatgatataattccattaaacttaaaaaagtatatgtgtgaaacatatgctttttttttaagacgatgtttcagtagtaaggagaaatgagcatgcaagaaatggtaacaatctcgattgtcacttataatagtcgttacatttttaatgtactagaccaattaaaagccgaactaggtactgatagtatctatgatattcatatctatgacaatcattctgaaacagcgtatcttgaaaaattaacaacatatgaaccatttattactatccatcgcgctgaagaaaatcaagggtttggtcatggtcataatcaagtgttattcaatgcttcgacaaagtatgcaattatttttatcccgatgtgttggttactaaagacgtgcttgatcgttattagacgtatcaaatagataagaacattgcagtcggtagccctaaagttgttaaatgaagatggcacgacgcaatatttagttcgtcaaaaattagatgtcttcgattatatgttacgttttattccctttcaatttgtaaagaaaatttttgataaacgtttgagtatttatgaatgtcgcgatttgtcggatacagaaacaacggatattaaaatgggctcaggctgttttatgttgattgatcgtgaaaaattcgttgaaattggtgggttcgatgaacgtttcttcatgtactttgaagacaacgatttatgtttacgctttggcaaagcaggctatcggattctctatacgccttttgaaacggttgttcacatgtatgaaaagggcgcccataaaagtcgaaaattgtttaaaatctttatgcaatcaatggggaaattttttaacaaatggggctggaggttcttttaatgagtcaaagattagcggtagtcatcgtcttatatcaaatgaaaatggctgatacgccgaattatttgttattaaaagaagtggtagaccacccccaattgcacttatttatttatgacaacagtccacttcctcaagaagatgcattatttttacaaccaaatgttacttatcgacataatcctgataatccaggactagcgaccgcttataatgaagcgattgcttttagtcaagcgaatcaatgtgaattattgttgctccttgaccaagacacagaagtgccagcctcttattttgatacgttgatcatcatgccattagatccgactgtggcagtctatgttccaattgtagaagcaaatggacaacaaatttcgccagtatatagtgatcaatacgttgggcttaaaggagcaaagccaacagcagggatagccaaccaaccgttgatggctatcaattctggtacagttattacggcagaaacgctacgctggttggaaggattttcggaagaatttcctttggactatttagaccattggttcttttatcaattaaatcaagccaataaaaagattgaagtcttaccaatccacctaaaacaagaattgtctgttttagattatcgtacaatgagtcctcaacgttatcgctctattattgaagcagaaacgttattttatcgtcgatatgatcaagaaaagttttcccatcatcgacgccatttatttttacgcagtagtaagcaatttttaactgtcaaaaatcgccaaatttggcggcaaacattggcagaatttctcaagttaatgaaaggataatctatgatctcagtttgtattgcgacatataatggagaaaaatatctcgcggaacaattagatagtattcttttacaagtcagtgaagaagatgaactaattatttcagatgatggttctactgatcatacgttggaaattttgaggacgtatgcagcgaattatccccaaattcaattgttacaaggtcccagggcaaggagtgattgctaattttgcattttgccttacgcatacgaaaggcgaagtaatatttttagcagatcaagatgatgtttggttgccaaataaagtaacgacggtgacagaatattttgaagcgcaccctgacatccaagtggttattagtgacttgaaaattgttgatgcggatttacaagttaccaatccctcttatttaagtttcgaaaagtcaaaccagggttttggcgaaatgcgataaaaagtggctatattggggcaggtatggcctttcgtcaagaaatgaaaaacgtcattttacccattccgccagaagttcctatgcatgatatgtggattggcttattagctgcacggaagaagcaaacgggtctcattaaagaaccattagtgctttaccgaagacatggagcgaatgtcagccccattattaccaaaacaagtttccaacaaaaattaaattggcgtgtgaatttattaaaagctt

SEQ ID NO: 11 - DNA, 3615 bp, Escherichia coli
  Designated as EC-24
aagcttttcttgcgtgttcttgtgaggcttccttcgccattatcatcacgatccacataaataaagccgtagcgcttagacatttgtgaatgagatgcactgactaaatcaattggcccccaactggtgtaccccataatatccacaccatcggcaatcgcttcatttacctgtaccaggtgatcgtttaaataggcaattcgataatcgtcctgtatcgaaccatccgcttcaacgctgtcttttgcgcctaatccgttctcgacaataaataacggtttttgataacgatcccaaagcgtatttaacagaacccgtaatccaaccggatcaatttgccacccccactctgaacttttcagatgcggattggggatcatattcagtatgttgccctgcgcatttttattaatgctttcgtcgtgggaacacaaccagtcatgtataactaaagagatgaatcgacggtatgttttaaatctctgcgtcactttcagtcatctcaatggtgatattgtggtcgcggaagaaacgctgcatatagccgggatactggccacgcgcctgaacatcaccaaagaacatccagcgccggttctcttccatggcctgcaacatatcctgtggctggcaggtgagggggtaaaccagcccaccgagaagcatattgccgattttcgcttcggggagcaggctatgacaggctttaactgcccgcgcactggcaaccagttgatggtggatagcctgataaacttccgcctcgccactctcttctgccagccccacgcccgtgaatggcgcgtgtaacgacatgttgatttcattaaacgtcagccataacgccactttatgttggtagcgagtaaagaccgtgcgggcgtaatgttcgaagtgatcgatgaccgctcgattagccaaccgccgtagtttttcaccagcccatatggcatttcgtaatgggataacgttaccagcggcttgatccccgcctgcgccatttcatcaaacagccgatcgtaaaacgctaaccccgcttcattcggttcgacttcgtcgccctgagggaaaattcgcgcccaggcaatggaaatacgcagacaggtgaagcccatctcggcaaataacgcgatatcttccgggtaacggtgataaaaatcgatggcgacatctttgatattctctttccccaggatgcgcggttccatttttcccattacgcatgaggctgtaaatctgaggtcgagatccctttgccatcttcctgccaggcaccttccacctgattggcagctgttgcggcaccccaaagaaatgtttctggaaatgctttcataattaactccttttatcgttagcgaatgatggataacagcggttcacctgcgcttatctgcgccgtgccgtggggtaatacgtccgtaaaatcatcgctattactgattaataccggcgtcgtcagatcaaatccggcctcgcgaatagcagggatatcaaaagaaatcagccgatcgcctgtattgaccttgtcacccacgttgacgtgagcggaaaagaatttgccgtccagttttacggtgtcgataccgacatgaatcaggatctccacaccatcatctgactcaatgccaatggcgtgtaatgtggcgaacaacgaagcaattcgacccgcaaccggagaacgcacttcaccaaccgagggcagaatggcaatacctttacccaacaggccactggcaaacgtggtatcagcgacgtgaatgagcgacacaatctctcccgtcatcggtgaacagataccgccctgctcaggtggtgtaataacctctggtgttttctcttcggggcaccctgcgctggctgacgtttagcggtgatgaaatgaagcatcaccgtaccgacaaatgcgcaaccgatggcaatgacaccgccaataacgctggcccagacggtgaaatcaattcccgttgacgggatggtttgcatgaaggtgaaaatacttggcaaaccaaaggagtagactttcgtttgcgcgtagccaataatggtggcccccaaagccccactgatacaggcgataacaaaggggtacttacgcggcaggttgacgccatataccgctggttcggtgataccaaacagactcgtcaacgccgctgatcccgccaccacttttttctgcgcatcgcgttcgcagaggaagacgccgagcgccgccccgacctgcgccataatggcgggcattaacagcgggatcatggtgtcgtagcccagcacggtgaagttattgatacacaccggcaccaggccccagtgcagtccgaacatgacgaagatttgccagaagccgcccattaccgcgcccgcaaatgcaggaaccgcctgataaagccagagataaccggcggcaatcagttcgcttatccaggttgatagcggccccaccagcagaaaggtgacgggtgtgataaccatcagacatagcaatggtgtgaagaaatttttgattgccgacggtaaccacgcattaagtcggcgttccagaatgctgcacaaccaggcagaaaaaataatgggaataaccgatgacgagtaattcaacaatgtgaccggaatacccaggaaatccagccccagcgcatccgcttttgcgcgttctcgaaaagcagtacagaattaatggatgcactaacgctccaccaatcaccatggcagtaaatggattatcgccgaagcgtttccccgcggtgtatcccaggattatcgggaagaaccaaaacaaggcatcactggcgctgaataaaattaaataagtaccactttgttcgggcgtccactgaaaagtgagcgccagagccagcatacctttcaagatccccggttgcccgccatcaaaccgatacagaggcgtaaaaatacctgaaataacataaacaaagcggtttagacagattacctttatcatacattttccggtgcctgttgcgctttttcgtcaaggcctgccacactgttaaccgccaggaagacatcggccacatggttacctatgaccacctgaaactggccaccgctttccaccaccataataataccgggggtctttttcagtacctctgcttgcgctttgctttcatcctttaatttaaaaacgtaaatcgcgttgcgcaatgcatcagactcacaatgttatctgcgcccccgactcctgcgactatttttctggctaactccgtcataacttgccctctacgctttgcggcaaaactccaaaaaaaaacctgaaaaaaacggcctgacgtgaatcaagcaatttttttcaggttttgcccgcttagtgcggtaacaatcctttactcagtaataatatttcagtgttctttgcgcacgcgctctatatttatggctaaaaacataatctctgcgggtgaaattttacgttgatactgcaaaccaataaaaatggcgatccgttccgcacattgccatgcttgcgggtaattttgttttactgcttgttgtaatgattcatcactatcgttaattgaagcatgttcaagaatacgccaggataaaaacttcagatgtgtaaccagtcgctgataactcaagctt

SEQ ID NO: 12 - DNA, 4954 bp, Escherichia coli
  Designated as EC-34
aagcttaaccgctctcatctgttgaccgcacggcatagctatattctgccggtcctgggacgtagcgagattgacatgcaaaaaaacggtgcgcaggcggtaaccgttgaggattcaatgtcgatgattcatgcctcgcgtggcgtgttaaaacccgccggtgtaatgctgaaatcagagtgtgcagtggtcgcgggaatcgcgcaggcagcactaccccagagcgtggtagcctgggagtatctggtggaagattatgatcgcattcgcaatgacattgaagctgtgctgccagagttcgccgactataaccagcgcatccgtcatcccggtggttttcacctgataaatgcagctgctgaaaggcgctggatgacgccgtcaggtaaggctaatttcattaccagcaaagggctgttagaagatccctcttcagcgtttaacagtaagctggtcatggcgacagtacgcagccacgatcagtacaacacgacgatttatggtatggatgatcgctatcgaggggtattcggtcaacgagatgtggtctttatgagtgctaaacaagctaaaatttgccgtgtaaaaaacggcgaaagagttaatcttattgcgcttacgccagacggtaagcgcagtcacgccgcatggatagattaaaagtggtcatttaccctatggctgaccgctcactggtgacctattttccagaatcgaatcacatgctaacacttgataaccacgatccattaagtggcattcctggctataaaagtattccgcttgaattagaaccatcaaattaatgtctcttctcatttcttctgctgtcatccgcacagcagaagaattcctcattgactattatttcgcaatttgctcacatggattaaattaaactacatactataagatataaacttctgcctacagctgtaagaaactccgctcagtactgaagcaccagtcctatttcctcttttctccagcctgttatattaagcatactgattaacgatttttaacgttatccgctaaataaacatatttgaaatgcatgcgaccacagtgaaaaacaaaatcacgcaaagagacaactataaagaaatcatgtctgcaattgtgggtgtcttattactgacacttacgtgatagccattttttcggcaattgatcagctgagtatttcagaaatgggtcgcattgcaagagatcttacacatttcattatcaatagtttgcaaggctgtaaacaaacagcaaattataaatatgaaatgttaaaaaagtatcgataaaaactttattgttttaaggagataaaatgtcgctcgtttgttctgttatatttattcatcatgccttcaacgctaacattttagataaagattacgccttctctgacggcgagatcctgatggtagataacgctgttcgtacgcattttgaaccttatgagcggcattttaaagagatcggatttactgaaaataccattaaaaaatatctacaatgcactaacatccagacagtgacggtgcctgttcctgcgaagtttttacgtgcttcaaatgtaccgactggattgcttaatgaaatgattgcttatctcaactcggaagaacgcaatcatcataatttttcagaacttttgcttttttcttgcctgtctatttttgccgcatgcaaaggtttcattacactattaactaacggtgtgctatccgtttctgggaaagtgagaaatattgtcaacatgaagccggcgcacccatggaagctgaaagatatttgtgactgcctgtacatcagtgaaagcctgttgaagaaaaacttaagcaagagcaaacgacattctcacagattcttttagatgcaagaatgcagcacgcaaaaaatttgatacgcgtagaaggttcagtcaataaaattgccgaacaatgtggttatgccagtacatcttattttatttatgcgttccgcaaacatttcggcaacagtccgaagagagtttctaaggagtaccgttgtcaaagtcacacgggtatgaatacgggcaacacgatgaatgctttagctatttgattatttgctaacgagtagtcaaccacacacgctgcgtaagaattaaatggggcagccattccctgccccgcgttgtttttaggcgatatatttattgaaataaataagtgacatccatcacatatttatgcacttgcataacctgttgcatgattatttatgatctcaattctgcattttgtcagtaaaatgcaataatttattaaatatcaataaattagttgtttatcggcgagaaattacttaatagaacagaaagtaatgtcaacgctttatggactgttttttccctttttttagctaaatctgctatctctttatgtgactaacttcacttacatccacttatttctcttcgtaaaattactttggaattaagtacaataagaagaggaacatttatgaagtctgcattaaagaaaagtgtcgtaagtacctcgatatctttgatactggcatctggtatggctgcatttgctgctcatgcggcagatgatgtaaagctgaaagcaaccaaaacaaacgttgctttctcagactttacgccgacagaatacagtaccaaaggaaagccaaatattatcgtactgaccatggatgatcttggttatggacaacttccttttgataagggatcttttgacccaaaaacaatggaaaatcgtgaagttgtcgatacctacaaaatagggatagataaagccattgaagctgcacaaaaatcaacgccgacgctcctttcattaatggatgaaggcgtacgttttactaacggctatgtggcacacggtgtttccggcccctcccgcgccgcaataatgaccggtcgagctcccgcccgctttggtgtctattccaataccgatgctcaggatggtattccgctaacagaaactttcttgcctgaattattccagaatcatggttattacactgcagcagtaggtaaatggcacttgtcaaaaatcagtaatgtgccggtaccggaagataaacaaacgcgtgactatcatgacaccttcaccacattttctgcggaagaatggcaacctcaaaaccgtggctttgattactttatgggattccacgctgcaggaacggcatattacaactccccttcactgttcaaaaatcgtgaacgtgtccccgcaaaaggttatatcagcgatcagttaaccgatgaggcaattggcgttgttgatcgtgccaaaacacttgaccagccttttatgctttacctggcttataatgctccgcacctgccaaatgataatcctgcaccggatcaatatcagaagcaatttaataccggtagtcaaacagcagataactactacgcttccgtttattctgttgatcagggtgtaaaacgcattctcgaacaactgaagaaaaacggacagtatgacaatacaattattctctttacctccgataatggtgcggttatcgatggtcctctgccgctgaacggggcgcaaaaaggctataagagtcagacctatcctggcggtactcacaccccaatgtttatgtggtggagaaggaaaacttcaacccggtaattatgacaagctgatttccgcaatggatttctacccgacagctcttgatgcagccgatatcagcattccaaaagaccttaagctggatggcgtttccttgctgccctggttgcaagataagaaacaaggcgagccacataaaaatctgacctggataacctcttattctcactggtttgacgaggaaaatattccattctgggataattaccacaaatttgttcgccatacagtcagacgattacccgcataaccccaacactgaggacttaagccaattctcttatacggtgagaaataacgattattcgcttgtctatacagtagaaaacaatcagttaggtctctacaaactgacggatctacagcaaaaagataaccttgccgccgccaatccgcaggtcgttatagagatgcaaggcgtggtaagagagtttatcgacagcagccagccaccgcttagcgaggtaaatcaggagaagtttaacaatatcaagaaagcactaagcgaagcgaaataactaaaccttcatgcggcggatttttccgccgccttattgagcgagatagcgatgcacgttacagccaagccctccagttttcaatgtaatctcaaatgtgattactgtttttaccttgaaaaagagtcgcagtttactcatgaaaaatggatggatgacagcactttgaaagagttcatcaaacaatatatcgcagcgtctggcaatcaggtctattttacctggcaaggcggtgaacccactctggctggcctggattttttccgtaaagttattcactatcaacaacgctatgcaggccaaaaacgtatttttaatgcattacaaacgaatggcattttattgaataatgaatggtgtgccttctcaaagaacatgaatttctggtggtatctcgatcgatggcccccaggagttacatgaccgttacagacgcagtaattcaggtaacggtacttttgcaaaagtgatagcagccatcgagcgtctgaaatcatatcaagtagagtttaatacgttaaccgtcattaataacgttaatgtccattaccctcttgaggtttatcattttttaaaatctatcggcagtaaacatatgcaatttatcgaattgctagaaaccgggacgccgaatattgatttcagtggtcatagtgagaacacattccgtatcattgatttttctgtgcctcccacggcttatggcaagtttatgtcaaccatttttatgcaatgggttaaaaacgatgtgggtgaaattttcatccgtcagtttgaaagctt

SEQ ID NO: 13 - DNA, 3796 bp, Escherichia coli
  Designated as EC-39
aagcttaatcgcgtgaatcaggagtaaaaaaatgacaacccagactgtctctggtcgccgttatttcacgaaagcgtggctgatggagcagaaatcgcttatcgctctgctggtgctgatcgcgattgtctcgacgttaagcccgaactttttcaccatcaataacttattcaatattctccagcaaacctcagtgaacgccattatggcggtcgggatgacgctggtgatcctgacgtcgggcatcgacttatcggtaggttctctgttggcgctgaccggcgcagttgctgcatctatcgtcggcattgaagtcaatgcgctggtggctgtcgctgctgctctcgcgttaggtgcgcaattggtgcggtaaccggggtgattgtagcgaaaggtcgcgtccaggcgtttatcgctacgctggttatgatgcttttactgcgcggcgtgaccatggtttataccaacggtagcccagtgaataccggctttactgagaacgccgatctgtttggctggtttggtattggtcgtccgctgggcgtaccgacgccagtctggatcatggggattgtcttcctcgcggcctggtacatgctgcatcacacgcgtctggggcgttacatctacgcgctgggcgacaacgaagcgacaacgcgtctttctggtatcaacgtcaataaaatcaaaatcatcgtctattctctttgtggtctgctggcatcgctggcgggatcatagaagtggcgcgtctctcctccgcacaaccacggcggggactggctatgagctggatgctattgctgcggtggttctgggcggtacgagtctggcgggcggaaaaggtcgcattgttgggacgttgatcggcgcattaattcttggcttccttaataatggattgaatttgttaggtgtttcctcctattaccagatgatcgtcaaagcggtggtgattttgctggcggtgctggtagacaacaaaaagcagtaataacgactacaggcacatcttgaatatgaacatgaaaaaactggctaccctggtttccgctgttgcgctaagcgccaccgtcagtgcgaatgcgatggcaaaagacaccatcgcgctggtggtctccacgcttaacaacccgttctttgtatcgctgaaagatggcgcgcagaaagaggcggataaacttggctataacctggtgctggactcccagaacaacccggcgaaagagctggcgaacgtgcaggacttaaccgttcgcggcacaaaaattctgctgattaacccgaccgactccgacgcagtgggtaatgctgtgaagatggctaaccaggcgaacatcccggttatcactcttgaccgccaggcaacgaaaggtgaagtggtgagccacattgcttctgataacgtactgggcggcaaaatcgctggtgattacatcgcgaagaaagcgggtgaaggtgccaaagttatcgagctgcaaggcattgctggtacatccgcagcccgtgaacgtggcgaaggcttccagcaggccgttgctgctcacaagtttaatgttcttgccagccagccagcagattttgatcgcattaaaggtttgaacgtaatgcagaacctgttgaccgctcatccggatgttcaggctgtattcgcgcagaatgatgaaatggcgctgggcgcgctgcgcgcactgcaaactgccggtaaatcggatgtgatggtcgtcggatttgacggtacaccggatggcgaaaaagcggtgaatgatggcaaactagcagcgactatcgctcagctacccgatcagattggcgcgaaaggcgtcgaaaccgcagataaagtgctgaaaggcgagaaagttcaggctaagtatccggttgatctgaaactggttgttaagcagtagttttaatcaggttgtatgacctgatggtgacataaatacgtcatcgacagatgaacgtgtaatataaagaaaagcagggcacgcgccaccctaacacggtggcgcattttatggacatcccgaatatgcaaaacgcaggcagcctcgttgttcttggcagcattaatgctgaccacattcttaatcttcaatcttttcctactccaggcgaaacgtaaccggtaaccactatcaggttgcatttggcggcaaaggcgcgaatcaggctgtggctgctgggcgtagcggtgcgaatatcgcgtttattgcctgtacgggtgatgacagcattggtgagagcgttcgccagcagctcgccactgataacattgatattactccggtcagcgtgatcaaaggcgaatcaacaggtgtggcgctgatttttgttaatggcgaaggtgagaatgtcatcggtattcatgccggcgctaatgctgccctttccccggcgctggtggaagcgcaacgtgagcgtattgccaacgcgtcagcattattaatgcagctggaatcaccactcgaaagtgtgatggcagcggcgaaaatcgcccatcaaaataaaaactatcgttcgcttaacccgctccggctcgcgaacttcctgacgaactctgcgctgtggacattattacgccaaacgaaacggaagcagaaaagctcaccggtattcgtgttgaaaatgatgaagatgcagcgaaggcggcgcaggtacttcatgaaaaaggtatccgtactgtactgattactttaggaagtcgtggtgtatgggctagcgtgaatggtgaaggtcagcgcgttcctggattccgggtgcaggctgtcgataccattgctgccggagatacctttaacggtgcgttaatcacggcattgctggaagaaaaaccattgccagaggcgattcgttttgcccatgctgccgctgcgattgccgtaacacgtaaaggcgcacaaccttccgtaccgtggcgtgaagagatcgacgcatttttagacaggcagaggtgacgcttggctacaatgaaagatgttgcccgcctggcgggcgtttctacctcaacagtttctcacgttatcaataaagatcgcttcgtcagtgaagcgattaccgcaaagtgagcgcgattaaagactcaattacgcgccatcagctctggcgcgtagcctcaaactcaatcaaacacataccattggcatgttgatcactgccagtaccaatcctttctattcagaactggtgcgtgtcgttgaacgcagctgcttcgaacgcggttatagtctcgtcctttgcaataccgaaggcgatgaacagcggatgaatcgcaatctggaaacgctgatgcaaaaacgcgttgatggcttgctgttactgtgcaccgaaacgcatcaaccttcgcgtgaaatcatgcaacgttatccgacagtgcctactgtgatgatggactgggctccgttcgatggcgacagcgatcttattcaggataactcgttgctgggcggagacttagcaacgcaatatctgatcgataaaggtcatacccgtatcgcctgtattaccggcccgctggataaaactccggcgcgctgcggttggaaggttatcgggcggcgatgaaacgtgcgggtctcaacattcctgatggctatgaagtcactggtgattttgaatttaacggcgggtttgacgctatgcgccaactgctatcacatccgctgcgtcctcaggccgtctttaccggaaatgacgctatggctgttggcgtttaccaggcgttatatcaggcagagttacaggttccgcaggatatcgcggtgattggctatgacgatatcgaactggcaagctt

SEQ ID NO: 14 - DNA, 6914 bp, Enterobacter cloacae
  Designated as ET-49
aagcttttcgagttcgccatccggcaacagctcactgagcttttacgcgcccagggtgcctttgaactcaattcccagctcagtaaggcggtcctgaataatctctttgcgagatttttcactggtaccggcatcaggtgttgcaggtttcagctcgccaccagcctcgcccttcatcagccggacgttagacttcagcgccgggtgaagatctttcaactccaccacgtcgccaacctttacgccgaaccatgggcgcacaacttcgtatttagccatgctgtttccttacgccaggttagcgccgtagacaacgccagacaggcctgatcgtctgcagtaatttgcaggccttcagcagacatgatctggaagttgtagttaacgttaggcagtgggcgcggcagtggcacaacgccaacagccatacccaccagtggggagatcacgtcacgacgacgaacgtacgcgataaactcgttaccggtcagcgcgaagtcatgcggatttctttcaccggtgcgaatggcagaacagcctgcaggagagtgccgctcaccacaccattaactacgtatggctgagccatatttgcccagatctcaggggaaacccacatcacatcatactgagctactttgttggtgcgtgcggtggtaccgaatgctcctttaccaaagaactcaaaatattgagtcgtggttgcgctggtcaggtcgatgttcgcaccaccagcaccagaaccgaggttaatcttcttggtgttgcggtggttcttgatgccctgcgccgggtaggactgaacctgaatttttgaatcgccgttcaggtagtagttgacgcgcttctggttgaacttgcgcatcttcgccatctgcgaatccagaaccagatcaatgcctacagagttaaggccagcagcatgacgccagttaacaccgtagccagcagtgaacaccggaatcgggtcgccatcgctcgcgtagtcagtgtggtcgaaggagaatggcgcctgaccatcgatgcttactgacacgtcgtcagcgatgtcgccgaccacgttatacagcttggcggttttaccaaccggcagcacggtctgaacgccgatcaggtcgtttacgatttccatgccaacttcctgatcccgcagctgcagcacctggttgtcaatctcagcccagaagtcacgggagaaaccgccaacagcgttacaagccagcatgtcaggcgtcatcattgcgcggttagctgcaatgatggaatcgttctgtaggttccacatgttgcggtttgcccacagctcactccagtgcccgccgaggcgggagttagtcgccagcgtctctttagagaagtacatatgtgtttgtccttttgttacgcgccagctgcggcgacagtgccaacgcgcatacgcacgcgaatgaagtcagtggtgctggccgcgatggtgtattcatcctggctgtagccgatcactgaatcagtgtcggatgtggcaagggtaaactgaccggcagttcccagcttgatcgggctgtcttttttatacgcaccaggcaggcagcgcagcgccagctcacgaccttcttcgacgtagttacctactgccgaatccccggcagggatttcttcggtgattgtcaggccctggtgataaccgacatcgatgatgtacaggcggccggttagcgcggtggcctgagcgaatttatcggatgagttgatggttgcggcggtgccaggaagcaacccggcggccgttgtgcgggtttcggtcttgtacagagactgaccgtcgatattaacgcgacgataacgtggcattattccggctccttacttgaagtgttcgtctgcggctggtgcgccggtttctttgtgctgctgagcattgttggtgcccagcgacttgaacatcgcgtccagagcttcgcctgacagagcgttcgcgagcgatatcgccatggaccttcgcaaccgcttcgcgctttgctttctcttcggcacgggagttcgcggtaagggtttccgcgagttgcttctgattggcctgcagcgcatcaaccttttccgcgagaggcttaatagccgcttcagtattggtcgcaacagcctggccgatcatgctgccgatttgttccagttcttctttggttaaaggcatgtcgcctccgttttgtggtttggtgcaggctgttcctgcggtgtgaatagagctttgaattgttagcgacgactgccacccacgactcctggcgcgctactgcggttccggtatcgtcgattgtgatcttcccgccatcagcgaataccgtaaacctgagcatcaccgccatttcgcacgatgaccacctgcgagtcagtgagtcagcaacccaggcatattcatccgtgcccggcgcaaacttggctttggctgcccgatcgagacgctgctcgcgctcccggtaggattcacccaccagcgcgccggagttcgctttaagcggctgcgccagatcggcgtttaccatcaggccaacgccctgctcaggggtggcggctccgacttcgtgcagtaggatcgcgtcgtggtccatgctgtgaatcttcgccacccactcggcacccgtagctctctgttgttcgttaggctcaagctggtcgaggaaagcggcgacactggtatgaatcggcggaacgtcatcgccgcgctcgatggctgcgacgcgctcaagtagttctcggccaccttcagactcaccggcgcgggcaacatcaacccacttttcgaggtagatacgattaccggacttcttaacgttgcggttccacgcgccgatatggcctgcgttaatcccctccggggagaaagcagacacgaactgaccattaacctgagggtggcccagcggcgccagggtaccttccagccccttatagtgggcgtcgatttgctcttgcgtgtacaagccgccattcatgacgacgttagctggaagtgtgtagctcggcagcaccaggtgctcacgcccgttgtatgtttcgcgccggatagactggctgttcacctttgtggtgatgttgacctgaatatgctcaccatgtttcggtgcctggattggacgctgtgcttcgtggtttacctggaatttcatgagttatttctccgcccaggcgtaaccgctcgcctgcatcgatttatattcctgtttgagtttcgtgatggtgtccgggtattccggcttgccgtccgcatccaccagcaccgactgctggctgcatttgcagttgatggagttgccatctttgctgtaccagtcacgcacctcttcgttggtgtagaggtgggcatgggcgcactgcgtgggtatgtcgcgttgtcggcgacagagctgagatgtgaaccagcagcgttttaaggccgaacaggtcattcgcctcttggtcttcatcccacttggcccggcgcagcgcggtagtcacttcagtgcgtgctatccggttagcccggcgtttctcgatgccggtctggtctgtcaggttgcgggcaatgtccagaggattgagcccgcgcccaacaccatcagtaagacacgcgccatgtcgcgcttaacgtcagccgtcagccccttcatttcctcaaatacacgcgcatgcaccagcgccatgcgtttctgatactggtcgcttgcgaggatggaggccagcgactcacgcccggctgcgtacaccggggattgctgactgaggttgtagaacgactgcccggtccctttttccgaagccagatcgatgtactcgtaaaaccacaggtcgtaatcgccaccttcaagcagtacctgatcaaccaggtaactggcatcgttcaggatgatggagagtagcattgggtttagctggtattcgtatctggcgtttactgcgagggaggaaggtattttgttgagtgctgatttgtacgccttgccaatcttattcatccgcctggcgaagtctttcattgcccggcgttccagcgcatcggctccggtcggatcctgatagttacgcggcagaatcggtggcttcgtcttcttcgtcgccatcctcttctcctaatggaaattcatcgacgttttcataaccggcagcagtgcggaatttcttcacgactaaaggctggtttttctccgctcccctggaacgtctggttaatctctgccatggttttggcatttgcgagtttctcagttccagtctgttcgttgaggtcatcccagataaccgtcttctcgctgactgcatcaataattttcaggtcgatgagcttgtcactgaagtcttcaatttcgaatgacaggtcaccgcgccgtgactggcagcgcgcgttgaaatatttctgatcctcggtgcttgccctttcacccgtctgcatcccaaccagaaccttcacagggatatcaacagatgcagcgaaggtttgcaggttgacgttataggtcgctgacggatccgctacagctgtgaccagtggtgtgactgtagccccttgggttgtcatcagaacatcgttaccacggttcatttccccggcaacttcgttaaacttatcctgcaactcgtccatgtcacgccataaagtgacgcgagattgttgaaatcgatttccttctcaaagttgacattaagctgccgcgcggcgttctttaggaatgactcaccagaaccaccctcgaccttctcaaggctgacgcaggcgttatagccaggctcaaggaagccaatagcatcattagaatagtcaccaaggataaagacgcgatcgggatgtacgaagcgctgattagttccaccgcttggaaggctctcaacatatttccactgctttggctgcccgtagcctgccgatttctggtcagttacccactcgctgactgttaatgacccagcccatgcgatcgtaaccttttttagtgacttgccacgaacaacaggctgatcccatgttctggaatcattgatatgcagcaggatacccgcataacgtccgacctgtcggcggcggtctgcttcagcaaaagcccgccaaaggcgctttgtgaaaacctttttggtgttcttctcccaggcagtttcatccttactctcgtcggcatcatcaccctcgatgatttccgggttggtctgccagcacttgcccaccagcttctctactgcgccgtgggctattccaccgcgacgatacagtgcgtagaggttttcgtaagtgacctgctcagggaatccatactcgcaccatgcggaatggcgcttattgtccagccccattgtaggcgccaacagccccatacgggcacgggccatccgcgcatcgttcaacgcatggttgacggcgagagttaatttgtcagtcatggtttgtccgttggtggatttaaggcataaaaaaaggccgctttggcgaccttgtggctatttaaaaagctaaactctgttgaacgaaataaacataatctgctcaggcttaacgccataatcacttgccaacttctgagtgcactcaattaagacagttgatgcagatttcgaagagcttgcaccataaatttcgaagttttcaaatactccgccgttggtgtggtaaatcttatatgacataaaccaatcattcataatatctactcccttacagaattgagtagatattatcggcaagtgcatatgtttctttaaattatctcaaccttttcgggatcatcatcccggccatctggcccttacgtttaatgtgtccgtcgaggctgtagcgaataccgtcccagcagtgttcgtaaccgtctgccagtttaggcaatacctcgccggtgatgcggtccgttttgtaggaccacatgcgggcctctctcgccacattcttgcagcgaggatggataatgatttcgtcaaagccgcgaagatgcgcgataccgtcctcaacactcccctgccatttctcggcagccgagatgttgaagccctggcgcttgagatagctgatagtctcgggtcgggcggagtcggccttgatgggccagtcacgcgatccggggattgtgtcgtatagctctggcatatggtcgagctctgtctgctgaccgtatgcctcgtattcgatgtacagccggttgtgcaggatgaacgagcgcaccagcgtgttagggtctttggcgaaaccgaagtcagcaccgaagaaaaggcgatcggcctctttccatagctggtccgagaactcagcgatccggtatttaccggccagcacctgcttatcagagttttcgaggtaagcaccttcccaaacccacgcgtatgttgccgggtcaaggcggcgctgatcgttctgtcgctcaccttccagcacgtcggggaaccatggattatccgtgtagttcatctcaacgtgatacagtcgtcgccagcctctttacggaaacgcttatccgtgcgctgccgtcgcgctccgggttccatgtcacccaaatctctgaaccttcctcacgaacggtcgggctcagcttctgccaggctatttcgctgactgattcagcctcatcaacccaacagagcaagatgcgcgctttcgacttgatgctgtcgaggttatgccgcagaccgcagaacacgtagttaacgctcttgtcgatggtgcggatgtacttctcgccgatatcaaagttggaagccagccagggaacagacaggatagcctgtttcacctcctgcatactcgactcttccagtgagttcatgaattcacgcgcacagagcaccacgccgctttcaccgttcatcatcgactgatacgcctttacggctgtcatcagcgcaaaagtgcgcgtcttggcactaccacgcccaccatgcgagcaccggtaacgcttattctcggcgatgaacagtggcgcaagctt

SEQ ID NO: 15 - DNA, 5975 bp, Klebsiella pneumoniae
  Designated as KI-50
  misc_feature (456)..(456): n = a or t or c or g
  misc_feature (3074)..(3074): n = a or t or c or g
  misc_feature (4180)..(4180): n = a or t or c or g
  misc_feature (4288)..(4288): n = a or t or c or g
  misc_feature (4290)..(4290): n = a or t or c or g
aagcttattccacgctggaggcgtccgggattatcggcgtcaacgctatcgccggcatcgccgggaccatcatcgccggcatgctctccgaccgctttttcaaacgcaaccgcagcgtgatggccggattcatcagcctgctgaacaccgccggcttcgccctgatgctctggtcgccgcacaattactacactgatattctggcgatgattatcttcggggccaccattggcgctctgacctgcttccttggcgggctgatcgccgtcgatatctcttcgcgcaaggccgccggggccgcgctcggcaccatcggcatcgcagctacgccggcgccggcctgggcgagtttctcaccgggttcattattgataaaacggctatccttgaaaacggcaaaacgctgtatgatttcagcacgttggcgctgttctgggtgggtacggtctgggttcngcgctactctgttttaccactgccgccatcgtcgcccggcgccatgccgtcgaacggcagacctcgttctcctcataaccgattaacgaataaggaagaagatatgatgcctgcaagacatcaggggctgttacgcctgtttatcgcctgcgcgctgccgctgctggcgctgcaatctgccgccgccgcggactggcagctggagaaagtggtcgagctcagccgccacggtattcgtccgccgacggccggcaaccgggaagccatcgaggccgccaccggccgaccgtggaccgagtggaccacccatgacggggagctcaccggccatggctatgccgccgtggtcaacaaagggcgtgcggaaggccagcattaccgccagctcggcctgctgcaggccggatgcccgacggcggagtcgatatacgtgcgcgccagcccgctgcagcggacgcgagcgaccgcccaggcgctggtggatggcgccttccccggctgcggcgtcgctatccattatgtcagcggggatgccgatcccctgtttcagaccgacaagttcgccgccacgcaaaccgaccccgcccgccagctggcgcggtgaaagagaaggccggggatctggcgcaggtcggcaggcgctggcgccgaccatccagctattgaaacaggcggtttgtcaggccgataagccctgcccgatcttcgataccccgtggcaggtcgagcagagcaaaagtgggaagaccaccattagcggactgagcgtgatggccaatatggtggagacgctgcgtctcggctggagtgaaaacctgcctctcagccagctggcgtggggcaagatcacccaggccaggcagatcaccgccctgctgccgctgttaacggaaaactacgatctgagtaacgatgtgttgtataccgcgcaaaaacgcgggtcggtgctgctcaacgctatgctcgacggcgtcaaaccggagcgaatcgaacgtacgctggctgctgctggtggccatgacaccaatatcgccatggtgcgcacgctgatgaactttagctggcagctgccgggctacagccggggaaatatcccgccgggcagcagcctggtgctggagcgctggcgcaacgcgaagagcggagaacgctatctgcgggtctatttccaggcccagggcctcgacgacctgcgtcgtctgcagacgccggacgcgcagaccccgatgctgcgtcaggagtggcatcagccgggctgccgtcagaccgatgtcggtacgctgtgtcccttccaggcggctattaccgccctcggtcagcgtatcgaccgatcatccgccccggcggtagcatggtcctgccgtagcggcgcggtgtttgtccgggcccgggaaaacctttttttccaggccggcacgacgtccgttatccgttgtccggcgcaaacgccccggcggcgacctgcgccggggtgacacccgctgtccagcacccagccgcttatcagcccagcaggcgtgacgtcgaacgccggattgtaaacggtggcccccgtcggcgcccactgtaccgcgccgaagctgcccgccactccggtcacttccgccgccgcgcgctgctcaatggggatcgccgccccgttcgggcaatggcggtcgagggtggtctgcggggcagcgacgtaaaacgggatctggtgataatgggccaaaaccgccagagaataggtgccgattttattcgccacgtcgccgttggcggcgatacggtcggcgccgacccacaccgcatccacctgcccctgcgccatcaggctggcggccattgaatcggcgatcagctgatagggcacgcccagctcgcccagctcccaggcggttaaacgaccgccctgcagcagcggccgggtttcatcaacccatacgttggtcacttttccctgccggtgcgccagcgcgataacgccgagggcggtccctaccccggcggtcgccaggccaccggtgttgcagtgggtcagcagtcgactgccgggcttcaccagcgcactgcccgcctcagcgatgcggtcgcacagctgtttatcttcttcgaccagacgcaaggcttccgcttccagcgcctgcgggtaatctccgggccagcgctgcttcatgcgatcagattattcatcaggttgaccgccgtcggccgcgccgcgcgcagtctccagcgcctgctggagtgcatcccggttcaggccgcgctgggccagcagggccagcagcaggctggcggacaggccaatcagcggcgcgccgcgcaccccgcaggtatgaatatggtccaccagcagcgcaacgttatccgccgccagccagcgtttttcctgcggcaaggcctgctggtcgagaataaaaagctgattttcactcacccgcaggctggtggtctgtaatgtctgcatgtcgttaaatccctgttgcgttgttgtatcacattgtgtcaggatggaatccagaagtatagacgtctgaacggcttaatcagaattcgaggatcgaggcaatgtcgcaataccataccttcaccgcccacgatgccgtggcttacgcgcagagtttcgccggcatcgacanccatctgagctggtcagcgcgcaggaagtgggcgatggcaactcaatctggtgtttaaagtgttcgatcgccagggcgtcacgggcgatcgtcaaacaggctctgccctacgtgcgctgcgtcggcgaatcctggccgctgaccctcgaccgcgcccgtctcgaagcgcagaccctggtcgcccactatcagcacagcccgcagcacacggtaaaaatccatcactttgatcccgagctggcggtgatggtgatggaagatctttccgaccaccgcatcttgcgcggagagcttatcgctaacgtctactatccccaggcggcccgccagcttggcgactatctggcgcaggtgctgtttcacaccagcgatttctacctccatccccacgagaaaaaggcgcaggtggcgcagtttattaacccggcgatgtgcgagatcaccgaggatctgttctttaacgacccgtatcagatccacgagcgcaataactacccggcggagctgggaggccgatgtcgccgccctgcgcgacgacgctcagcttaagctggcggtggcggcgctgaagcaccgtttctttgcccatgcggaagcgctgctgcacggcgatatccacagcgggtcgatcttcgttgccgaaggcagcctgaaggccatcgacgccgagttcggctacttcggccccattggcttcgatatcggcaccgccatcggcaacctgctgcttaactactgcggcctgccgggccagctcggcattcgcgatgccgccgccgcgcgcgagcagcggctgaacgacatccaccagctgtggaccacctttgccgagcgcttccaggcgctggcggcggagaaaacccgcgacgcggcgctggcttaccccggctatgcctccgcctttctgaaaaaggtgtgggcggacgcggtcggcttctgcggcagcgaactgatccgccgcagcgtcggactgtcgcacgtcgcggatatcgacactatccaggacgacgccatgcgtcatgagtgcctgcgccacgccattaccctgggcagagcgctgatcgtgctggccgagcgtatcgacagcgtcgacgagctgctggcgngggtacgccagtacagctgagtgcgcctgtttccctcaccccaaccctctcccacagggagagggagcaccccctaaaaaagtgccattttctgggattgcccggcgngntgcgcttgccgggcctacagatagccgcataacggtttgatcttgcactctttcgtaggccgggtaaggcgaaagccgccacccggcagacatgcgagtacaattttgcatttaccttaccctcaccccagatactcaatcaccgatagcccgccgttgtaatcggtgctgtagataatgccttgcgcatcgacaaacacgtcacaggactggatcacccgcgggcggccgggacgggtatccatcattctctcagcgcagccggcaccagcgccccggtctccagcgggcgatacgggttggaaatgtcgtaagcccgcacgccggcattctgatacgtggcaaaaatcagcgttgagctgacaaagctccccggccggttctcatgcaggttgtgcggaccgaaatgcgcccctttcgccacgtaatccgcttcatccggcggcgggaaggtggcgatgctcaccgggttggttggctcgcggatatcaaacagccagatcagcttctcgccgtcctcctggttatcgagcaccgcttcatccagcaccaccagcagatcgcgatccggcagcggcagcgcggtatgcgttccgccgccgaacggcgggctccagttgcgatggctaatcagcctcggctgggtacggtctttgacatccagcagcgtcaggccgccgtcgcgccagctgcgtaggcgtatccccggcaataatggcgtgatgcagcgcatagcgtttgccctgcggccagtccggtgtttcaccgcccgcctggtgcatccccggcagccaccagcgcccggctacttcgggcttacgcggatcggccagatcgatggtcaggaagatgtagtcggtaaaaccgtcgatcagcgcagacacatacgcccagcgcccgccgacgtaccagatgcggtgaataccgatgccgttaagcgacaggaaactgatttcccgcgctgcgcgggagtggaaatatcaaagatgcgcagcccggcgctccagcccctgtcctgcacatcgctgaccgtgtcacccaccgagcgggtgtagtacaccttctcatcagcaaaacgggcgtcagcaaacagatcccgggcgttgatcaccagcagcagatcgtcatgcgcctggagtgcacgttccaggtgcccggcggcgcggcaatatagttgacggtggtgggccgggtcggatcgcgaacatcgaccacggaaaaaccctgcgacaccatatggccgatataggcgaatccgcggtgcaccatcagctgcacgccgtccggacgaccgccctgatcgctatggccaatcagccgcatattgcggctgtattcgggggaaggtaatgctgacataggggatccctctcgcccggtggcatggttttcccccctctcctgcggagagggccggggcgagggcaccaggccgccgcccaccgccacccggcttgattttatttgttcttcgcttccagcgtcgcgaaccacggcgcgataaagtcttcggtctggccccagccagggataattttccccagcgacgccacgtttaccgctcccggctgggccgccagcagcgcctggggaatcgctgccgccttgaagtcgtaggtggctggcgtcggctcgccggcgatcttgttggcgatcagccgcacgttggtcgcgccgataagctt

SEQ ID NO: 16 - DNA, 899 bp, Candida albicans
  Designated as CA-26
gaattcctagtaagcgcaagtcatcagcttgcgttgattacgtccctgccctttgtacacaccgcccgtcgctactaccgattgaatggcttagtgaggcctccggattggtttaggaaagggggcaacctcattctggaaccgagaagctggtcaaacttggtcatttagaggaagtaaaagtcgtaacaaggtttccgtagtgaacctgcggaaggatcattactgatttgcttaattgcaccacatgtgtttttctttgaacaaacttgctttgcggtgggcccagcctgccgccagaggtctaaacttacaaccaattttttatcaacttgtcacaccagattattacttaatagtcaaacttcaacaaacggatctcttggttctcgcagcgaaatgcgatacgtaatatgaattgcagatattcgtgaatcatcgaatctttgaacgcacattgcgccctctggtattccggagggcatgcctgtttgagcgtcgtttctccctcaaaccgctgggtttggtgttgagcaatacgacttgggtttgcttgaaagacggtagtggtaaggcgggatcgtttgacaatggcttaggtctaaccaaaaacattgcttgcggcggtaacgtccaccacgtatatcttcaaactttgacctcaaatcaggtaggactacccgctgaacttaagcatatcaataagcggaggaaaagaaaccaacagggattgcctcagtagcggcgagtgaagcggcaaaagctcaaatttgaaatctggcgtctttggcgtccgagttgtaatttgaagaaggtatctttgggcccggctcttgtctatgttccttggaacaggacgtcacagagggtgagaatcccgtgcgatgagatgacccggg

SEQ ID NO: 17 - DNA, 189 bp, Candida albicans
  Designated as CA-26-1
gcgcaagtcatcagcttgcgttgattacgtccctgccctttgtacacaccgcccgtcgctactaccgattgaatggcttagtgaggcctccggattggtttaggaaagggggcaacctcattctggaaccgagaagctggtcaaacttggtcatttagaggaagtaaaagtcgtaacaaggtttccgta

SEQ ID NO: 18 - DNA, 224 bp, Candida albicans
  Designated as CA-26-2
gctgggtttggtgttgagcaatacgacttgggtttgcttgaaagacggtagtggtaaggcgggatcgtttgacaatggcttaggtctaaccaaaaacattgcttgcggcggtaacgtccaccacgtatatcttcaaactttgacctcaaatcaggtaggactacccgctgaacttaagcatatcaataagcggaggaaaagaaaccaacagggattgcctcagt

SEQ ID NO: 19 - DNA, 369 bp, Candida albicans
  Designated as CA-26-3
aaacggatctcttggttctcgcagcgaaatgcgatacgtaatatgaattgcagatattcgtgaatcatcgaatctttgaacgcacattgcgccctctggtattccggagggcatgcctgtttgagcgtcgtttctccctcaaaccgctgggtttggtgttgagcaatacgacttgggtttgcttgaaagacggtagtggtaaggcgggatcgtttgacaatggcttaggtctaaccaaaaacattgcttgcggcggtaacgtccaccacgtatatcttcaaactttgacctcaaatcaggtaggactacccgctgaacttaagcatatcaataagcggaggaaaagaaaccaacagggattgcctcagt

(19) United States
(12) Patent Application Publication
(10) Pub. No.: US 2007/0059687 A1
(43) Pub. Date: Mar. 15, 2007

(54) Process for evaluating phagocytotic function and use thereof

(76) Inventors: Tsuneya Ohno, Tokyo (JP); Akio Matsuhisa, Osaka (JP); Hiroyuki Keshi, Osaka (JP); Kanako Abe, Osaka (JP); Norihiko Sugimoto, Osaka (JP); Hiroshi Ueyama, Osaka (JP); Soji Eda, Kyoto (JP); Hirotsugu Uehara, Hyogo (JP); Takahisa Iwami, Osaka (JP); Seiji Yamamoto, Osaka (JP); Hiromasa Araki, Nara (JP)

Correspondence Address: David A. Gass;Marshall, Gerstein & Borun, 233 S. Wacker Drive, Suite 6300, Sears Tower, Chicago, IL, 60606-6357, US

(21) Appl. No.: 10/479,027

(22) PCT Filed: May 27, 2002
(86) PCT No.: PCT/JP02/05106

§ 371 Date: Sep. 10, 2004

(30) Foreign Application Priority Data
May 31, 2001 (JP) 2001-165954

(51) Int. Cl. C12Q 1/68; C12N 5/08

(52) U.S. Cl. 435/6; 435/372; 435/325

(57) ABSTRACT

A digested phagocyte prepared by contacting in vitro a phagocyte with a foreign microorganism and isolating the phagocyte so contacted; a process for producing the same; and a process and a kit in which these are utilized are disclosed. An experimental model, which enables in vitro evaluation of a phagocytotic function of phagocytes, is provided.

### TECHNICAL FIELD

The present invention relates to a process for evaluating a phagocytotic function, and more particularly, provides an experimental model of an infection which is useful in diagnosis of an infectious state by a foreign microorganism, such as for example, sepsis and the like by bacteria, fungi or the like, or in development of therapeutic drugs for an infectious disease. In addition, the present invention also relates to a process for detecting, identifying and evaluating phagocytotic ability and/or germicidal capacity by a phagocyte; a process for the determination of an effect of a modulator of a phagocytotic function; a process for screening a modulator of a phagocytotic function; and a kit for putting any of such processes into practice.

### Background Art

Infectious diseases and sepsis are often caused due to the underlying disease which had been presented previously, through infection by an attenuated microorganism. Although such a state may frequently occur in a clinical scene, ideal animal models which cover all of the clinical symptoms have not yet established. Factors for such a current state involve complicated infectious conditions exhibited by a bacterial infection owing to the difference in the underlying disease, large gaps of sensitivity and the like of the animal spices toward the bacterial strain, and thus, systems of the infection model have been individually established depending on the purpose of the research. Examples of the system of the infection model which are well known at present include: (i) a process in which formation of intraabdominal abscess is allowed through the infection of any of various microorganisms into a peritoneal cavity of a mouse or a rat to chase the pathological state of sepsis (biphasic infection theory), (ii) particularly, in instances to study dynamics of the infection under a lowered immune state, a process in which infection of an attenuated microorganism such as Pseudomonas aeruginosa or the like is rendered through decreasing leukocytes by previously administering cyclophosphamide, and a process in which adhesion of Pseudomonas aeruginosa is allowed through making burn injury in a wide area of the skin with an electric trowel in order to facilitate the infection, (iii) in instances to examine dynamics of a living humoral factor such as cytokine released by a macrophage, neutrophil and the like, a process in which a pathological state of sepsis caused by the administration of an Escherichia coli related bacterium such as Escherichia coli and LPS of the same is observed, (vi) to determine the dynamics of tissue images of MOF caused by peritonitis observed in ICU and digestive surgery through allowing invasion of an enteric bacterium by cecal ligation and puncture (CLP) of a cecum of a rat (subacute superinfectious peritonitis model); and the like.

Requirements for preferred animal model include: (I) possible migration of bacteria from a primary focus of the infection into blood (direct administration of bacteria within blood causes bacterial shock in many cases, which can not be controlled as a pathological state), (II) capability of securing sufficient amount of the blood when comparison and examination is executed with time by both test processes, and of performing collection of blood without causing contamination and secondary infection at the site of blood collection, (III) capability of securing phagocytes in an identical amount to that in human because less phagocytes such as neutrophils may be present depending on the animal spices and age of weeks, (IV) no great influence on each individual and detection sensitivity by alteration of an immune system by the stress and shock upon blood drawing through frequent collection of blood, (v) possible securement of number of experiments to some extent such that individual difference is avoided, and the like.

Biphasic infection theory that is the most general process as a system to produce an animal model is a historically conventional system, which starts in 1931 by Meleney et al, and investigated and established by Hite (1949), Mergehagen (1958) and McDonald (1963) et al. This model has been established on the basis of a theoretical ground of an infection route in which a secondary infection focus is formed from the bacterium of a primary focus via blood irrespective of whether the bacterium is an anaerobe or aerobe. Thus, this model has been generally used as an infection model of sepsis. However, because this system was established as a system for use in analysis of pathologic states of bacterial infectious disease, no importance is attached to the amount of bacteria which migrate from the abdominal cavity into the blood. Therefore, because the amount of bacteria which was intraperitoneally administered is not reflected to the amount of bacteria in the blood due to the influence of the individual difference of each rat, it is difficult to consider the difference resulting from the administered amount of bacteria in an in vivo test.

In addition, Bacterial Translocation methods also involve problems. A factor for impossibility of easy comparison of the detection sensitivity of attenuated infectious bacteria such as Escherichia coli, Enterobactor cloacae, Klebsiella pneumoniae, Enteroccocus faecalis, Staphylococcus epidermidis and the like may involve that these bacterial strains are indigenous bacteria which are enteric and mucosal. In this in vivo test system, analysis of a pathogenic state of sepsis is intended rather than the dynamics of the administered bacteria, therefore, invasion of a bacterial strain other than the administered bacteria is not considered. Recently, also in clinical scene and animal experiments, it has been argued that enteric canal permeability is promoted by peritonitis, and that sepsis is caused through migration of enteric bacteria into the blood. Further, in clinical scene, there exist cases in which the primary focus can not be specified in MOF resulting from peritonitis, and thus attention has been drawn of the relationship with bacterial translocation.

Moreover, in an animal experiment, it was reported that in cases of intraperitoneal administration of Enterococcus faecalis in this in vivo test model, bacterial translocation from the enteric canal was caused due to the inflammation stress by peritonitis as an attraction, and thus, Enterococcus faecalis in the enteric canal was separated at the ratio of 33% (9/27). In addition, Steffen et al. also acknowledged that many enteric bacteria migrate into blood in this in vivo test model.

Further, although not by the bacterial translocation, in peritonitis caused by a cecum ligature puncturing method for use in rat sepsis models, it is also reported that Escherichia coli, Enterobactor cloacae, Klebsiella pneumoniae, Enteroccocus faecalis and Staphylococcus epidermidis were reparated at 12 hours later from the blood.

Because relationships between the causative microorganism of an infectious disease and the host is extremely complicated, it is further difficult to establish an ideal animal model which mimics various human infectious diseases, e.g., sepsis and bacteremia. Thus, experimental models of infectious diseases have been desired which enable the in vitro evaluation of phagocytotic ability and/or germicidal capacity of phagocytes, and which are stable and can be widely applied irrespective of species of the foreign microorganism, while retaining the morphology of the phagocyte, as an aid in diagnoses of infectious diseases including bacteremia and sepsis, and in determination of drug efficacy for developing therapeutic drugs for an infectious disease, however, current status is that those which satisfactorily meet the demand have not been provided.

### DISCLOSURE OF INVENTION

An object of the present invention is to provide an experimental model which permits the evaluation of a phagocytotic function of phagocytes in vitro, taking into account of such a current status. Moreover, another object of the present invention is to provide a process which permits the evaluation of an immune function and efficiency of differentiation to phagocytes through the use of such an experimental model. Furthermore, still another object of the present invention is to provide a process for screening various kinds of drugs such as immune function stimulators, anticancer agents, leukocyte differentiation factors, antibiotics and the like; a process for clinical laboratory test in which dosage regimen of various agents are examined; and the like by using such an experimental model. Additionally, provided is a process in which performance tests such as sensitivity tests, specificity tests, reproducibility tests and the like are conducted, or in which the aforementioned experimental model is used as a positive control, by a kit through: obtaining phagocytes from a clinical specimen containing phagocytes derived from a living body; fixing thus resulting phagocytes; executing a treatment for promoting permeability of the cell membranes; executing a treatment for exposing the DNA of a foreign microorganism predicted as existing in the phagocytes; carrying out in situ hybridization using a DNA probe for detection capable of hybridizing with the DNA under a stringent condition; and detecting and/or detecting the foreign microorganism by the resulting signal.

The present invention was accomplished in light of the current status described hereinabove in detail, and aspects thereof are as described in the following Items 1 to 40.

1. A digested phagocyte prepared by contacting in vitro a phagocyte with a foreign microorganism and isolating the phagocyte so contacted.

2. The digested phagocyte according to Item 1 wherein a turbidity of bacterial liquid (O.D.=600 nm) of the foreign microorganism used for in vitro contact between the phagocyte and the foreign microorganism is 0.01 to 0.03.

3. The digested phagocyte according to Item 1 or 2 wherein a density of the phagocyte digested with the foreign microorganism is 1×10⁴ cells/μl to 5×10⁴ cells/μl.

4. The digested phagocyte according to any one of Items 1-3 wherein said foreign microorganism is a gram negative bacterium.

5. The digested phagocyte according to any one of Items 1-3 wherein said foreign microorganism is one or more microorganism selected from the group consisting of Staphylococcus aureus, Staphylococcus epidermidis, Enterococcus faecalis, Pseudomonas aeruginosa, Escherichia coli and Candida albicans, and a mixture thereof.

6. A process for producing a phagocyte digested with a foreign microorganism comprising the steps of:

contacting in vitro a phagocyte with a foreign microorganism; and

isolating the phagocyte.

7. The process according to Item 6 wherein a turbidity of bacterial liquid (O.D.=600 nm) of the foreign microorganism used for in vitro contact between the phagocyte and the foreign microorganism is 0.01 to 0.03.

8. The process according to Item 6 or 7 wherein a density of the phagocyte digested with the foreign microorganism is 1×10⁴cells/μl to 5×10⁴ cells/μl.

9. The process according to any one of Items 6-8 wherein said foreign microorganism is a gram negative bacterium.

10. The process according to any one of Items 6-8 wherein said foreign microorganism is one or more microorganism selected from the group consisting of Staphylococcus aureus, Staphylococcus epidermidis, Enterococcus faecalis, Pseudomonas aeruginosa, Escherichia coli and Candida albicans, and a mixture thereof.

11. A process for detecting and/or identifying a digested foreign microorganism comprising the steps of:

fixing the phagocyte digested with a foreign microorganism according to any one of Items 1-5;

treating to promote permeability of the cell membrane of the phagocyte;

treating to expose DNA of the foreign microorganism existing in the phagocyte;

in situ hybridizing under a stringent condition between a DNA probe which can detect hybridization and the DNA; and

detecting and/or identifying the digested foreign microorganism by the resulting signal.

12. A process for evaluating a phagocytotic function against a foreign microorganism comprising the steps of:

fixing the phagocyte digested with a foreign microorganism according to any one of Items 1 to 5;

treating to promote permeability of the cell membrane of the phagocyte;

treating to expose DNA of the foreign microorganism existing in the phagocyte;

in situ hybridizing under a stringent condition between a DNA probe which can detect hybridization and the DNA; and

identifying by the resulting signal the phagocytosis and/or killing ability of the phagocyte against the foreign microorganism.

13. The process according to Item 11 or 12 wherein said process includes at least one aspect of:

(1) the density (X cells/ml) of the phagocytes to be fixed is 5×10⁶ cells/ml<X cells/ml<1×10⁸ cells/ml;

(2) in said exposing step of the DNA, lysostafin having the titer of 1 unit/ml to 1,000 unit/ml is used;

(3) in said exposing step of the DNA, lysozyme having the titer of 1,000 unit/ml to 1,000,000 unit/ml is used;

(4) in said exposing step of the DNA, N-acetylmuramidase having the titer of 10 unit/ml to 10,000 unit/ml is used;

(5) in said exposing step of the DNA, zymolase having the titer of 50 unit/ml to 500 unit/ml is used;

(6) in said in situ hybridization step, a surfactant is used;

(7) said DNA probe for detection is one or more DNA probe having the chain length of 350 to 600 base length; and

(8) the concentration of said DNA probe for detection is 0.1 ng/μl to 2.2 ng/μl.

14. The process according to Item 13 wherein one or more enzyme selected from lysostafin, lysozyme, N-acetylmuramidase and zymolase is used in said exposing step of the DNA, with the titer of lysostafin being 10 unit/ml to 100 unit/ml; the titer of lysozyme being 10,000 unit/ml to 100,000 unit/ml; the titer of N-acetylmuramidase being 100 unit/ml to 1,000 unit/ml; and the titer of zymolase being 100 unit/ml to 500 unit/ml.

15. The process according to any one of Items 11 to 14 wherein an enzyme is used in said exposing step of the DNA, and wherein the temperature to allow the reaction of the enzyme is 26° C. to 59° C., with the time period of the reaction of the enzyme being 15 minutes to 120 minutes.

16. The process according to any one of Items 11 to 15 wherein a substance for retaining the morphology of the phagocyte is additionally used in said exposing step of the DNA.

17. The process according to Item 16 wherein said substance is phenylmethylsulfonyl fluoride.

18. The process according to Item 17 wherein the concentration of said phenylmethylsulfonyl fluoride is 10 μmol/l to 10 mmol/l.

19. The process according to any one of Items 16 to 18 wherein said substance is a substance dissolved in dimethylsulfoxide.

20. The process according to Item 19 wherein the concentration of said dimethylsulfoxide is less than 5%.

21. The process according to any one of Items 11 to 20 wherein the DNA and the DNA probe is hybridized in the presence of a surfactant in said in situ hybridization step.

22. The process according to Item 21 wherein said surfactant is an anion surfactant.

23. The process according to Item 22 wherein said anion surfactant is sodium dodecylsulfate.

24. The process according to any one of Items 11 to 23 wherein the temperature to allow the hybridization reaction is 25° C. to 50° C., with the time period of the hybridization reaction being 30 minutes to 900 minutes in said in situ hybridization step.

25. A process for evaluating a phagocytotic function against a foreign microorganism comprising the steps of:

fixing the digested phagocyte according to any one of Items 1 to 5;

staining the phagocyte with a dye; and

identifying the phagocytosis and/or killing ability of the phagocyte against the foreign microorganism by the detection through observation by microscopic examination on cell morphology which is characteristic in cells during or after phagocytosis.

26. A process for evaluating an immune function comprising the steps of:

isolating phagocytes from a subject;

evaluating a function of the phagocytes using the process for evaluating a phagocytotic function according to any one of Items 12 to 25; and

evaluating the immune function of the subject by comparing the evaluation result to that of the function of normal phagocytes.

27. The process according to Item 26 wherein said immune function is a phagocytotic ability of a microorganism by a leukocyte.

28. The process according to item 27 wherein said immune function is a phagocytotic ability against a microorganism by a leukocyte of a patient who received the radiation exposure or the administration of an anticancer agent.

29. A process for evaluating differentiation efficiency into a phagocyte comprising the steps of:

evaluating a phagocytotic function against a foreign microorganism according to any one of Items 12 to 25; and

evaluating the phagocytotic function in a time dependent manner to identify the alteration.

30. A process of the evaluation for determining an effect of a modulator of phagocytotic function comprising the steps of:

allowing phagocytosis by incubating a suspension of a foreign microorganism and phagocytes in the presence and absence of a phagocytotic function modulator; and

comparing the phagocytotic function in the presence and absence of said phagocytotic function modulator using the process for evaluating a phagocytotic function against a foreign microorganism according to any one of Items 12 to 25.

31. A process for screening a modulator of phagocytotic function comprising the steps of:

allowing phagocytosis by incubating a suspension of a foreign microorganism and phagocytes in the presence and absence of a candidate agent supposed to have a modulatory action toward the phagocytotic function; and

comparing the phagocytotic function in the presence and absence of said agent using the process for evaluating a phagocytotic function against a foreign microorganism according to any one of Items 12 to 25.

32. A clinical testing process comprising the steps of:

obtaining phagocytes from a subject prior to and following the administration of an agent to the subject;

evaluating a function of the phagocyte using the process for evaluating a phagocytotic function according to any one of Items 12 to 25; and

examining a dosage regimen of the agent judging from the effect of the agent determined on the basis of the evaluation result.

33. A performance testing process of a kit for evaluating a phagocytotic function which comprises fixing phagocytes, treating to promote permeability of the cell membranes of the phagocytes, treating to expose the DNA of a foreign microorganism in the phagocytes, in situ hybridize under a stringent condition between the DNA and a DNA probe which can detect hybridization; and evaluating the phagocytotic function by the resulting signal, said kit has;

(1) the foreign microorganism,

(2) at least one or more enzyme(s) selected from the group consisting of lysostafin, lysozyme, N-acetylmuramidase and zymolase used in said exposing step of the DNA, and

(3) one or more DNA probe(s) for detection,

said process is characterized in that the digested phagocyte according to any one of Items 1 to 5 is used.

34. A performance testing process of a kit for detecting and/or identifying a foreing microorganism which comprises obtaining phagocytes from a clinical specimen containing phagocytes derived from a living body, fixing the phagocytes so obtained, treating to promote permeability of the cell membranes of the phagocytes, treating to expose the DNA of the foreign microorganism predicted as existing in the phagocytes, in situ hybridizing under a stringent condition between the DNA and a DNA probe which can detect hybridization, and detecting and/or identifying the foreign microorganism by the resulting signal,

the process is characterized in that the digested phagocyte according to any one of Items 1 to 5 is used.

35. The performance testing process according to Item 33 or 34 wherein said performance test is a sensitivity test, a specificity test or a reproducibility test.

36. The performance testing process according to Item 33 or 34 wherein the digested phagocyte according to any one of Items 1 to 5 is used as a positive control.

37. The process according to any one of Items 11 to 36 wherein the process further comprises a step prior to said fixing step to put the digested phagocyte onto a solid support which is a slide glass coated with 3-aminopropyltriethoxysilane.

38. The process according to any one of Items 11 to 37 wherein a dye for clarifying the contrast between the signal and the cell is used upon the detection of said signal.

39. The process according to any one of Items 11 to 38 wherein said phagocyte is from blood.

40. A kit for evaluating a phagocytotic function by fixing the digested phagocytes according to any one of Items 1 to 5, treating to promote permeability of the cell membranes of the phagocytes, treating to expose DNA of the foreign microorganism in the phagocytes, in situ hybridizing under a stringent condition between the DNA and a DNA probe which can detect hybridization; and evaluating the phagocytotic function by the resulting signal, wherein said kit has;

(1) the foreign microorganism,

(2) at least one or more enzyme(s) selected from the group consisting of lysostafin, lysozyme, N-acetylmuramidase and zymolase used in said exposing step of the DNA, and

(3) one or more DNA probe(s) for detection.

### BRIEF DESCRIPTION OF DRAWINGS

FIG. 1 is a view illustrating results of in situ hybridization carried out (a) without the surfactant (SDS) and (b) with the surfactant (SDS).

FIG. 2 is a view illustrating the states obtained upon fixing with various leukocyte cell densities.

FIG. 3 is a view illustrating the activity of lytic enzyme on (a) Staphylococcus aureus and Staphylococcus epidermidis, (b) Pseudomonas aeruginosa and Escherichia coli, and (c) Enterococcus faecalis in a time dependent manner.

FIG. 4 is a view illustrating concentration dependent effects by the addition of DMSO on a lytic activity of (a) 300 unit/ml N-acetylmuramidase, (b) 10,000 unit/ml lysozyme, and (c) 50 unit/ml lysostafin.

FIG. 5 is a drawing illustrating effects of the addition of PMSF used for suppressing the action of protease which effects deterioration of morphology of leukocytes in respect of (a) 0.2 unit/ml protease alone, (b) addition of 1 μmol/ml PMSF, (c) addition of 10 μmol/ml PMSF, (d) addition of 0.1 mmol/ml PMSF, and (e) addition of 1 mmol/ml PMSF.

FIG. 6 is a view illustrating the occurrence of the alteration of morphology of phagocyte upon phagocytosis of bacteria, in the digested sample prepared according to the present invention.

FIG. 7 is a view illustrating the effects of the enzymatic treatment on digested samples, showing states of: (a) digested sample of S. aureus prior to the enzymatic treatment, (b) digested sample of E. faecalis prior to the enzymatic treatment, (c) sample (a) following the enzymatic treatment, and (d) sample (b) following the enzymatic treatment.

FIG. 8 is a diagrammatic view illustrating the slide glass for smear of digested samples used in the study of the optimal concentration of the probe upon in situ hybridization.

FIG. 9 is a diagrammatic view illustrating the slide glass for smear of digested samples used in the study of the optimal temperature upon in situ hybridization.

FIG. 10 is a view of Southern blotting (upper panel) and electrophoresis (lower panel) illustrating the chain length of the probe for detection obtained by digoxigenin labelling of (a) SA probe and (b) PA probe, and signal intensities by labelling.

FIG. 11 is a view illustrating results of signal detection observed when (a) EC-24, (b) EC-34, (c) EC-39, and (d) mixed probe of probes (a) to (c) as the probe for detection upon in situ hybridization for E. coli digested sample.

FIG. 12 is a diagrammatic view illustrating a slide glass for smear of digested samples.

FIG. 13 is a view illustrating results of signal detection observed when in situ hybridization was carried out using corresponding probe for the detection to each of digested samples of (a) SA, (b) SE, (c) PA, (d) EF and (e) EK.

FIG. 14 is a view illustrating states in which the probe for detecting SA specifically presents signals for the SA digested sample.

### BEST MODE FOR CARRYING OUT INVENTION

According to one embodiment of the present invention, an experimental model (hereinafter, referred to as digested sample ) is provided characterized in that: phagocytes are brought into contact in vitro with a foreign microorganism prepared such that turbidity of the bacterial liquid (O.D.=600 nm) is preferably about 0.01 to about 0.03; phagocytes post phagocytosis of a foreign microorganism are prepared by isolating the cells; and adjusting thus resulting phagocytes post phagocytosis of a foreign microorganism to give about 1×10⁴ cells/μl to about 5×10⁴ cells/μl.

The term phagocyte post phagocytosis of a foreign microorganism used herein means a cell attached to a foreign microorganism or a cell including a foreign microorganism involving not alone cells after completing phagocytosis of a foreign microorganism already, but cells during phagocytosis and cells which are ready to the initiate phagocytosis after the adhesion of a foreign microorganism on the cell surface.

The phagocyte referred to herein is not particularly limited as long as it is a cell capable of incorporating a foreign substance as well as a foreign microorganism within the cell of its own, and examples thereof include macrophage, monocyte, neutrophil, eosinophil and the like. In addition, a phagocyte system such as U937 cells, HL60 cells or the like may be also used.

Because phagocytes derived from a living body are also included in body fluids such as e.g., blood, tissue fluids, lymph fluid, cerebrospinal fluid, pyo, mucus, snot, sputum, urine, ascites and the like, or dialysis drainage, or otherwise lavage obtained after washing nasal cavity, bronchus, skin, any of various organs, bone or the like, phagocytes can be also prepared from these sources. In addition, phagocytes can be prepared also from a tissue such as skin, lung, kidney, mucosa or the like. Macrophage which is one of phagocytes transforms into a variety of morphology such as monocyte, pulmonary alveolus macrophage, peritoneal cavity macrophage, fixed macrophage, free macrophage, Hansemann macrophage, inflammatory macrophage, hepatic Kupffer cell, cerebral microglia cell and the like, therefore, any tissue including these may be used also as a source of the phagocyte in addition to blood. For preparing phagocytes from a tissue, for example, cells are detached by using an enzyme such as trypsin after collecting the tissue to isolate phagocytes which are present in the tissue.

In order to obtain a phagocyte (leukocyte) fraction from a body fluid or the like, any known method can be used. For example, about 5 ml of heparinized venous blood (10 ml when number of leukocytes is small) is collected, followed by mixing of this blood with a reagent for separating blood (225 mg of sodium chloride, 1.5 g of dextran (MW: 200,000-300,000), adjusted to give the total volume of 25 ml with sterile purified water) at a ratio of 4:1 and leaving to stand still at about 10° C. to about 40° C. for about 15 minutes to about 120 minutes, preferably at 37° C. for about 30 minutes. Accordingly, a leukocyte fraction (upper layer) can be obtained.

Leukocytes can be obtained by centrifugation of the resultant leukocyte fraction at 0° C. to about 20° C. for about 3 minutes to about 60 minutes at about 100 to about 500×g, preferably, at 4° C. for 10 minutes at about 140 to about 180×g. When erythrocytes are contaminated upon this operation, it is preferred that a hemolysis operation is conducted. For example, 1 ml of sterile purified water may be added to a pellet of leukocytes and suspended, and immediately thereafter an excess amount of PBS (18.24 g of sodium chloride, 6.012 g of sodium monohydrogen phosphate 12 hydrate, 1.123 g of sodium dihydrogen phosphate dihydrate, adjusted to give the total volume of 120 ml with sterile purified water (PBS stock solution; hereinafter referred to simply as “PBS stock solution ) diluted to 20 fold with sterile purified water; hereinafter referred to simply as “PBS) may be added thereto to result in isotonization, followed by centrifugation once again at 40° C. for. 10 minutes at about 140 to about 180×g.

The foreign microorganism which may result in an infectious disease is not particularly limited as long as it is a microorganism which is subjected to phagocytosis by a phagocyte, and examples thereof include bacteria, fungi, viruses, protozoan, parasites and the like. Examples of bacteria include e.g., staphylococci, Pseudomonas aeruginosa, enterococci, coli bacteria, Streptococci, pneumococci, tubercle bacilli, helicobacter pylori, listeria, yersinia, brucella and the like. Examples of fungi include e.g., candida, aspergillus, actinomyces, coccidioides, blastomyces and the like. Examples of viruses include e.g., influenza virus, polio virus, herpes virus, hepatitis virus, AIDS virus and the like. Examples of protozoan include e.g., amebic dysentery, Trichomonas vaginalis, malaria, toxoplasma and the like. Examples of parasite include trypanosome and the like. In particular, examples of causative microorganism of sepsis or bacteremia include e.g., staphylococci (Staphylococcus aureus, Staphylococcus epidermidis), enterococci (Enterococcus faecalis, Enterococcus faecium, Streptococcus pneumoniae, Streptococcus pyogenes, Streptociccus agalactiae) which are Gram positive bacteria; coli bacteria (Escherichia coli), enterobacter (Enterobacter cloacae), Escherichla coli analogous enteric bacteria (Klebsiella oxytoca, Serratia marcesens, Proteus vulgaris, Citrobacter freundii) such as klebsiella (Klebsiella pneumoniae) which are Gram negative bacteria; pseudomonas (Pseudomonas aeruginosa) which are an aerobic bacilli; clostridium (Clostridium perfringens), bacteroides (Bacteroides fragilis) which are anaerobe and the like. On rare occasions, Acinetobacter calcoaceticus, Aeromonas hydrophilia, Flavobacterium meningosepticum, Bacillus cereus or the like may serve as the cause. Among these, in particular, Gram negative bacteria, or one or more microorganism selected from the group consisting of Staphylococcus aureus, Staphylococcus epidermidis, Enterococcus faecalis, Pseudomonas aeruginosa, Escherichia coli and Candida albicans, and mixtures thereof are suitably used.

For allowing phagocytosis of a foreign microorganism by phagocytes, the foreign microorganism is precultured to give a certain amount previously. After suspending in PBS thus collected microorganisms following proliferation, they are diluted in PBS to adjust the turbidity of the bacterial liquid (O.D.=600 nm) to about 0.001 to about 0.1, preferably about 0.01 to about 0.03 measured by an absorption meter. Thus produced bacterial liquid is transferred to separate flasks for culture, and left to stand still at room temperature for about: 30 minutes. Heparinized healthy human blood is collected, and the aforementioned reagent for separating hemocyte is added thereto at a ratio of approximately 4:1, followed by leaving to stand still at about 20° C. to about 40° C., preferably at 37° C. for about 30 minutes to yield a leukocyte fraction. Thus obtained leukocyte fraction is suspended in PBS. The supernatant in the flask for culture which had been charged with the foreign microorganism is gently discarded, and the leukocyte fraction diluted in PBS is added to the flask followed by leaving to stand still at room temperature for about 10 minutes. The supernatant in the flask for culture is discarded, and leukocytes adhered on the bottom of the flask are recovered in a centrifuge tube using PBS containing 0.02% EDTA, and are collected by e.g., centrifugation at 4° C. for 10 minutes at about 140 to about 180×g. When contamination of erythrocytes is found in thus collected leukocytes, leukocytes may be collected by: allowing hemolysis through gently suspending the precipitates of leukocytes in sterile purified water, isotonization through adding PBS, followed by centrifugation once again at 4° C. for 10 minutes at about 140 to about 180 g. The collected leukocytes are suspended in PBS, and cell number is counted with a counting chamber to adjust to give about 1×10⁴ cells/μl to about 5×10⁴ cells/μl.

Process for fixing leukocytes may involve for example, carrying out Carnoy fixation.

Specifically, leukocytes are supported on a carrier capable of supporting leukocytes (supporting carrier), immersed in Carnoy's fixative(a mixed solution at a volume ratio of ethanol : chloroform : acetic acid=6:3:1) for about 20 minutes, thereafter immersed in about 50% to about 90%, preferably about 75% ethanol solution for about 5 minutes, and then completely air dried.

The supporting carrier described above is preferably any of those made from an insoluble material, and for example, glass, metal, synthetic resins (polystyrene, polyethylene, polypropylene, polyvinyl chloride, polyester, polyacrylic ester, nylon, polyacetal, fluorine resin and the like), polysaccharides (cellulose, agarose and the like) are preferred.

The insoluble supporting carrier may be in any of various forms such as, for example, plate-like, tray-like, spherical, fibrous, cylindrical, discal, vessel-like, cell-like, tubular and the like. In particular, preferable supporting carrier for use in one embodiment of the present invention is a slide glass. Examples of the slide glass include e.g., a slide glass (item number: MS311BL) manufactured by JAPAN AR BROWN CO., LTD. This slide glass (item number: MS311BL) is provided with 14 circular wells having the diameter of 5 mm addition, upon practical use, it is preferred that an APS coated slide glass is used which is a slide glass with 3-aminopropyltriethoxysilane (APS, SIGMA) coated thereon for the purpose of improving adhesiveness of cells. Alternatively, a slide glass with poly-L-lysine or gelatin coated thereon may be also used.

For producing an APS coated slide glass, a slide glass (item number: MS311BL) is first fixed on a slide holder, and thereafter is washed by immersing in a diluted neutral detergent for 30 minutes, and the detergent is sufficiently removed with running water. Next, the slide glass is washed with purified water and dried sufficiently at high temperature (100° C. or greater) followed by leaving to stand to cool at room temperature. Then, the slide glass is immersed in acetone containing 2% APS for 1 minute, and immediately thereafter washed briefly with acetone and sterile purified water sequentially followed by air drying. Further, after conducting the operation of immersing the slide glass in acetone containing about 1 to about 10% APS for 1 minute, followed by immediate and brief washes with acetone and sterile purified water in a sequential manner and air drying once again, the APS coated slide glass can be produced by drying at about 20° C. to about 60° C., preferably at 42° C.

When the leukocytes are supported on the APS coated slide glass, it is preferred that leukocytes are smeared on each well such that they are spread over to give a single layer and air dried. It is preferred that phagocytes for use in fixing are prepared such that the density (x cells/ml) is about 5×10⁶ cells/ml<x cells/ml<about 1×10⁸ cells/ml; and preferably about 1×10⁷ cells/ml x cells/ml about 5×10⁷ cells/ml.

Moreover, corresponding to such alteration of density of the phagocytes per 1 ml, cell number of the leukocytes fixed on the APS coated slide glass per 1 well (y cells/well (diameter: 5 mm) is preferably adjusted to be about 2.5×10⁴ cells/well<y cells/well<about 5×10⁵ cells/well, and preferably about 5×10⁴ cells/well y cells/well about 2.5×10⁵ cells/well. Specifically, a small amount of PBS is added to a leukocyte pellet obtained by centrifugation of the leukocyte fraction at 4° C. for about 10 minutes at about 140×g to about 180×g followed by suspension, and the cell number of the leukocytes is counted using a counting chamber. Preparation can be perfected by smearing 5 μl of the leukocyte suspension, which was prepared with PBS such that cell number becomes about 5×10⁴ cells/well to about 2.5×10⁵ cells/well, on each well of the APS coated slide glass to allow the leukocytes spread to form a single layer followed by complete air drying.

As a treatment for promoting permeability of the membranes of phagocytes, a process may be employed in which immersion is conducted in PBS for about 3 to about 30 minutes, followed by immersing in a solution of an enzyme pretreatment reagent (prepared by mixing 1.25 g of saponin, 1.25 ml of t-octylphenoxypolyethoxyethanol (specific gravity: 1.068 to 1.075 (20/4° C.), pH (5 w/v %) 5.5-7.5) and 25 ml of a PBS stock solution, and adjusting to give the total volume of 50 ml with sterile purified water) diluted to about 2 to about 50 fold in sterile purified water, and allowing infiltration on a shaker for about 3 to about 30 minutes.

In a treatment for exposing the DNA of the causative microorganism of an infectious disease in the phagocytes, an enzyme reagent solution is prepared by adding 1 ml of an enzyme reagent dissolving solution (prepared by about 100 fold dilution of dimethylsulfoxide (DMSO) which contains phenylmethyl-sulfonylfluoride (PMSF) in PBS) to an enzyme reagent (N-acetylmyramidase, lysozyme and/or lysostafin) per 1 slide, and thereafter, 1 ml of this enzyme solution is dropped on a site of the leukocyte smear, and left to stand still for about 10 to about 60 minutes in a humid box at about 20° C. to about 60° C., preferably at about 37° C. to about 42° C. Then, it is immersed in PBS containing 0.2 mol/l hydrochloric acid (prepared by adding hydrochloric acid to the PBS stock solution, 20 fold dilution in sterile purified water, and adjusting to give the final concentration of hydrochloric acid of 0.2 mol/l) and thus the object is achieved by allowing infiltration on a shaker for 3 to 30 minutes as it is. Since DMSO has the potential of lowering the activity of lysozyme and lysostafin at the concentration of 5% or greater, it is preferably used at the concentration of less than 5%. Except for PMSF as a substance for retaining the morphology of the phagocytes, other known protease inhibitor, e.g., tosyl lysine chloromethyl ketone (TLCK) and a mixture thereof may be also used. In such a case, a solvent such as DMSO may be changed ad libitum.

In regard of preferable range of the titer of each enzyme used as an enzyme reagent, although lysostafin exerts a sufficient effect at a titer of 1 unit/ml upon lysis of Staphylococcus aureus, lysostafin having the titer of 10 unit/ml or greater was required for lysis of Staphylococcus epidermidis. Therefore, optimal titer of lysostafin may be set to 1 unit/ml to about 1,000 unit/ml, and preferably about 10 unit/ml to about 100 unit/ml. Further, upon lysis of Enterococcus faecalis, lysis did not occur when the titer of N-acetylmuramidase is about 10 unit/ml or less, while the titer of lysozyme was fixed to be about 10,000 unit/ml. In respect of lysozyme, when the titer of N-acetylmuramidase was fixed to be 100 unit/ml, lysis did not occur with the titer of lysozyme of 1,000 unit/ml or less. Therefore, optimal titer of N-acetylmuramidase may be set to be about 10 unit/ml to about 10,000 unit/ml, and preferably about 100 unit/ml to about 1,000 unit/ml, whilst the optimal titer of lysozyme may be set to be about 1,000 unit/ml to about 1,000,000 unit/ml, and preferably about 10,000 unit/ml to about 100,000 unit/ml. Furthermore, in instances where the causative microorganism is a fungus such as Candida albicans, the range of titer may be about 50 unit/ml to about 500 unit/ml, preferably about 100 unit/ml to about 500 unit/ml of zymolase. Additionally, when zymolase is used in particular, it is preferred that PMSF or known protease inhibitor is used.

Moreover, depending on the difference of components in Gram positive bacteria and Gram negative bacteria, in other words, on the difference in peptidoglycan or lipopolysaccharide, the enzyme to be used may be optionally selected. Particularly, irrespective of whether Gram positive bacterium or Gram negative bacterium is, two or more enzymes may be used in combination for the purpose of achieving lysis more effectively. According to the present invention, it was revealed that by using a mixture of three kinds, lysozyme, lysostafin and N-acetylmuramidase, lytic activity was elevated in comparison with the case where a single enzyme was used.

Temperature of the enzymatic treatment may be preferably about 4° C. to about 60° C. for Staphylococcus aureus; higher than about 25° C., preferably about 37° C. or higher for Staphylococcus epidermidis; and higher than about 25° C. and less than about 60° C., preferably about 37° C. to about 42° C. for Enterococcus faecalis. Accordingly, it is most preferred that optimal temperature for the enzymatic treatment is set to be about 37° C. to about 42° C. Additionally, critical temperature is expected to be about 26° C. to about 59° C. in the common range for those three kinds of the bacteria.

Further, time period of the enzymatic treatment may be 20 minutes or longer for any of digested samples of Staphylococcus aureus, Staphylococcus epidermidis and Enterococcus faecalis (inadequate in 0 minute and 10 minutes), and because no bacterial body was found within the leukocytes, the time period is preferably at least about 15 minutes or longer, preferably about 20 minutes or longer, and in addition, optimal time period of the enzymatic treatment shall be about 30 minutes to about 60 minutes. Moreover, the time period of the enzymatic treatment may be about 15 minutes to about 120 minutes.

N-acetylmuramidase is an enzyme which lowers the absorbance at 600 nm when thermally treated dry powder of Enterococcus faecalis by a heat treatment and N-acetylmuramidase are subjected to a reaction in a 5 mmol/l Tris-HCl buffer (pH 6.0) containing 2 mmol/l magnesium chloride at 37° C. for 5 minutes. Additionally, when the enzymatic activity at 37° C., pH 7.0 in 1 minute to lyse 1 ug of cells of Streptococcus salivarius (IFO 3350), which was subjected to a heat treatment, is determined as 1 unit, it is preferred that one having the enzymatic activity of 2,000 unit/mg or greater is used.

Lysozyme is an enzyme which lowers the absorbance at 600 nm when Micrococcus luteus and lysozyme were subjected to a reaction in PBS at 37° C. for 5 minutes. Furthermore, when the enzymatic activity to lower the absorbance at 540 nm of Micrococcus luteus by 0.001 at 35° C., pH 6.2 in 1 minute is determined as 1 unit, it is preferred that one having the enzymatic activity of 50,000 unit/mg or greater is used.

Lysostafin is an enzyme which lowers the absorbance at 600 nm when Staphylococcus epidermidis and lysostafin are subjected to a reaction in PBS at 37° C. for 5 minutes. Furthermore, when the enzymatic activity to lower the absorbance at 620 nm of Staphylococcus aureus from 0.240 to 0.125 at 37° C., pH 7.5 in 10 minutes is determined as 1 unit, it is preferred that one having the enzymatic activity of 500 unit/mg or greater is used.

Zymolase (trade name: Zymolyase, Seikagaku Corporation) is an enzyme prepared from a culture liquid of Arthrobacter lutesul, having a potent lytic activity against cell walls of yeast living cells. Essential enzyme involving in lysis of cell walls included in zymolase is -1,3-glucan lanimaripentaohydrolase, which acts on a glucose polymer having -1,3-bonds to produce lanimaripentaose as a major product. Zymolyase-100T, which is purified by ammonium sulfate fractionation, and further purified by affinity chromatography (Kitamura, K. et al.; J. Ferment. Technol., 60, 257, 1982), has the activity of 100,000 unit/g. However, the activity of this enzyme is known to be altered depending on the type of yeast to be a substrate, culture condition and growing stage (Kitamura, K. et al.; J. Gen. Appl. Microbiol., 20, 323, 1974, Kitamura, K. et al.; Agric. Biol. Chem., 45, 1761, 1981, Kitamura, K. et al.; Agric. Biol. Chem., 46, 553, 1982). Zymolyase-100T includes about 1.0×10⁷ unit/g of -1,3-glucanase, about 1.7×10⁴ unit/g of protease, and about 6.0×10⁴ unit/g mannase, however, DNase and RNase are not found therein (Kitamura, K. et al.; J. Gen. Appl. Microbiol., 18, 57, 1972). In addition, the optimal pH of Zymolyase is about 5.5 to about 8.5, and preferably about 6.5 to about 7.5, whilst the optimal temperature is about 25° C. to about 55° C., and preferably about 35° C. to about 45° C. Moreover, lysis spectrum (genus name) against yeast (cells in logarithmic growth phase) includes Ashbya, Candida, Debaryomyces, Eremothecium, Endomyces, Hansenula, Hanseniaspora, Kloekera, Kluyveromyces, Lipomyces, Helschkowia, Pichia, Pullularia, Torulopsis, Saccharomyces, Saccharomycopsis, Saccharomycodes, Schwanniomyces and the like.

In particular, examples of those in genus candida include Candida albicans, Candida tropicalis, Candida parasilosis, Candida galacta, Candida guilliermondii, Candida krusei, Cryptococcus neoformans and the like. As an activator of this enzyme, an SH compound, e.g., cysteine, 2-mercaptoethanol, dithiothreitol and the like can be used.

Fungi belonging to these genera may be also used in the present invention. According to this enzyme, an enzymatic activity required for decreasing about 30% of A₈₀₀ of a reaction liquid (enzyme: 1 ml of a 0.05 to 0.1 mg/ml solution, substrate: 3 ml of a beer yeast suspension (2 mg dry weight/ml), buffer: 5 ml of M/15 phosphate buffer (pH 7.5), adjusted to give the total volume of 10 ml with 1 ml of sterile purified water) using the beer yeast suspension as a substrate at about 25° C. within two hours is determined as 1 unit. Zymolyase-100T has the activity of 100,000 unit/g.

It is preferred that the concentration of PMSF (added in order to protect the leukocytes from protease so that the morphology thereof is retained) which is used as a solvent for the enzyme reagent is in the range of 10 μmol/l to 10 mmol/l, and preferably 0.1 mmol/l to 1 mmol/l, because effects were observed at the concentration of 10 μmol/l or greater, while deterioration of morphology of leukocytes was completely suppressed at the concentration of 0.1 mmol/l or greater. In addition, it is preferred that the concentration of DMSO is less than 5%, preferably 2% or less, and further approximately the concentration of 1%. As a consequence, the enzyme reagent dissolving solution is preferably prepared by 100 to 1,000 fold dilution of dimethylsulfoxide (DMSO) which contains 0.1 mol/l phenylmethylsulfonylfluoride (PMSF) in PBS.

Following the step of exposing the DNA of the causative microorganism of an infectious disease, the step of acetylation of cell membrane proteins may be inserted. Specifically, it can be carried out through immersing the slide glass in an acetylation reagent, which was prepared by adding acetic anhydride to an acetylating reagent (7.46 g of triethanolamine, an appropriate amount of hydrochloric acid, adjusted to give the total volume of 50 ml with an appropriate amount of sterile purified water) and diluting about 2 fold to about 50 fold, preferably about 10 fold in sterile purified water to give the final concentration of acetic anhydride of 0.1 to 3.0%, preferably 0.8%, followed by shaking for 5 to 30 minutes on a shaker. Thereafter, the slide glass is sequentially immersed in 75%, 85%, and 98% ethanol for 2 to 5 minutes respectively, and completely air dried.

Additionally, following the step of acetylation of cell membrane proteins, the step of forming a single stranded DNA by an alkali treatment of the DNA of the causative microorganism of an infectious disease can be also inserted. Specifically, it can be carried out through immersing the slide glass in PBS which contains about 10 mmol/l to about 300 mmol/l, preferably about 70 mmol/l sodium hydroxide (prepared by adding sodium hydroxide in the PBS stock solution, diluting to 20 fold with sterile purified water to give the final concentration of sodium hydroxide of 70 mmol/l) for about 2 to about 5 minutes. Thereafter, the slide glass is sequentially immersed in 75%, 85%, and 98% ethanol for 2 to 5 minutes respectively, and completely air dried.

Upon carrying out in situ hybridization using a DNA probe for detection capable of hybridizing with the exposed DNA of the causative microorganism of an infectious disease under a stringent condition, for example, a liquid containing the DNA probe for detection prepared in a probe dilution solution (probe solution) is coated on the smeared site, and is left to stand still in a humid box at about 25° C. to about 50° C., preferably at about 37° C. to about 42° C. for about 1 to about 3 hours, preferably for about 2 hours.

Thereafter, a hybridization washing solution (prepared by mixing a hybridization stock solution (13.15 g of sodium chloride, 6.615 g of trisodium citrate dihydrate, adjusted to give the total volume of 75 ml with sterile purified water: hereinafter, referred to as merely hybridization stock solution ) in a ratio of the hybridization stock solution:sterile purified water:formamide=5:45:50) is provided in three staining bottles, and sequentially, the sample is immersed at about 35 to about 45° C., preferably at about 42° C. for 10 minutes, respectively. Then, the sample is immersed in PBS, and shaken as it is on a shaker for about 5 to about 30 minutes. In detail, the probe dilution solution includes 600 μl of salmon sperm DNA, 50 μl of 100× Denhardt's solution 500 μl of hybridization stock solution, 2,250 μl of formamide, 1,000 μl of 50% dextran sulfate. The probe solution preferably includes 15 ng of each DNA probe for detection, which may be adjusted to give the total volume of 50 μl with the probe dilution solution.

Concentration of the probe for SA, SE, PA, EF and EK may be about 0.6 ng/μl to about 1.8 ng/μl, preferably about 0.6 ng/μl to about 1.2 ng/μl. Further, the result of inadequate was brought at 0.06 ng/μl, and the result of adequate was brought at 0.6 ng/μl, therefore, it is preferred that the concentration is set to be at least 0.1 ng/μl or greater. Moreover, because the result of inadequate was brought at 2.4 ng/μl, and the result of adequate was brought at 1.8 ng/μl, it is preferred that the concentration is set to be 2.2 ng/μl or less. In addition, the optimal concentrations of positive control and negative control may be 0.4 to 2.0 ng/μl and 0.6 to 2.0 ng/μl respectively, and preferably 0.6 to 1.0 ng/μl in common.

Further, it is preferred that time period of the hybridization is at least 30 minutes or longer, preferably 60 minutes or longer, and more preferably 90 minutes or longer. More preferred optimal time period of the hybridization may be set to be about 120 minutes to about 900 minutes.

Moreover, to use a surfactant such as sodium dodecyl sulfate (SDS) in the step of in situ hybridization is preferred in light of the capability to improve the detection sensitivity. It is preferred that concentration of SDS is 1% or less, more preferably about 0.1% to about 0.5%, still more preferably about 0.25%. SDS may be added to a solution used upon the hybridization, or may be in the probe dilution solution or in the probe solution, which was mixed beforehand.

Additionally, it is preferred that one or more DNA probe having the chain length of about 350 to about 600 base length, preferably about 350 to about 550 base length is employed as the DNA probe for detection, because the probe is efficiently introduced into phagocytes, and firm contact with the gene of the incorporated foreign microorganism is permitted. It is not intended that base length (number of base pairs) of the subject probe must necessarily fall within the aforementioned range of the base length, but that it is allowable as long as the base length in the aforementioned range is included in the distribution of the base length of the probe. These probes may be used alone, or several kinds of probes (more than one) may be also used. More than one probes may be multiple kinds of probes which can hybridize to one bacterial strain. Alternatively, the kind of the probe may be multiple owing to the presence of multiple types of the bacterial strains although a single probe may be employed for a single bacterial strain. Thus, there is no particulate limitation as far as the kind of the probe is one or more.

These probes preferably comprise a DNA fragment having a sequence which does not any how hybridize with the phagocyte itself, and additionally, they should not cross hybridize with a gene derived from any other strain of microorganism. For example, when a subtraction method is used, a specific probe can be produced in a short period of time. These probes may be prepared and labelled according to a common nick translation process using a non-radioisotopic labelling substance such as fluorescein isothiocyanate (FITC), biotin, digoxigenin (digoxigenin (DIG)-11-dUTP) or the like. Chain length of the probe can be controlled such that most efficient labelling is enabled, by changing the ratio of amount of DNase I and DNA polymerase I added in the nick translation reaction. For example, for efficiently labelling 2 μg of the DNA probe (SA-24), and for regulating the chain length of a probe to enable efficient in situ hybridization with the DNA of a foreign microorganism (base length of about 350 to about 600), when 2 μl of 10 U/μl DNA polymerase I is included in the reaction liquid of total volume of 100 μl, 6 μl of DNase I may be included which was prepared such that about 10 to about 350 mU, preferably about 25 to about 200 mU, more preferably about 50 to about 150 mU is present in total volume of 100 μl. Volume of each enzyme and total volume of the reaction liquid and the like in this instance may optionally vary as long as the proportion according to the aforementioned essential condition for an optimal reaction is kept constant. Further, in other words, when 20U of DNA polymerase I is included in total volume of 100 μl, DNase I may be prepared in an amount of about 10 to about 350 mU, preferably about 25 to about 200 mU, and more preferably about 50 to about 150 mU. In additional other words, when 1 U of DNA polymerase is included, nick translation may be conducted using about 0.5/1,000 to about 17.5/1,000, preferably about 1.25/1,000 to about 10/1,000, and more preferably about 2.5/1,000 to about 7.5/1,000 unit of DNase I. In addition, with DNA in an amount of 1 μg, it is desirable to prepare such that DNA polymerase I is present in an amount of about 10 U., while DNase I is present in an amount of about 5 to about 175 mU, preferably about 12.5 to about 100 mU, and more preferably about 25 to about 75 mU. In respect of other probe, the amount of DNA as well as optimal conditions for the reaction of DNA polymerase I and DNase I can be determined with reference to the optimal conditions for the reaction as described above, and chain length of the probe (base length of about 350 to about 600) can be regulated to result in efficient labelling and efficient in situ hybridization with a foreign microorganism DNA.

The stringent condition for carrying out in situ hybridization may be for example, a condition which comprises incubating in the presence of about 30% to about 60%, preferably about. 50% of formamide, at about 30 to about 50° C., preferably at about 38 to about 42° C. followed by washing.

After carrying out in situ hybridization, an operation of blocking may be performed. Specifically, 1 ml of a blocking reagent (2 ml of normal rabbit serum, 0.5 ml of the PBS stock solution, adjusted to give the total amount of 10 ml with sterile purified water) is dropped on the smear site per one slide glass in a humid box, and left to stand still for 15 to 60 minutes. Thereafter, the blocking reagent is removed.

For detecting a signal which results from the hybridization with a gene derived from the microorganism (genomic DNA or RNA), color reaction may be conducted in which any conventional method for an antigen-antibody reaction is utilized. In other words, after enough washes of the sample following completing the hybridization, an operation for blocking is conducted. Thereafter, a treatment is executed using a conjugate of an anti-FITC antibody, anti-digoxigenin antibody or the like, e.g., an alkaline phosphatase conjugate, and then, color development of the signal is allowed by a color development system of the conjugate to determine the states of hybridization. For example, in instances where the probe labelled with digoxigenin-11-dUTP as described above is used as a probe, an anti-digoxigenin-alkaline phosphatase conjugate is used, and the detection may be conducted through utilizing a substrate which is generally used for alkaline phosphatase (nitroblue tetrazolium, 5-bromo-4-chloro-3-indolyl phosphate and the like). Next, the smear preparation washed after the color reaction is subjected to counter staining with naphthol black Fast Green (20 mg/50 ml, manufactured by Wako Chemical Co.) or the like to observe intracellular signals with a light microscope.

In detail, in order to obtain s signal by hybridization, for example, when a digoxigenin labelled DNA probe is used as a DNA probe for detection, a labelled antibody solution is prepared by diluting a labelled antibody (1.05 unit of alkaline phosphatase labelled anti-digoxigenin antibody solution, adjusted with 12.6 μl of buffer A (746 mg of triethanolamine, 17.5 mg of sodium chloride, 20.3 mg of magnesium chloride hexahydrate, 1.36 mg of zinc chloride, 1,000 mg of bovine serum albumine, an appropriate amount of hydrochloric acid, adjusted to give the total volume of 100 ml with sterile purified water) to give the total volume of 14 μl) in a labelled antibody diluent (8.48 mg of Tris-(hydroxymethyl)-aminomethane, 6.14 mg of sodium chloride, an appropriate amount of hydrochloric acid, adjusted to give the total volume of 0.7 ml with sterile purified water) to 10 to 200 fold, preferably 50 fold, and each 10 μl of this labelled antibody solution is dropped on the smear site, followed by leaving to stand still for 15 to 60 minutes. Thereafter, it is immersed in a solution of a labelled antibody washing solution (1 ml of polysorbate 20, 50 ml of the PBS stock solution, adjusted to give the total volume of 100 ml with sterile purified water) diluted to 2 to 50 fold, preferably 10 fold, and is allowed for infiltration on a shaker for about 5 to about 30 minutes as it is. After repeating this operation twice, it may be immersed in a coloring pretreatment liquid obtained by mixing a coloring pretreatment liquid 1 (6.06 g of Tris-(hydroxymethyl)-aminomethane, 2.92 g of sodium chloride, an appropriate amount of hydrochloric acid, adjusted to give the total volume of 50 ml with sterile purified water) and a coloring pretreatment liquid 2 (5.08 g of magnesium chloride hexahydrate, adjusted to give the total volume of 50 ml with sterile purified water) in an equivalent volume and diluting to approximately 5 fold in sterile purified water, and then shaken for 5 to 30 minutes on a shaker as it is. Thereafter, 1 ml of a coloring reagent (nitroblue tetrazolium (NBT)/5-bromo-4-chloro-3-indolylphosphate (BCIP)) per one slide glass is dropped on the smear site of the slide glass while filtration using a disposable syringe equipped with a 0.2 μm syringe top filter, and is left to stand still under light shielding in a humid box at about 10° C. to about 45° C., preferably at about 37° C. for about 15 to about 60 minutes. Thereafter, it is immersed in a solution of a coloring reagent washing solution (606 mg of Tris-(hydroxymethyl)-aminomethane, 186 mg of ethylenediamine tetraacetate disodium dehydrate, an appropriate amount of hydrochloric acid, adjusted to give the total volume of 50 ml with an appropriate amount of sterile purified water) diluted to about 2 to about 50 fold, preferably about 10 fold for about 2 to about 10 minutes, and is air dried. Then, it is immersed in a solution of a counter staining solution (50 mg of fast green FCF (edible dye, green color No. 3), adjusted to give the total volume of 50 ml with an appropriate amount of sterile purified water) diluted to 2 to 50 fold, preferably to 10 fold and then an acetic acid solution of about 0.1 to about 5%, preferably about 1%. Thereafter, the excess counter staining solution may be washed away by immersing again in a solution of the coloring reagent washing solution described above diluted to about 2 to about 50 fold, preferably about 10 fold, and may be completely air dried. Additionally, the coloring reagent described above may be one prepared separately.

The alkaline phosphatase labelled anti-digoxigenin antibody solution which may be preferably used is one which results in color development in a site of DNA blotting when 1 ng of a digoxigenin labelled DNA is blotted on a membrane for blotting, subjected to blotting, treated with the alkaline phosphatase labelled anti-digoxigenin antibody solution diluted to 10,000 fold, and allowed to react with a coloring substrate (NBT/BCIP), but one which does not result in color development even though similar operation is conducted with a DNA without digoxigenin labelling. Further, the anti-digoxigenin antibody is preferably derived from sheep. In detail, it may be purified from serum of an immunized sheep by an ion exchanging chromatography and an antibody column chromatography.

The coloring reagent (NBT/BCIP solution, pH 9.0 to 10.0) preferably contains 3.3 mg of nitroblue tetrazolium (NBT), 1.65 mg of 5-bromo-4-chloro-3-indolylphosphate (BCIP), 99 μg of N,N-dimethylformamide, 121 mg of Tris-(hydroxymethyl)-aminomethane, an appropriate amount of hydrochloric acid, 58.4 mg of sodium chloride, 101.6 mg of magnesium chloride hexahydrate, and is adjusted to give the total amount of 10 ml with an appropriate amount of sterile purified water. The coloring reagent which may be preferably used is one which exhibits a dark purple signal on the blotted site when a protein labeled with alkaline phosphatase is blotted on a membrane for blotting followed by a treatment of the membrane with the coloring reagent at room temperature under light shielding.

Upon counter staining as described above, an edible dye e.g., yellow No. 4 (tartrazine) can be used for the purpose of further clarification of the contrast between the signal and the cell. The grounds therefor may be difficulties in the counter staining on behalf of the similar color among the purple color developed by the substrate and the blue color developed by naphthol black. When this process was applied to the present invention, it was revealed that the process is beneficial upon the counter staining. The procedure involving in use of an edible dye has not been proposed heretofore.

The process which may be employed for labelling digoxigenin can be a nick translation method. In addition, a PCR method, a random primer labelling method, an in vitro transcription labelling method, a terminal transferase labelling method or the like can be employed.

Determination may be carried out by microscopic examination with a light microscope (×1,000), and observation of at least one color development of bluish purple color may be determined as positive in cells within a single well stained with the counter staining solution as described above.

Moreover, in connection with the process of the production of the probe for detection, reference may be made to Japanese Patent Nos. 2558420, 2798499, 2965543, 2965544, 3026789 and so on.

For example, for the culture through picking a microorganism from a working cell bank, the working cell bank (SA-24) is smeared by streaking with a platinum loop, a disposable plastic loop or the like on an L-broth solid medium containing 50 μg/ml ampicillin prepared in a sterile petri dish (microorganism picking).

Following overnight culture, a single colony is collected, and inoculated in 5 ml of an L-broth medium containing 50 μg/ml ampicillin, and then shaking culture is conducted overnight at 37° C. (preculture).

In a flask for culture including the medium described above in an amount of 400 ml is inoculated each 2.5 ml of the preculture liquid followed by shaking culture at about 37° C. overnight (regular culture).

Next, for extracting the SA-24 plasmid DNA, the culture liquid in the regular culture is centrifuged at 4° C. for 10 minutes at 4,000×g to collect the microorganism. The culture supernatant is removed, and thereto is added 20 ml of STE (10 mmol/l Tris-hydrochloric acid (pH 8.0), 1 mmol/l disodium ethylenediamine tetraacetate (EDTA), 0.1 mmol/l sodium chloride) to resuspend the cell bodies. Then, centrifugation is conducted at 4° C. for 10 minutes at 4,000×g to collect the microorganism. Thereto is added 5 ml of a solution-1 (50 mmol/l glucose, 25 mmol/l Tris-hydrochloric acid (pH 8.0), 10 mmol/l EDTA) containing 10 mg/ml lysozyme, and the cell are suspended therein followed by leaving to stand still at room temperature for 5 minutes. Thereto is added 10 ml of a solution-2 (0.2 mmol/l sodium hydroxide, 1% sodium dodecyl sulfate (SDS)) mixed by inversion and left to stand on ice for 10 minutes. Thereto is added 7.5 ml of an ice cold solution-3 (3 mol/l potassium acetate (pH 4.8)) mixed by inversion and left to stand on ice for 10 minutes.

After centrifugation by a high speed refrigerated centrifuge at 4° C. for 30 minutes at 45,00×g, the supernatant is recovered, and left to stand to cool to room temperature. After leaving to stand, 0.6 volume of isopropanol (about 24 ml) is added thereto, mixed by inversion and left to stand at room temperature for 5 minutes or longer. After centrifugation by a high speed refrigerated centrifuge at 25° C., for 30 minutes at 28,00×g, the supernatant is discarded, and thus resulting pellet is washed with 70% ethanol and air dried. After air drying, 8 ml of TE (10 mmol/l Tris-hydrochloric acid (pH 8.0), 1 mmol/l EDTA) is added thereto to dissolve the pellet (extraction of plasmid DNA).

Next, for the purification of the plasmid DNA containing SA-24, 800 μl of 10 mg/ml ethidium bromide and 8.6 g of cesium chloride are added to the resulting plasmid DNA followed by mixing by inversion to dissolve the plasmid. The solution is placed in a centrifuge tube, which is then capped or sealed. After centrifugation at 20° C. for 5 hours at 500,000×g with a vertical rotor, a band of the plasmid DNA is fractionated using a glass syringe or an injection needle under the irradiation of an ultraviolet ray light. To the fractionated plasmid DNA solution is added an equivalent amount of TE-saturated 1-butanol followed by mixing by inversion and centrifugation at 15,000×g for 5 minutes by a high speed microcentrifuge to remove the supernatant. This operation is repeated to eliminate ethidium bromide in the plasmid DNA solution. Next, thereto is added TE to give the volume of 1.5 ml followed by desalting on a demineralization column (NAP-10). To the desalted plasmid DNA solution is added 30 μl of a 3 mol/l sodium acetate solution followed by mixing, and 3 fold amount of 99.5% ethanol is added thereto followed by mixing by inversion and leaving to stand at −20° C. for 30 minutes or longer. After leaving to stand, centrifugation is conducted with a high speed refrigerated micro centrifuge at 4° C. for 20 minutes at 15,000×g to remove the supernatant. Thereafter, cold 70% ethanol is added thereto to suspend therein, and once again, centrifugation is conducted with a high speed refrigerated micro centrifuge at 4° C. for 20 minutes at 15,000×g to remove the supernatant. Thus resulting precipitate of the plasmid DNA is evaporated to dryness under a reduced pressure. TE in an amount of 100 μl is added to the plasmid DNA to dissolve completely, and the concentration is measured on the basis of the absorbance at 260 nm (Purification of plasmid DNA containing SA-24). Then, size check of the plasmid DNA containing SA-24 is carried out by a treatment with arestriction enzyme and agarose electrophoresis.

For conducting purification of SA-24 by the treatment of the plasmid DNA containing SA-24 using a restriction enzyme and agarose electrophoresis, 1 mg of the plasmid DNA containing SA-24 after finishing the check of the molecular weight is combined with a restriction enzyme HindIII alone or with other restriction enzyme, and is digested by the reaction at 37° C. for 1.5 hours. Following the digestion of the plasmid DNA, a part of the reaction liquid is electrophoresed on a 0.8% agarose to ascertain that the digestion is completely terminated. After confirming the digestion, a band of SA-24 is recovered through the electrophoresis on a 0.8% preparative agarose gel. Thus recovered SA-24 is extracted from the agarose gel and purified, and the concentration is measured with an absorbance meter. A part of the purified SA-24 is electrophoresed on a 0.8% agarose gel to verify that a single band is found. For labelling SA-24, 2 μg of the purified SA-24 is used, and may be subjected to digoxigenin labelling in a reaction liquid having the composition described in Table 1 below.

TABLE 1
Composition of the reaction liquid for labeling

| | Amount included (μL) |
|---|---|
| DNA probe | X |
| 10 × L.B.^{(a)} | 10 |
| 100 mmol/L dithiothreitol | 10 |
| dNTps^{(b)} (A, G, C: 0.5 mmol/L) | 4 |
| digoxigenin-dUTP^{(c)} (0.4 mmol/L) | 5 |
| DNase I^{(d)} | 6 |
| 10 U/μL DNA polymerase I | 2 |
| Sterile purified water | Y |
| Total | 100 |

[explanatory notes]
^{(a)}10 × L.B.: 0.5 mol/L Tris-hydrochloric acid (pH 7.5), 50 mmol/L magnesium chloride, 0.5 mg/mL bovine serum albumines
^{(b)}dNTPs: 0.5 mmol/L 2′-deoxyadenosine-5′-triphosphate, 0.5 mmol/L 2′-deoxyguanosine-5′-triphosphate, 0.5 mmol/L 2′-deoxycytidine-5′-triphosphate
^{(c)}digoxigenin-dUTP: 0.4 mmol/L digoxigenin-11-2′-deoxyuridine-5′-triphosphate
^{(d)}DNase I: deoxyribonuclease I is diluted in a solution of 25 mmol/L Tris-hydrochloric acid (pH 7.5) and 50% glycerin such that the amount of 50 to 150 mU per total volume of 100 μl is used to give the aforementioned amount included.

In Table 1, X represents the volume which may be added such that preferred concentration of the probe as described above is provided depending upon the concentration of the probe stock solution, and the amount Y of purified water is determined following this volume to adjust the final volume.

After the labelling, 100 μl of TE is added to the reaction liquid to terminate the reaction. The reaction terminated solution is poured into a spin column, and centrifuged at 4° C. for 10 minutes at 380×g to remove free nucleotides. Next, the concentration of the eluate is measured with an absorbance meter, and then adjusted to give 10 ng/μl with TE.

In order to verify the labelling, 0.5 μl of the labelled SA-24 is dropped onto a membrane, and air dried. The membrane is immersed in a blocking reagent, and blocked at room temperature for 30 minutes. In an alkaline phosphatase labelled anti-digoxigenin antibody solution diluted to 5,000 fold in 0.1 mol/l Tris-hydrochloric acid (pH 7.5) and 0.15 mol/l sodium chloride, is immersed the membrane at room temperature for 30 minutes. The membrane is immersed in 0.1 mol/l Tris-hydrochloric acid (pH 7.5), 0.15 mol/l sodium chloride, and washed twice by shaking at room temperature for 10 minutes. In 0.5 mol/l Tris-hydrochloric acid (pH 9.5), 0.15 mol/l sodium chloride and 50 mmol/l magnesium chloride is immersed the membrane at room temperature for 10 minutes. The membrane is immersed in the coloring reagent at room temperature under light shielding for 10 minutes. The membrane is immersed in TE to terminate the color development. Verification of the labelling is executed -by the observation of bluish purple coloring at the potion under the spotting. For producing the spin column, a small amount of sterilized glass wool is packed in a 1 ml disposable syringe. Sephadex G-50 swelled with 1 mmol/l Tris-hydrochloric acid (pH 7.5), 1 mmol/l EDTA and 0.1% SDS is filled in the syringe. The syringe is placed into a 15 ml disposable conical tube followed by centrifugation at 4° C. for 10 minutes at 320×g to throw the excess buffer away. The syringe is drawn from the disposable conical tube, and after discarding the excreted buffer, the spin column is produced by placing the syringe on the bottom of a disposable conical tube which had been a 1.5 ml Eppendorf tube placed therein.

To determine the specificity of the probe, dot blot hybridization may be carried out according to the following procedure.

First, for the denaturation of each spotted genomic DNA, each 100 ng of various types of bacterial genomic DNA as prepared is spotted to a nylon membrane (Pall Biodyne® type B, manufactured by Nihon Pall Ltd.) on a filter paper (manufactured by Whatman, 3 MM) saturated with a solution containing 0.5 mol/l sodium hydroxide and 1.5 mol/l sodium chloride according to a conventional process, and the air dried membrane is left to stand for 10 minutes. Next, the membrane is allowed to stand still on the filter paper described above which is saturated with a solution containing 0.5 mol/l Tris-hydrochloric acid (pH 7.5) and 1.5 mol/l sodium chloride for 10 minutes to neutralize the denaturated DNA. Furthermore, it is left to stand still on the filter paper as described above which is saturated with a 2×SSC (Standard Saline Citrate) solution for 5 minutes followed by rinsing. Thereafter, the membrane is air dried, immersed in a 2×SSC solution and allowed for infiltration for 5 minutes. According to a conventional process, the membrane is immersed in a prehybridization solution within a plastic bag, and affinitized at 42° C. for 60 minutes. The membrane is immersed in 15 ml of a hybridization solution containing 400 mg of the probe in the plastic bag, and the reaction is allowed at 42° C. overnight. Next, the membrane is immersed in a solution containing 2×SSC and 0.1% SDS (sodium dodecyl sulfate), and washed for 5 minutes (repeated twice). Thereafter, the membrane is immersed in a solution containing 0.1×SSC and 0.1% SDS, and washed at 60° C., for 10 minutes (repeated three times). The membrane is then immersed in a 2×SSC solution, and washed for 5 minutes. The membrane is immersed in a solution containing 3% bovine serum albumines, 1% blocking buffer (manufactured by Boeringer), 0.1 mol/l Tris-hydrochloric acid (pH 7.5) and 0.15 mol/l sodium chloride, and is gently shaken for 30 minutes. Thereafter, the membrane is immersed in a solution of alkaline phosphatase labelled anti-digoxigenin antibody (manufactured by Boeringer) diluted to 5,000 fold in a solution containing 0.1 mol/l Tris-hydrochloric acid (pH 7.5) and 0.15 mol/l sodium chloride, and is gently shaken for 30 minutes. Next, the membrane is immersed in a solution containing 0.1 mol/l Tris-hydrochloric acid (pH 7.5) and 0.15 mol/l sodium chloride, and is shaken for 15 minutes (twice). The membrane is immersed in a solution containing 0.1 mol/l Tris-hydrochloric acid (pH 9.5), 0.1 mol/l sodium chloride and 5 mmol/l magnesium chloride, and is shaken for 5 minutes. The membrane is immersed in an NBT-BCIP solution (manufactured by GIBCO BRL), and the color development reaction is allowed under light shielding. In TE (10 mmol/l Tris-hydrochloric acid (pH 8.0), 1 mmol/l EDTA) is immersed the membrane to terminate the color development reaction, and is air dried. The prehybridization solution and the hybridization solution are as shown in Table 2 below.

TABLE 2
[represented by ml]

| | Prehybridization | Hybridization |
|---|---|---|
| | solution | solution |
| Formamide | 7.5 | 6.75 |
| 20 × SSC solution | 3.75 | 3.75 |
| 100 × Denhardt's solution | 0.75 | 0.15 |
| 0.5 mol/L phosphate buffer | 0.75 | 0.6 |
| sterile purified water | 1.5 | 1.95 |
| 10 mg/mL salmon sperm DNA | 0.75 | 0.3 |
| 50% dextran sulfate | — | 1.5 |
| Total liquid volume | 15.0 | 15.0 |

The surfactant which may be used in the step of in situ hybridization is any of known surfactants. Surfactants are generally classified in anion surfactants, nonionic surfactants, cation surfactants and ampholytic surfactants.

Anion surfactants are also referred to as anionic surfactants, which yield an organic anion upon ionization in water. When a lipophilic group in the molecule of the surfactant is represented by R, examples of the anion surfactant include RCOONa, RSO₃Na, RSO₄Na and the like. An aqueous solution of the surfactant containing a weakly acidic group such as RCOONa is liable to be hydrolyzed and is weak alkaline. However, an aqueous solution of a surfactant having a strongly acidic group such as RSO₃Na, RSO₄Na or the like is resistant to hydrolysis, which shall be neutral. Because it is anionic, it may lose surface activity in the presence of a large quantity of cationic substance, and may be inactivated in a strongly acidic circumstance.

Nonionic surfactants refer to those having a hydrophilic group which is nonionic. An ethylene oxide group (—CH₂CH₂O—) is often used as the hydrophilic group. As number of this group increases, hydrophilicity is increased. To the contrary, as number of the lipophilic group increases, lipophilicity is increased. Therefore, it is characterized in that surfactants with variously altered hydrophilicity and lipophilicity can be obtained. Because a nonionic surfactant does not ionize in water and is hardly affected by inorganic salts, less action is exerted also on a living body. In addition, the detergent action thereof is potent with comparatively less foaming, therefore, it is widely used not alone as a detergent, but in pharmaceuticals, cosmetics, foods and the like. Water soluble nonionic surfactant becomes insoluble in water at a certain temperature as the temperature rises, and then the aqueous solution starts to be turbid. Such turbidity results from the cleavage of hydrogen bonds between the hydrophilic groups and water.

Cation surfactants are also referred to as cationic surfactants, which yield an organic cation upon ionization in water. Although cation surfactants do not have potent detergent action in general, they strongly bind to anionic substances such as bacteria, leading to a great bactericidal action. Moreover, they also have an anti-static ability for fibers and plastics. Although dodecyltrimethyl chloride [C₁₂H₂₅(CH₃)₃N]Cl as a typical exemplary cation surfactant is water soluble, didodecyldimethylammonium chloride [(C₁₂H₂₅)₂(CH₃)₂N]C1 is insoluble in water, which forms a vesicle in the form of a bimolecular film in water, and is soluble in benzene.

Ampholytic surfactants are surfactants having both an anionic group and a cationic group in the molecule. Ionization state thereof in water is similar to those of amino acids, and thus many of ampholytic surfactants are amino acid derivatives. Therefore, they have an isoelectric point similarly to amino acids, which act as an anion surfactant in an alkaline region from the isoelectric point, whilst as a cation surfactant in an acidic region. Water solubility becomes the lowest at the isoelectric point, and the surface tension is also reduced. Ampholytic surfactants are used for a bactericide, an antistatic agent or the like.

Furthermore, anion surfactants are classified into the carboxylic acid type, sulfonic acid type, sulfate ester type and phosphate ester type, whilst nonionic surfactants are classified into the ester type, ether type, ester ether type and alkanolamide type. Cation surfactants are classified into alkylamine salt type and quaternary ammonium salt type, whilst ampholytic surfactants are classified into carboxy betaine type, 2-alkylimidazoline derivative type and glycine type.

Moreover, the anion surfactants of carboxylic acid type are further classified into fatty acid monocarboxylate salts, N-acylsarcosine salts and N-acylglutamate salts. Representative examples thereof respectively include: sodium laurate and medicated soap as the fatty acid monocarboxylate salts; sodium N-lauroylsarcosine as the N-acylsarcosine salt; and disodium N-lauroylglutamate as the N-acylglutamate. Still more, the sulfonic acid type is further classified into dialkyl sulfosuccinate salts, alkane sulfonate salts, alpha-olefin sulfonate salts, straight chain alkyl benzenesulfonate salts, alkyl (branched chain) benzenesulfonate salts, alkyl naphthalenesulfonate salts, naphthalenesulfonate salts-formaldehyde condensates and N-methyl-N-acyltaurine salts. Representative examples include: sodium dioctyl sulfosuccinate as the dialkyl sulfosuccinate salt; sodium dodecane sulfonate as the alkane sulfonate; sodium straight chain dodecyl benzenesulfonate as the straight chain alkyl benzenesulfonate salt; sodium dodecyl benzenesulfonate as the alkyl (branched chain) benzenesulfonate salt; sodium butyl naphthalenesulfonate as the alkyl naphthalenesulfonate salt; and sodium N-methyl-N-stearoyltaurine as the N-methyl N-acyltaurine salt. In addition, the sulfate ester type is further classified into alkyl sulfate salts, polyoxyethylene alkyl ether sulfate salts and oil-and-fat sulfate ester salts. Representative examples include sodium dodecyl sulfate, sodium lauryl sulfate and sodium cetyl sulfate as the alkyl sulfate salt; and polyoxyethylene lauryl ether sulfate triethanolamine as the polyoxyethylene alkyl ether sulfate salt. Moreover, the phosphate ester type is further classified into alkyl phosphate salts, polyoxyethylene alkyl ether phosphate salts and polyoxyethylene alkylphenyl ether phosphate salts. Representative examples include disodium monolauryl phosphate as the alkyl phosphate salt; and sodium polyoxyethylene lauryl ether phosphate and polyoxyethylene oleyl ether phosphate (8 MOL) as the polyoxyethylene alkyl ether phosphate salt.

Ester type of the nonionic surfactants is further classified into fatty acid glycerin, fatty acid sorbitan and fatty acid sucrose ester. Representative examples respectively include: glycerin monostearate as the fatty acid glycerin; sorbitan monostearate, sorbitan trioleate, sorbitan sesquioleate, sorbitan monolaurate, polysorbate 20 (polyoxyethylene sorbitan fatty acid ester), polysorbate 60 and polysorbate 80 as the fatty acid sorbitan; and stearic acid sucrose ester as the fatty acid sucrose ester. Additionally, the ether type is further classified into polyoxyethylene alkyl ether, polyoxyethylene alkyl phenyl ether and polyoxyethylene polyoxypropylene glycol. Representative examples include: polyoxyethylene lauryl ether, polyoxyethylene stearyl ether and polyoxyethylene cetyl ether as the polyoxyethylene alkyl ether; and polyoxyethylene nonyl phenyl ether and polyoxyethylene octyl phenyl ether as the polyoxyethylene alkyl phenyl ether. In addition, the ester ether type is further classified into fatty acid polyethylene glycol and fatty acid polyoxyethylene sorbitan. Representative examples thereof respectively include oleic acid polethylene glycol as the fatty acid polyethylene glycol; and polyoxyethylene sorbitan palmitate and polyoxyethylene sorbitan monolaurate as the fatty acid polyoxyethylene sorbitan. In addition, the alkanolamide type involves only fatty acid alkanolamide alone. Representative example is lauric diethanolamide.

The alkyl amine salt type of the cation surfactant includes monoalkyl amine salts, dialkyl amine salt and trialkyl amine salts. Representative examples thereof include monostearyl amine hydrochloride. Moreover, the quaternary ammonium salt type is further classified into alkyltrimethyl ammonium chloride (or bromide or iodide), dialkyldimethyl ammonium chloride (or bromide or iodide), and alkyl benzalkonium chloride. Representative examples respectively include: stearyltrimethyl ammonium chloride as the alkyltrimethyl ammonium chloride (or bromide or iodide); distearyldimethyl ammonium chloride as the dialkyldimethyl ammonium chloride (or bromide or iodide); and lauryl benzalkonium chloride as the alkyl benzalkonium chloride.

The carboxy betaine type of the ampholytic surfactant is only alkyl betaine alone. Representative example is lauryl betaine. Additionally, the 2-alkyl imidazoline derivative type is only 2-alkyl-N-carboxymethyl-N-hydroxyethyl imidazolinium betaine alone. Representative example includes 2-undecyl-N-carboxymethyl-N-hydroxyethyl imidazolinium betaine. In addition, the glycine type may be alkyl (or dialkyl) diethylene triaminoacetic acid, and the representative example includes dioctyl diethylene triaminoacetic acid.

Moreover, in addition to the representative examples as described above, Triton X-100, lauryl sarcosine, saponin, BRIJ35, alkyl allyl polyether alcohol, higher alcohol sulfate, N-cocoyl-L-arginine ethyl ester DL-pyrrolidone carboxylate salt, sodium N-cocoyl-N-methyl aminoethyl sulfonate, cholesterol, self emulsifying type monostearate glycerin, squalane, stearyl alcohol, stearic acid polyoxyl 40, cetyl alcohol, cetomacrogol 1000, sebacate diethyl, nonylphenoxy polyoxyethylene ethane sulfate ester ammonium, polyoxyethylene oleylamine, polyoxyethylene sorbit yellow bees wax, polyoxyl 35 castor oil, macrogol 400, N-coconut oil fatty acid acyl L-arginine ethyl.DL-pyrrolidone carboxylate salt, lauryldimethylamine oxide solution, lauromacrogol, methylcellulose, CMC (carboxymethylcellulose), polyoxyethylene hardened castor oil 20 and polyoxyethylene hardened castor oil 60, CHAPS, deoxycholic acid, digitonin, n-dodecyl maltoside, Nonidet P40, n-octyl glucoside, octyl thioglucoside, laurate sucrose, dodecyl poly(ethylene glycol ether)n,n-dodecyl-N,N-dimethyl-3-ammonio-1-propanesulfonate and the like are also included.

Various surfactants as listed above are essentially used in the step of in situ hybridization, but the process for use is not particularly limited. For example, the surfactant may be admixed in the probe solution or probe dilution solution, alternatively, a solution containing the surf actant which was separately prepared from the probe solution may be added prior to, concurrently with or later than coating of the probe solution on the smear site. Such a process may be altered ad libitum by the person skilled in this art.

In the present invention, when a positive control probe is required, it can be produced as follows. For example, in order to conduct the extraction and purification of the genomic DNA of U937 cell (ATCC CRL-1593.2), U937 cells are first cultured in a 5% carbon dioxide gas incubator at 37° C. using an RPMI1640 medium (25 ml) in a cell culture flask (175 cm²). The U937 culture solution is placed in a 50 ml centrifuge tube, and centrifuged at 4° C. for 10 minutes at 220×g to recover the U937 cells. The cells are suspended and washed in 10 ml of PBS, and again centrifuged at 4° C. for 10 minutes at 180×g to recover the cells. Thereafter, the supernatant is discarded, and the cells are suspended in 1 ml of a TE solution containing 200 μg/ml proteinase K and containing 1% SDS, followed by leaving to stand at 37° C. for 30 minutes. Phenol extraction is repeated three to four times to execute deproteinization. Genome deposited through the ethanol precipitation is recovered, dissolved in 500 μl of sterile purified water containing 2.5 μg of ribonuclease, and left to stand at 42° C. for 30 minutes.

The phenol extraction is repeated two to three times to execute deproteinization. Genome deposited through the ethanol precipitation is recovered, and dissolved in 500 μl of TE. Thereafter, a positive control probe can be produced by measuring the concentration with an absorbance meter, and subjecting to digoxigenin labelling. Moreover, the positive control probe which may preferably used is one which permits to ascertain the hybrid formation when the positive control probe is subjected to dot hybridization on a membrane with 100 ng of U937 genome spotted thereon. When a negative control probe is required, it can be produced by any known method.

Preparation of Digested Sample

Specific process for producing a phagocyte post phagocytosis of the present invention (hereinafter, referred to as digested sample ) is illustrated below.

Materials for use which are required include U937 cell (human monocyte established cell: ATCC CRL-1593.2), Staphylococcus aureus (ATCC 126000), Staphylococcus epidermidis (ATCC 14990), Pseudomonas aeruginosa (ATCC 10145), Enterococcus faecalis (ATCC 19433), Escherichia coli (ATCC 11775), heparinized healthy human blood, brain heart infusion (BHI) (manufactured by DIFCO), RPMI 1640 (RPMI medium 1640 (manufactured by GIBCO)) containing Fetal Bovine Serum (final concentration: 10%, manufactured by GIBCO) and Antibiotic-Antimycitic (final concentration: 1%, manufactured by GIBCO) and the like.

Instruments for use which are required include a carbon dioxide gas incubator (manufactured by Tabai Espec Corporation: BNA-121D type), a. low speed refrigerated centrifuge (manufactured by Beckman: CS-6KR type), a counting chamber (manufactured by Elmer: bright line type), a shaking incubator (manufactured by TIETECH Co., Ltd.: BR-300L type), an absorbance meter (manufactured by Beckman: DU68 type), an incubator (manufactured by Yamato Scientific Co., Ltd.: IC-62 type), an incident-light inverted microscope (manufactured by Nikon: DIAPHOT type), a fluorescence microscope (manufactured by Nikon: OPTIPHOT type), a CCD camera (manufactured by Hamamatsu Photonics KK.: C5810-01 type) and the like.

First, for preparing U937 cells, U937 cells (human monocyte established cell: ATCC CRL-1593.2) are cultured in an RPMI 1640 medium within a cell culture flask (e.g., 175 cm²) in a 5% carbon dioxide gas incubator at about 20 to about 40° C., preferably at about 37° C. The cell culture flask is preferably one including a material consisting of a component which is liable to adhere to cells. Next, the U937 cell culture liquid is placed in a centrifuge tube, and centrifuged at 0° C. to about 10° C., preferably at about 4° C. for about 10 minutes at about 150 to about 350×g, preferably about 220×g to recover the U937 cells. Then, thus recovered U937 cells are suspended in PBS, and the cell number is counted with a counting chamber. The cell number may be adjusted to about 1×10⁴ cells/μl to 2×10⁴ cells/μl.

For preparing a bacterial digested sample, Staphylococcus aureus, Staphylococcus epidermidis, Pseudomonas aeruginosa, Enterococcus faecalis and Escherichia coli are inoculated in a BHI culture liquid (supra), and cultured at about 20 to about 40° C., preferably at about 37° C. for 6 hours or longer. The cultured bacterial liquid is centrifuged at 0 to about 10° C., preferably at 4° C., for example, at 2,000×g for about 10 minutes to collect the bacteria. After discarding the supernatant, it is preferred that pellet of the bacteria is suspended using PBS, and centrifuged once again at 4° C. for 10 minutes at 2,000×g to collect the bacteria. After suspending thus collected bacteria in PBS, they may be diluted in PBS to produce a bacterial liquid prepared such that it has the turbidity (O.D.=600 nm) of about 0.001 to about 0.1, preferably about 0.01 to about 0.03, and in particular, about 0.01 to about 0.03 for Staphylococcus aureus, about 0.01 to about 0.03 for Staphylococcus epidermidis, about 0.02 to about 0.03 for Pseudomonas aeruginosa, about 0.01 to about 0.03 for Enterococcus faecalis, and about 0.02 to about 0.03 for Escherichia coli, respectively, when measured with an absorbance meter. Thus produced bacterial liquid is transferred to a discrete culture flask, and left to stand still for about 30 minutes at room temperature. Heparinized healthy human blood is collected, and thereto is added the aforementioned reagent for separating hemocyte at a ratio of approximately 4:1, and left to stand still at about 20 to about 40° C., preferably at about 37° C. for 30 minutes to yield the leukocyte fraction. Thus obtained leukocyte fraction is suspended in PBS. The supernatant in the culture flask is gently discarded, and the leukocyte fraction diluted in PBS is added to the flask followed by leaving to stand still at room temperature for about 10 minutes. The supernatant in the culture flask is discarded, and the leukocytes attached to the bottom of the flask are recovered in a centrifuge tube with PBS containing 0.02% EDTA, and centrifuged e.g., at 4° C. for 10 minutes at about 140 to about 180×g to collect the leukocytes. When contamination of erythrocytes is found in the collected leukocytes, precipitates of the leukocytes are gently suspended in sterile purified water to allow hemolysis, subjected to isotonization through adding PBS, followed by centrifugation once again at 4° C. for 10 minutes at about 140 to about 180×g to collect the leukocytes. The collected leukocytes are suspended in PBS, and cell number is counted with a counting chamber to adjust to give about 1×10⁴ cells/μl to about 5×10⁴ cells/μl. These digested samples are referred to as SA digested sample, SE digested sample, PA digested sample, EF digested sample and EK digested sample.

For conducting the smear fixation, the prepared U937 cells and each bacterial digested sample produced as described above is smeared on each well of an APS coated slide glass followed by air drying.

It is preferred that cell number of each bacterial digested sample smeared and fixed on the slide glass is about 5.0×10⁴ to about 2.5×10⁵ cells/well, while cell number of U937 cells is about 5.0×10⁴ to about 1.0×10⁵ cells/well. For the fixation, the sample is immersed in Carnoy's fixative(supra) for 20 minutes and thereafter immersed in 75% ethanol for 5 minutes. After washing Carnoy's fixative and air drying, the sample may be stored at 4° C. until use in the test.

Measurement of the phagocytosis rate is executed by staining the bacterial digested sample smeared and fixed on the slide glass with an acridine orange staining solution, and counting about 200 cells randomly with a fluorescence microscope (×1,000). Among the measured cells, cells including bacteria phagocytized within the cells are determined as positive cells, and the phagocytosis rate (%) is calculated according to the mathematical formula below.
Phagocytosis rate (%)=[(Positive cell number/Measured cell number)×100]

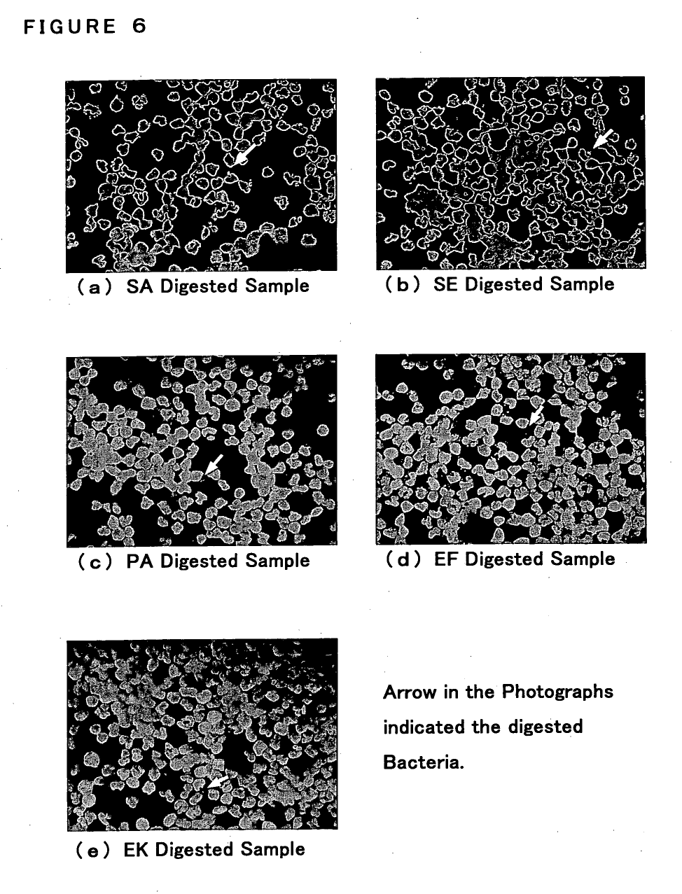

FIG. 6 illustrates the state of the phagocytes prepared and observed by microscopic examination. Specific operation process involving Carnoy fixation, treatment for promoting permeability of the leukocyte cell membranes, lytic treatment, acetylation of the cell membrane protein, alkaline treatment of DNA of the bacterial body, in situ hybridization, blocking, reaction with labelled antibody, detection, and determination, which may be employed is as described herein.

Further, the present invention also includes a kit for evaluating a phagocytotic function which comprises fixing phagocytes post phagocytosis of a foreign microorganism, executing a treatment for promoting permeability of the cell membranes of the phagocytes, executing a treatment for exposing the DNA of the foreign microorganism existing in the phagocytes, carrying out in situ hybridization using a DNA probe for detection capable of hybridizing with the DNA under a stringent condition in the presence of a surfactant; and evaluating the phagocytotic function by the resulting signal, the kit having (1) the foreign microorganism, (2) at least one or more enzyme selected from the group consisting of lysostafin, lysozyme, N-acetylmuramidase and zymolase used in the exposing step of the DNA, and (3) one or more DNA probe for detection.

This kit includes, reagent for separating blood, enzyme pretreatment reagent, enzyme reagent, acetylation reagent, probe solution, blocking reagent, labelled antibody, labelled antibody diluent, coloring pretreatment liquid-1, coloring pretreatment liquid-2, coloring reagent, counter staining solution, PBS stock solution, hybridization stock solution, labelled antibody washing solution, coloring reagent washing solution, APS coated slide glass, probe dilution solution, buffer A and the like as demonstrated in the following Examples. Among these, it is preferred that at least the enzyme reagent and the probe solution are included. In addition, various reagents used in the present invention may be included for example, chloroform, ethanol, acetic anhydride, DMSO, PMSF, formamide, acetic acid, hydrochloric acid, sodium hydroxide and the like. Moreover, instrument and machine such as low speed centrifuge, incubator, counting chamber, shaker, humid box, incubator, light microscope, variable pipette, blood collection tube, tip, pipette, staining bottle, measuring cylinder, glass syringe, 0.2 μm syringe top filter may be included.

Furthermore, the present invention provides a process for monitoring the gene of a foreign microorganism phagocytized by a phagocyte included in a clinical specimen which contains a phagocyte derived from a living body.

Moreover, the present invention provides a process for identifying the gene of a microorganism which becomes a candidate of the causative microorganism which a causative microorganism of sepsis or a causative microorganism of bacteremia is specified on the basis of the results identified.

The clinical specimen which may be used herein is a clinical specimen which contains a phagocyte derived from a living body, and examples thereof include body fluids such as blood, tissue fluid, lymph fluid, cerebrospinal fluid, pyo, mucus, snot, sputum and the like. Additionally, in compliance with the disease states such as diabetes, renal disorder, hepatic disorder or the like, phagocytes derived from the living body may be included in urine, ascites, dialysis drainage and the like as well as in lavage obtained after washing nasal cavity, bronchial tube, skin, various organs, bone or the like, therefore, these may be used as the clinical specimen according to the present invention. In addition, tissues such as skin, lung, kidney, mucosa and the like may be used as the clinical specimen. Because a macrophage which is one of the phagocytes varies to several forms such as monocyte, pulmonary alveolus macrophage, peritoneal cavity macrophage, fixed macrophage, free macrophage, Hansemann macrophage, inflammatory macrophage, liver Kupffer cell, brain microglia cell, not only blood but also tissues including these cells can be used as the clinical specimen of the present invention. For example, a causative microorganism of nephritis can be detected and identified through obtaining the renal tissue from a patient suspected as suffering from nephritis by kidney biopsy, obtaining phagocytes which are present in the tissue by detaching the cells using an enzyme such as trypsin or the like, and using thus resulting phagocytes.

It was revealed that when this process was applied in practice to diagnoses for blood of a variety of patients suspected as suffering from sepsis, causative microorganism could be detected with about 4 times higher sensitivity compared to the blood culture process with no influence of the administered antimicrobial agent, and the identity of the detected microorganism strain was favorable. Furthermore, in comparison with the blood culture which requires 3 days or longer and approximately 14 days for the examination, an accurate result can be achieved by a simple operation within a short time period, i.e., about 8 hours, until the completion of the entire operation, according to the process of the present invention. Therefore, a useful marker can be provided in the monitoring and the like in prognosis or diagnosis of an infectious disease such as sepsis, bacteremia or the like in which a rapid and favorable care is required, in particular.

According to one embodiment of the present invention, a performance test is provided which is characterized in that a phagocyte post phagocytosis of a foreign microorganism is used, and examples of the test include sensitivity tests, specificity tests, reproducibility tests and the like of a kit for evaluating a phagocytotic function. In these tests, a phagocyte post phagocytosis of a foreign microorganism can be used as a positive control. When a digested sample is used in the performance test for Staphylococcus aureus, particularly in a sensitivity test, it may be defined that a signal can be detected when the test is performed according to the in situ hybridization process described herein using a digested sample of Staphylococcus aureus.

Additionally, upon performing a specificity test, it may be defined that a signal can be detected for Staphylococcus aureus alone, when the test is performed according to the in situ hybridization process described herein using various bacterial digested samples.

Further, upon performing a reproducibility test, it may be defined that the achieved results are identical when the specificity test is performed by concomitantly repeating three tests. Also in respect of other bacteria, e.g., Staphylococcus epidermidis, Pseudomonas aeruginosa, Enterococcus faecalis, Escherichia coli, Enterobactor cloacae and Klebsiella pneumoniae, definition may be made with reference to the performance tests as described above.

Moreover, when a digested sample is used as a positive control in the performance test such as sensitivity test, specificity test, reproducibility test or the like as described above, in connection with the standard of the digested sample and the process for testing, cell number smeared and fixed on the slide glass of each bacterial digested sample is preferably about 5.0×10⁴ to about 2.5×10⁵ cells/well, whilst cell number of U937 cells is preferably about 5.0×10⁴ to about 1.0×10⁵ cells/well.

Moreover, upon measurement of the phagocytosis ratio, specific morphology of a phagocyte can be observed as shown in FIG. 6, when a bacterial digested sample smeared and fixed on the slide glass is stained with an acridine orange staining solution, and about 200 cells are randomly counted with a fluorescence microscope (×1,000). Accordingly, cells including bacteria phagocytized within the cells are determined as positive cells among the measured cells, and the phagocytosis rate (%) is calculated.
Phagocytosis rate (%)=[(Positive cell number/Measured cell number)×100]

In the process for evaluating phagocytotic function for a foreign microorganism, evaluation may be made by not only the signal obtained by carrying out in situ hybridization, but also for example, calculation through employing the phagocytosis rate as described above. Hence, the process for evaluating a phagocytotic function can be performed on the basis of the morphologic observation by the in situ hybridization process and staining. Such an evaluation process can be also utilized in a process for evaluating an immune function of a living body, a process for evaluating differentiation efficiency into a phagocyte, a process for evaluating a modulator against a phagocytotic function, a process for screening, a process for the clinical test to examine a dosage regimen of an agent.

Suitable immune function may be a phagocytotic ability for a living microorganism by a leukocyte, in particular, a phagocytotic ability for a living microorganism by a leukocyte of a patient after the radiation exposure or the administration of an anticancer agent. For example, when a certain agent is administered intending to promote or antagonize a function of a phagocyte such as potentiation of a declined immune system accompanied by the administration of a chemotherapeutic agent in a cancer therapy, suppression of a rejection symptom upon organ transplantation, and the like, this process can ascertain whether or not the agent effectively acts in vivo actually. Therefore, a useful guideline can be provided for the selection of a drug or a dosage.

Additionally, the process of the present invention has an effect to contribute to a basic study and a clinical study in regard to the interaction between a microorganism in the field of bacteremia and a phagocyte, and may be also employed in the determination of effectiveness of a modulator of a phagocytotic function or in the screening of a novel substance having a modulatory action against a phagocytotic function. Also in this process, the aforementioned process for evaluating a phagocytotic function is utilized, therefore, substantial effects by a modulator such as an agonist or an antagonist toward a subject can be assessed with higher reliability than any conventional process.

Further, because effects on a certain individual by a modulator which may cause great individual differences in terms of the effectiveness, side effects and the like can be identified, it may be helpful in the determination of a medical guideline in an order made fashion which is suited to each patient. In other words, a clinical testing process is provided which is characterized in: obtaining phagocytes from a subject prior to and following the administration of an agent to the subject; evaluating a function of the phagocyte by the process as described above; and examining a dosage regimen of the agent judging from the effect of the agent determined on the basis of the evaluation result.

The modulator is not limited as long as it is a substance which directly or indirectly participates in a phagocyte, for example, a substance which promotes or suppresses the differentiation of a phagocyte, a substance which promotes or suppresses a phagocytotic function, or the like. Examples thereof include G-CSG, anticancer agents, antibiotics, immune function activators, leukocyte differentiation factors and the like.

### EXAMPLES

Although the present invention is specifically explained by way of Examples below, as a matter of course, the disclosure of these Example should not be construed as limiting the present invention.

Example 1

### Collection of Blood, Treatment of Blood Specimen

As clinical specimens, 12 specimens of blood collected from patients suspected as suffering from sepsis (specimens A to L) were used. Ten ml of heparinized venous blood was collected from each patient, and after admixing the blood with a reagent for separating blood (225 mg of sodium chloride, 1.5 g of dextran (MW: 200,000-300,000)), adjusted to give the total volume of 25 ml with sterile purified water) at a ratio of 4:1, a leukocyte fraction (upper layer) was obtained by leaving to stand still at 37° C. for 30 minutes. Leukocytes were obtained by centrifugation of the resultant leukocyte fraction at 4° C. for 10 minutes at 160×g. Next, 1 ml of sterile purified water was added to thus resulting pellet of the leukocytes and suspended, and immediately thereafter an excess amount of PBS (18.24 g of sodium chloride, 6.012 g of sodium monohydrogen phosphate 12 hydrate, 1.123 g of sodium dihydrogen phosphate dihydrate, adjusted to give the total volume of 120 ml with sterile purified water (PBS stock solution) diluted to 20 fold with sterile purified water)was added thereto to result in isotonization, followed by centrifugation once again at 4° C. for 10 minutes at 160×g.

### Example 2

### Fixation of Leukocytes

An APS coated slide glass was used which is a slide glass (manufactured by JAPAN AR BROWN CO., LTD., item number: MS311BL) with 3-aminopropyltriethoxysilane (APS, SIGMA) coated thereon. For producing the APS coated slide glass, a slide glass (item number: MS311BL) was first fixed on a slide holder, and thereafter was washed by immersing in a diluted neutral detergent for 30 minutes, and the detergent is sufficiently removed with running water. Next, the slide glass was washed with purified water and sufficiently dried at high temperature (100° C. or greater) followed by leaving to stand to cool at room temperature. Then, this slide glass was immersed in acetone containing 2% APS for 1 minute, and immediately thereafter washed briefly with acetone and sterile purified water sequentially followed by air drying. In addition, after conducting the operation once again of immersing the slide glass in acetone containing about 2% APS for 1 minute, followed by immediate and brief washes with acetone and sterile purified water in a sequential manner and air drying, the APS coated slide glass was produced by drying at 42° C.

Leukocyte cell number of the leukocyte fraction is measured using a counting chamber after adding a small amount of PBS to the leukocytes pellet obtained by centrifugation at 4° C. for 10 minutes at 160×g followed by suspending therein. Leukocytes were supported on the APS coated slide glass by smearing 5 μl of the leukocyte suspension, which was prepared to yield the cell number of 1×10⁵ cells/well with PBS, on each well of the APS coated slide glass such that the leukocytes are spread over to give a single layer, and completely air drying. Thereafter, the slide glass was immersed in Carnoy's fixative (a mixed solution at a volume ratio of ethanol:chloroform acetic acid=6:3:1) for 20 minutes, then immersed in 75% ethanol solution for 5 minutes, and completely air dried.

### Example 3

The slide glass was immersed in PBS for 10 minutes, and thereafter, in a solution of an enzyme pretreatment reagent (prepared by mixing 1.25 g of saponin, 1.25 ml of t-octylphenoxypolyethoxyethanol (specific gravity: 1.068 to 1.075 (20/4° C.), pH (5 w/v %) 5.5-7.5) and 25 ml of the PBS stock solution, and adjusting to give the total volume of 50 ml with sterile purified water) diluted to 10 fold in sterile purified water, and allowing infiltration on a shaker for 10 minutes.

Example 4

### Enzymatic Lysis Treatment of Wall of Bacterial Body

In order to expose the DNA of a causative microorganism of an infectious disease, an enzyme reagent solution was prepared by adding 1 ml of an enzyme reagent dissolving solution (prepared by 100 fold dilution of dimethylsulfoxide (DMSO) which contains 0.1 mol/l phenylmethylsulfonylfluoride (PMSF) in PBS) to an enzyme reagent (N-acetylmyramidase 1,000 units/ml, lysozyme 100,000 units/ml and/or lysostafin 100 units/ml) per 1 slide glass, and thereafter, 1 ml of this enzyme reagent solution was dropped on a site of the leukocyte smear, and left to stand still for 30 minutes in a humid box at 37° C. to 42° C. Then, it was immersed in PBS containing 0.2 mol/l hydrochloric acid (prepared by adding hydrochloric acid to the PBS stock solution, 20 fold dilution in sterile purified water, and adjusting to give the final concentration of hydrochloric acid of 0.2 mol/l) and allowed infiltration on a shaker for 10 minutes as it was.

### Example 5

### Acetylation of Cell Membrane protein

Acetylation was carried out through immersing the slide glass in an acetylation reagent, which was prepared by adding acetic anhydride to an acetylating reagent (7.46 g of triethanolamine, an appropriate amount of hydrochloric acid, adjusted to give the total volume of 50 ml with an appropriate amount of sterile purified water) and diluting 10 fold in sterile purified water to give the final concentration of acetic anhydride of 0.8%, followed by shaking for 10 minutes on a shaker. Thereafter, the slide glass was sequentially immersed in 75%, 85%, and 98% ethanol for 3 minutes respectively, and completely air dried.

### Example 6

### Alkaline Treatment of DNA of Bacterial Body

### Denaturation from Double Strand to Single Strand

An alkaline treatment was carried out through immersing the slide glass in PBS which contains 70 mmol/l sodium hydroxide (prepared by adding sodium hydroxide in the PBS stock solution, diluting to 20 fold with sterile purified water to give the final concentration of sodium hydroxide of 70 mmol/l) for 3 minutes. Thereafter, the slide glass was sequentially immersed in 75%, 85%, and 98% ethanol for 3 minutes respectively, and completely air dried.

Example 7

### Hybridization

A solution containing 15 ng of a digoxigenin labelled DNA probe prepared with a probe dilution solution (including 0.25% SDS, 600 μl of salmon sperm DNA, 50 μl of 100× Denhardt's solution, 500 μl of a hybridization stock solution, 2250 μl of formamide, 1000 μl 50% dextran sulfate) is coated on the smeared site, and the slide was left to stand still in a humid box at 37° C. to 42° C. for 2 hours. A probe solution without including SDS was determined as a control. The digoxigenin labelled DNA probe was produced by a nick translation method. Thereafter, a hybridization washing solution (prepared by mixing a hybridization stock solution (13.15 g of sodium chloride, 6.615 g of trisodium citrate dihydrate, adjusted to give the total volume of 75 ml with sterile purified water) in a ratio of the hybridization stock solution:sterile purified water:formamide=5:4:50) was provided in three staining bottles, and sequentially the sample was immersed at 42° C. for 10 minutes, respectively.

Then, the sample was immersed in PBS, and shaken as it is on a shaker for 10 minutes. Digoxigenin labelled DNA probe utilized was each probe of SA-24 (SEQ ID NO: 1), SA-36 (SEQ ID NO: 2) and SA-77 (SEQ ID NO: 3), and SE-22 (SEQ ID NO: 4), SE-3 (SEQ ID NO: 5) and SE-32 (SEQ ID NO: 6) (see, Japanese Patent No. 2798499), as a probe for Staphylococcus aureus and Staphylococcus epidermidis. Further, as a probe for Pseudomonas aeruginosa, the probe of P2-2 (SEQ ID NO: 7) (see, Japanese Patent No. 2965544) was utilized. In addition, as probes for Enterococcus faecalis, EF-1 (SEQ ID NO: 8), EF-27 (SEQ ID NO: 9) and EF-7 (SEQ ID NO: 10) (see, Japanese Patent No. 2965543) were utilized. Additionally, as probes for Escherichia coli, Enterobacter cloacae and Klebsiella pneumoniae, EC-24 (SEQ ID NO: 11), EC-34 (SEQ ID NO: 12) and EC-39 (SEQ ID NO: 13), and ET-49 (SEQ ID NO: 14) and KI-50 (SEQ ID NO: 15) (see, Japanese Patent No. 3026789) were utilized. In addition, as probes for Candida albicans, CA-26 (SEQ ID NO: 16), CA-26-1 (SEQ ID NO: 17), CA-26-2 (SEQ ID NO: 18) and CA-26-3 (SEQ ID NO: 19) (see, Japanese Patent No. 2558420) were utilized. Using each sequence of these probes, each probe was produced by a nick translation method.

### Example 8

### Blocking

After carrying out in situ hybridization, an operation of blocking was performed. One ml of a blocking solution (2 ml of normal rabbit serum, 0.5 ml of the PBS stock solution, adjusted to give the total volume of 10 ml with sterile purified water) was dropped on the smear site per one slide glass in a humid box, and left to stand still for 30 minutes. Thereafter, the blocking reagent was removed.

### Example 9

### Reaction with Labelled Antibody

A labelled antibody solution was prepared by diluting a labelled antibody (1.05 unit of alkaline phosphatase labelled anti-digoxigenin antibody solution, adjusted with 12.6 μl of buffer A (746 mg of triethanolamine, 17.5 mg of sodium chloride, 20.3 mg of magnesium chloride hexahydrate, 1.36 mg of zinc chloride, 1000 mg of bovine serum albumine, an appropriate amount of hydrochloric acid, adjusted to give the total volume of 100 ml with sterile purified water) to give the total volume of 14 μl) in a labelled antibody diluent (8.48 mg of Tris-(hydroxymethyl)-aminomethane, 6.14 mg of sodium chloride, an appropriate amount of hydrochloric acid, adjusted to give the total volume of 0.7 ml with sterile purified water) to 50 fold, and each 10 μl of this labelled antibody solution was dropped on the smear site, followed by leaving to stand still for 30 minutes. Thereafter, it was immersed in a solution of a labelled antibody washing solution (1 ml of polysorbate 20, 50 ml of the PBS stock solution, adjusted to give the total volume of 100 ml with sterile purified water) diluted to 10 fold, and was allowed for infiltration on a shaker for 10 minutes as it was. After repeating this operation twice, it was immersed in a coloring pretreatment liquid obtained by mixing a coloring pretreatment liquid 1 (6.06 g of Tris-(hydroxymethyl)-aminomethane, 2.92 g of sodium chloride, an appropriate amount of hydrochloric acid, adjusted to give the total volume of 50 ml with sterile purified water) and a coloring pretreatment liquid 2 (5.08 g of magnesium chloride hexahydrate, adjusted to give the total volume of 50 ml with sterile purified water) in an equivalent volume and diluting to 5 fold with sterile purified water, and then shaken for 10 minutes on a shaker as it was.

### Example 10

### Detection

One ml of a coloring reagent (nitroblue tetrazolium (NBT)/5-bromo-4-chloro-3-indolylphosphate (BCIP) solution, pH 9.0 to 10.0:3.3 mg of NBT, 1.65 mg of BCIP, 99 μg of N,N-dimethylformamide, 121 mg of Tris-(hydroxymethyl)-aminomethane, an appropriate amount of hydrochloric acid, 58.4 mg of sodium chloride, 101.6 mg of magnesium chloride hexahydrate, adjusted to give the total volume of 10 ml with an appropriate amount of sterile purified water) per one slide glass was dropped on the smear site of the slide glass while filtration using a disposable syringe equipped with a 0.2 μm syringe top filter, and was left to stand still under light shielding in a humid box at 37° C. for 30 minutes. Thereafter, it was immersed in a solution of a coloring reagent washing solution (606 mg of Tris-(hydroxymethyl)-aminomethane, 186 mg of ethylenediamine tetraacetate disodium dihydrate, an appropriate amount of hydrochloric acid, adjusted to give the total volume of 50 ml with an appropriate amount of sterile purified water) diluted to 10 fold for 5 minutes, and was air dried. Then, it was immersed in a solution of a counter staining solution (50 mg of fast green FCF (edible dye, green color No. 3), adjusted to give the total volume of 50 ml with an appropriate amount of sterile purified water) diluted to 10 fold and then in 1% acetic acid solution. Thereafter, the excess counter staining solution was washed away by immersing again in a solution of the coloring reagent washing solution described above diluted to 10 fold followed by complete air drying.

### Example 11

### Determination

Determination was conducted by microscopic examination with a light microscope (×1,000), and observation of at least one color development of bluish purple color was determined as positive in cells within a single well stained with the counter staining solution. As a result, bacteria were detected in 5 specimens among 12 specimens by the process according to the present invention. Details of the 5 specimens were specimen A-SA (Staphylococcus aureus), specimens F and G-SE (Staphylococcus epidermidis), specimens J-SE and EF (Enterococcus faecalis), specimens L-SA and CA (Candida albicans). When blood culture was conducted using the same specimens according to a known method, SA was detected for the specimen A demonstrating the same result, however, any could not be detected for the specimens F, G, J and L. Therefore, it was revealed that the process of the present invention could achieve rapid detection with favorable sensitivity in comparison with blood culture.

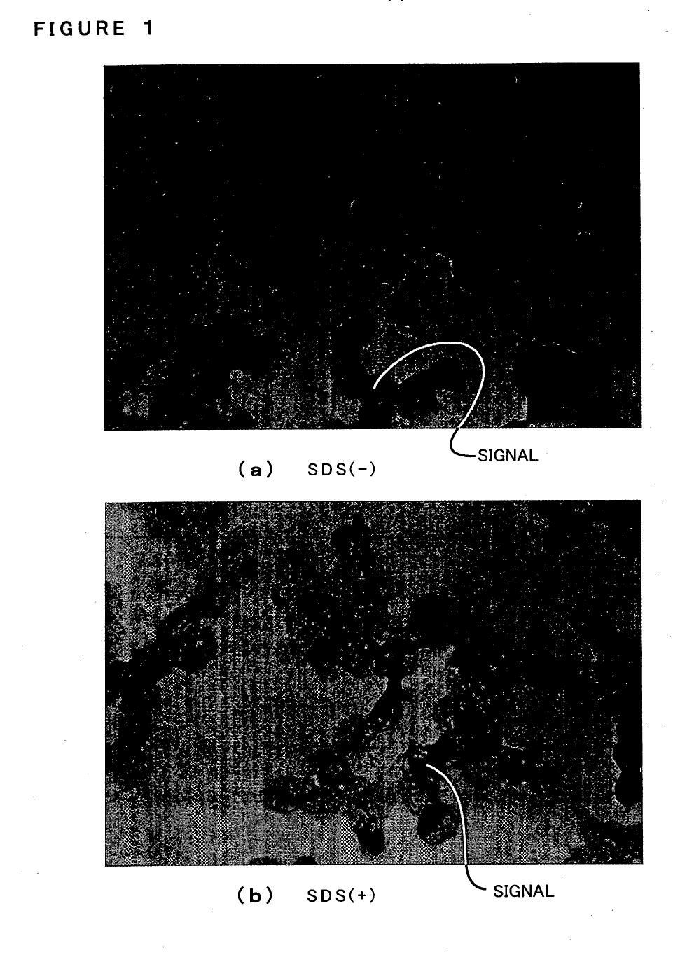

In connection with the results by the specimen A-SA, FIG. 1 illustrates the effects of addition of SDS to the probe dilution solution. It is clear that detection sensitivity of the signal can be markedly elevated by adding 0.25% SDS, as shown in FIG. 1. Also with respect to other specimens, detection of a favorable signal was similarly enabled by adding SDS. The probe used in this Example is a probe produced by nick translation using the base sequences of SA-24 (SEQ ID NO: 1), SA-36 (SEQ ID NO: 2) and SA-77 (SEQ ID NO: 3) in combination.

### Example 12

### Examination on Optimal Cell Number of Leukocytes to be Smeared and Fixed

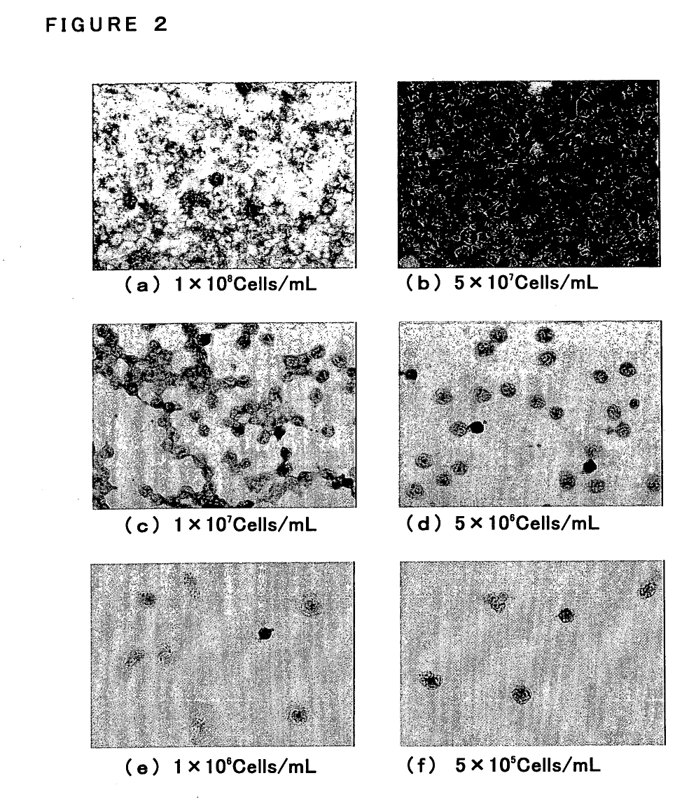

Optimal cell number of leukocytes to be smeared on the well of an APS coated slide glass (circular well having the diameter of 5 mm) was examined. Heparinized healthy human blood in an amount of 10 ml was collected, and leukocytes were obtained according to the procedure described in Example 1. Next, thus resulting leukocytes were suspended in an appropriate amount of PBS, and the cell number of the leukocytes per 1 ml was measured using a counting chamber. Starting from (a) 1×10⁸ cells/ml, a serial dilution of (b) 5×10⁷ cells/ml, (c) 1×10⁷ cells/ml, (d) 5×10⁶ cells/ml, (e) 1×10⁶ cells/ml, (f) 5×10⁵ cells/ml and (g) 1×10⁵ cells/ml was produced, and each 5 μl was smeared on the slide glass. After air drying, Carnoy fixation (see, Example 2) was carried out, and immediately stained with the aforementioned counter staining solution to execute the determination using the process described in Example 11. Consequently, cell number of 1×10⁸ cells/ml was excess, which was inadequate for the detection. Moreover, cell number of 5×10⁶ cells/ml or less results in small number of cells observed in the well, which was inadequate for the detection. Therefore, it is preferred that the density of the phagocytes to be immobilized (x cells/ml) is about 5×10⁶ cells/ml<x cells/ml <about 1×10⁸ cells/ml, and in particular, about 1×10⁷ cells/ml x cells/ml about 5×10⁷ cells/ml. In addition, corresponding thereto, it was revealed that cell number of the leukocytes fixed on the APS coated slide glass per 1 well (y cells/well (diameter of 5 mm)) may be prepared to be about 2.5×10⁴ cells/well<y cells/well (diameter of 5 mm)<about 5×10⁵ cells/well, and preferably, about 5×10⁴ cells/well y cells/well (diameter of 5 mm) about 2.5×10⁵ cells/well. Experimental results for the samples (a) to (f) are shown in FIGS. 2(a) to (f), respectively.

Example 13

### Selection of Lytic Enzyme for Use

Conditions for the enzyme to lyse Staphylococcus aureus (ATCC 12600), Staphylococcus epidermidis (ATCC 14990), Pseudomonas aeruginosa (ATCC 10145), Enterococcus faecalis (ATCC 19433) and Escherichia coli (ATCC 11775) were studied. For Staphylococcus aureus and Staphylococcus epidermidis, lysostafin was used as the lytic enzyme (Bur. J. Biochem., 38, 293-300, 1973). For Enterococcus faecalis, N-acetylmuramidase (Archs. Oral Biol., 23, 543-549, 1978) and lysozyme (Seikagaku Corporation) were used. Further, for Pseudomonas aeruginosa and Escherichia coli, PBS containing 70 mmol/l sodium hydroxide was used. Each type of these bacteria was inoculated in 5 ml of BHI (Brain Heart Infusion) liquid medium (manufactured by DIFCO), and cultured at 37° C. for 8 hours or longer. Thus cultured bacterial liquid was collected by centrifugation at 4° C. for 10 minutes at 2,000×g. The collected bacteria were suspended in PBS to give a sample.

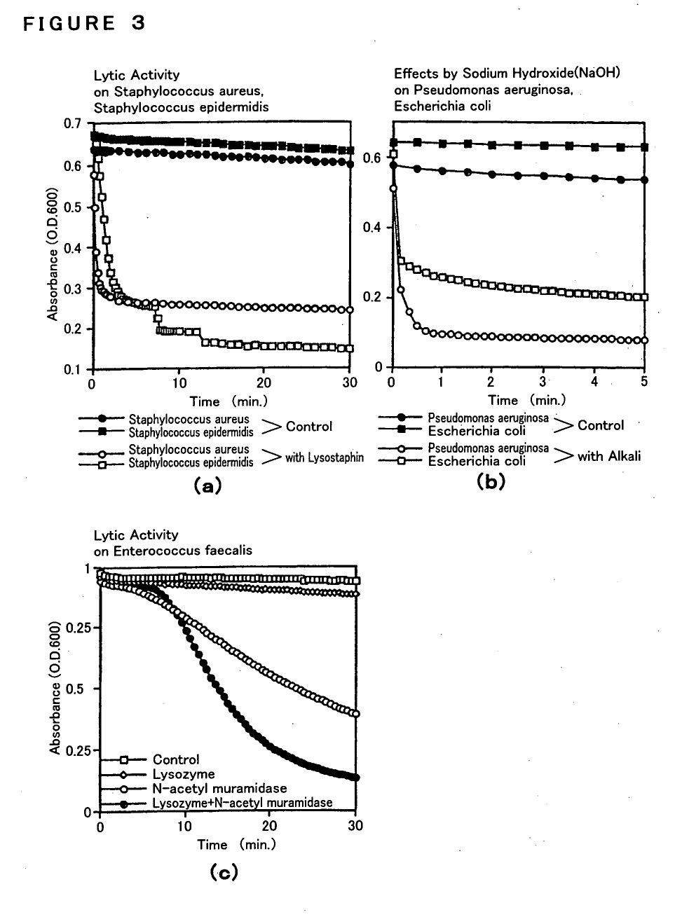

Lysis was evaluated by decrease in turbidity of the bacterial liquid at the absorbance of 600 nm using a microplate reader. Consequently, Staphylococcus aureus and Staphylococcus epidermidis were lysed by lysostafin. In respect of Pseudomonas aeruginosa and Escherichia coli, no enzymatic treatment was required because lysis was conducted with PBS containing 70 mmol/l sodium hydroxide. Furthermore, in connection with Enterococcus faecalis, it was proven that more excellent lytic activity could be achieved when lysozyme was used in combination than use of N-acetylmuramidase alone. Moreover, when the bacterium incorporated upon phagocytotic action is for example, Pseudomonas aeruginosa, Escherichia coli or the like, bacterial cell wall is lysed during the alkaline treatment to result in the state in which the gene is exposed. Therefore, it is not necessary to conduct this enzymatic treatment. Each enzyme for the pretreatment which is used in lysis of the foreign microorganism according to the present invention is effective not only for the aforementioned bacterial strain, but also for other bacterial strain that includes other genus staphylococcus, genus streptococcus, genus bacillus, genus micrococcus and the like. Additionally, each of such an enzyme can be used alone, but is more effective when used as a mixture. The results are illustrated in FIG. 3, specifically, in regard to: (a) Staphylococcus aureus and Staphylococcus epidermidis, (b) Pseudomonas aeruginosa and Escherichia coli, and (c) Enterococcus faecalis.

### Example 14

### Examination on Enzymatic Lysis Solution

### Examination on Optimal Concentration of DMSO

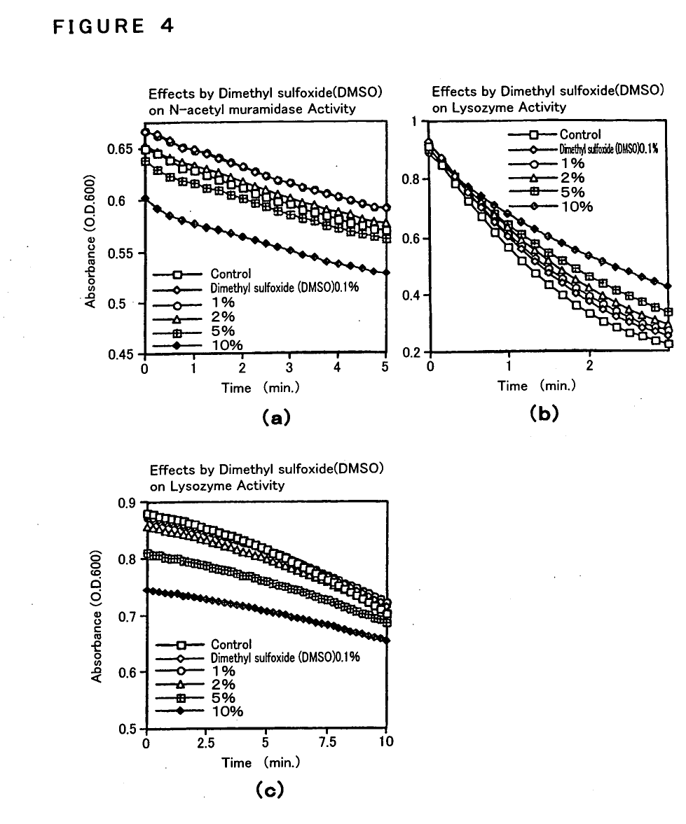

Because protease included in the enzyme reagent deteriorates the morphology of leukocytes, influences of DMSO, which is a solubilizer of PMSF added for the purpose of retaining the morphology of the leukocytes, on enzymatic activity were examined. Enterococcus faecalis was inoculated in 50 ml of the aforementioned BHI liquid medium, and cultured at 37° C. for 8 hours or longer. This culture liquid was centrifuged at 4° C. for 10 minutes at 2,000×g to collect the bacteria, followed by subjecting to a heat treatment in an autoclave (120° C., for 10 minutes) after suspending in PBS. Next, the suspension was centrifuged at 4° C. for 10 minutes at 2,000×g, and the supernatant was discarded. Precipitates were suspended in 1 ml of PBS, and thereafter, subjected to freeze-drying. This freeze-dried sample was suspended in 5 mmol/l Tris-hydrochloric acid (pH 6.0), 2 mmol/l magnesium chloride containing 0 to 10% DMSO to give the samples for N-acetylmuramidase. Further, Micrococcus luteus (JCM1464) was inoculated in 5 ml of BHI liquid medium (supra), and cultured at 37° C. for 8 hours or longer. The cultured bacterial liquid was centrifuged at 4° C. for 10 minutes at 2,000×g to collect the bacteria. After discarding the supernatant, and suspending and washing the bacterial pellet with 5 ml of PBS, centrifugation was conducted once again at 4° C. for 10 minutes at 2,000×g to collect the bacteria. Thus collected bacteria were suspended in PBS containing 0 to 10% DMSO to give the samples for lysozyme. On the other hand, Staphylococcus epidermidis was cultured and collected similarly to the instance of lysozyme, and suspended in PBS containing 0 to 10% DMSO to give the samples for lysostafin. Enzymatic activity was evaluated on the basis of the decrease in turbidity of the sample at the absorbance of 600 nm using a microplate reader. Each enzyme titer in this test was (a) N-acetylmuramidase: 300 unit/ml, (b) lysozyme: 10,000 unit/ml, (c) lysostafin: 50 unit/ml, and the influences of DMSO on the enzymatic activity were examined. As a result of evaluation of each enzymatic activity judging from the decrease per unit of time in turbidity (O.D.=600 nm) of the bacteria, DMSO hardly influenced on the N-acetylmuramidase activity. However, in respect of both lysozyme and lysostafin, decrease in activity was found with DMSO at the concentration of 5% or more. Additionally, no decrease in enzymatic activity was found with DMSO at the concentration of 2% or less. Accordingly, the concentration of DMSO for dissolving PMSF may be at least less than 5%, preferably 2% or less, more preferably approximately 1%. The results are show in FIG. 4(a) to (c) and Table 3 below.

TABLE 3
Influences of DMSO on Enzymatic Activity
(Decrease in turbidity of bacteria)

| Amount of | | | |
|---|---|---|---|
| added | N-acetylmuramidase | lysozyme | Lysostafin |
| DMSO (%) | O.D./5 minutes | O.D./3 minutes | O.D./10 minutes |
| 0 | 79.3 ± 4.8 | 0.689 ± 0.028 | 0.168 ± 0.017 |
| (control) | | | |
| 0.1 | 75.0 ± 3.2 | 0.678 ± 0.026 | 0.164 ± 0.009 |
| 1 | 75.8 ± 2.8 | 0.660 ± 0.026 | 0.160 ± 0.008 |
| 2 | 75.8 ± 2.5 | 0.653 ± 0.024 | 0.145 ± 0.009 |
| 5 | 76.3 ± 4.9 | 0.566 ± 0.017 | 0.124 ± 0.006 |
| 10 | 73.8 ± 3.5 | 0.464 ± 0.016 | 0.094 ± 0.006 |

### Example 15

### Examination on Enzymatic Lysis Solution

### Examination on Optimal Concentration of PMSF

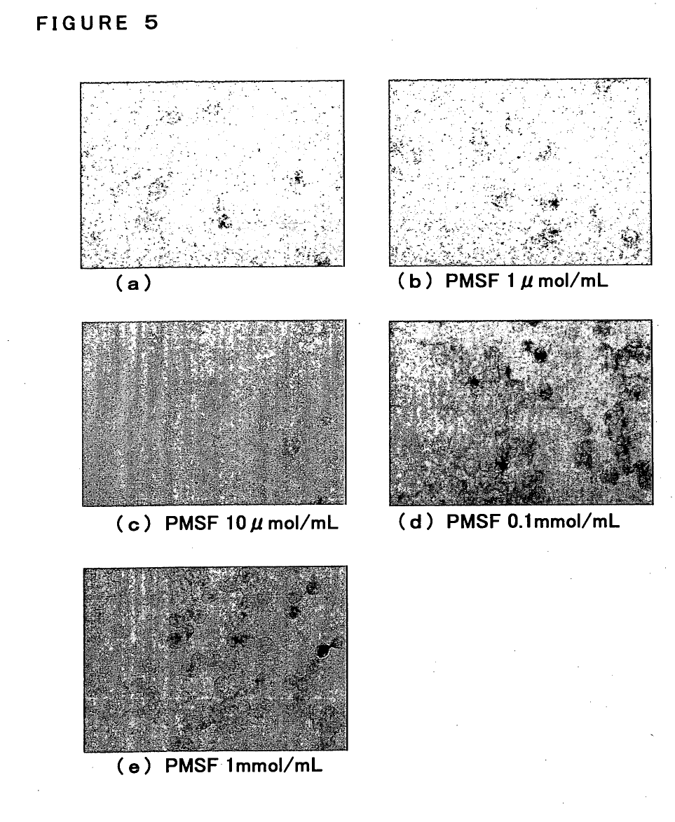

Because protease included in the enzyme reagent deteriorates the morphology of leukocytes, effects of PMSF (manufactured by PIERCE), which is added for the purpose of retaining the morphology of the leukocytes, on enzymatic activity were examined. PMSF was dissolved in 100 μl of DMSO (manufactured by Wako Pure Chemical Industries, Ltd), and diluted to 10 ml with PBS such that the final concentration of PMSF becomes none (0 mmol/l) to 1 mmol/l. To this solution was added proteinase K (manufactured by Boeringer Mannheim) such that titer of the protease becomes 0.2 unit/ml. Heparinized healthy human blood in an amount of 5 ml was collected, and leukocytes were obtained according to the process described in Example 1. Next, the leukocytes were suspended in an appropriate amount of PBS, and the cell number was measured using a counting chamber. Cell number was adjusted to about 5×10⁴ cells/well to about 2.5×10⁵ cells/well, and 5 μl therefrom was smeared on the well of the APS coated slide glass. After air drying, fixation was executed according to the method of Carnoy fixation described in Example 2. Using this sample, tests were performed according to the process described in Examples 3 to 11. As a consequence of performing the tests at the concentration of PMSF of 1 μmol/l to 1 mmol/l, effects were found at the concentration of 10 μmol/l or greater, while deterioration of morphology of the leukocytes was completely suppressed at the concentration of PMSF of 0.1 mmol/l or greater. The results are shown in FIG. 5, for (a): protease 0.2 unit/ml alone, (b): 1 μmol/ml PMSF added, (c): 10 82 mol/ml PMSF added, (d): 0.1 mmol/ml PMSF added; and (e): 1 mmol/ml PMSF added, respectively.

### Example 16

### Examination of Optimal Titer of Lytic Enzyme, Zymolase

Optimal titer of zymolase for exposing DNA was examined through lysis of Candida albicans. Candida albicans was inoculated in YPD medium, and cultured over day and night at 30° C. Then, two types of the solutions were prepared: a solution of Candida albicans as a substrate suspended in PBS (substrate 1); and a solution prepared by Carnoy s fixation, immersing in 70% ethanol, air drying and suspension in PBS (substrate 2). Upon the reaction, a mixture of zymolase/PBS: 0.5 ml, substrate: 1.5 ml, M/15 phosphate buffer: 2.5 ml and sterile purified water: 0.5 ml, adjusted to give the total volume of 5.0 ml was used.

Thereafter, the reaction was allowed at 37° C. for 2 hours, and the OD₈₀₀ was measured. Furthermore, the concentration of zymolase (Zymolyase-100T) for use was 0 mg/ml, 0.01 mg/ml, 0.025 mg/ml, 0.05 mg/ml, 0.1 mg/ml, 0.25 mg/ml, 0.5 mg/ml, 1 mg/ml, 2.5 mg/ml and 5 mg/ml. Consequently, each OD₈₀₀ value when the substrate 1 was used was 0.533, 0.521, 0.553, 0.554, 0.548, 0.417, 0.394, 0.288, 0.163 and 0.113, and each OD₈₀₀ value when the substrate 2 was used was 0.445, 0.411,0.359, 0.282, 0.232, 0.146, 0.115, 0.096, 0.08 and 0.057. It was proven that effectiveness was brought when both of the substrate 1 and substrate 2 were in the range of 0.5 mg/ml to 5 mg/ml, and particularly 1 mg/ml to 5 mg/ml. That is, the amount of zymolase to be used is preferably 50 unit/ml to 500 unit/ml, particularly 100 unit/ml to 500 unit/ml.

### Example 17

### Examination of Optimal Condition (Titer) of Enzymatic Treatment

(1) Production of Digested Sample

[1] Preparation of U937 Cell

U937 cells (monocyte established cell line: ATCC CRL-1593.2) were cultured in an RPMI 1640 medium (25 ml) within a cell culture flask (175 cm²)in a 5% carbon dioxide gas incubator at 37° C. Next, the U937 cell culture liquid was placed in a 50 ml centrifuge tube, and centrifuged at 4° C. for 10 minutes at 220×g to recover the U937 cells. Then, thus recovered U937 cells were suspended in 200 μl of PBS, and the cell number was counted with a counting chamber. The cell number was adjusted to 1×10⁴ cells/μl to 2×10⁴ cells/μl.

[2] Preparation of Bacterial Digested Sample

Staphylococcus aureus (ATCC 12600), Staphylococcus epidermidis (ATCC 14990), Pseudomonas aeruginosa (ATCC 10145), Enterococcus faecalis (ATCC 19433) and Escherichia coli (ATCC 11775) were inoculated in each 5 ml of BHI culture medium, and cultured at 37° C. for 8 hours or longer. The cultured bacterial liquid was centrifuged at 4° C. for 10 minutes at 2,000×g to collect the bacteria. After discarding the supernatant, the bacterial pellet was suspended in 5 ml of PBS, and centrifugation was conducted once again at 4° C. for 10 minutes at 2,000×g to collect the bacteria. Thus collected bacteria were suspended in 5 ml of PBS and thereafter, 15 ml of bacterial liquids was produced prepared by diluting in PBS to give the turbidity (O.D.=600 nm) of the bacterial liquid, which was measured with a absorbance meter, of 0.01 to 0.03 for Staphylococcus aureus, 0.01 to 0.03 for Staphylococcus epidermidis, 0.02 to 0.03 for Pseudomonas aeruginosa, 0.01 to 0.03 for Enterococcus faecalis, 0.02 to 0.03 for Escherichia coli, respectively. Thus produced bacterial liquid was transferred into a separate 175 cm² flask for culture, and left to stand still at room temperature for 30 minutes. Fifty ml of heparinized healthy human blood was collected, and thereto was added the reagent for separating hemocyte at a ratio of 4:1, and left to stand still at 37° C. for 30 minutes to yield the leukocyte fraction. Thus obtained leukocyte fraction was adjusted to 50 ml with PBS. The supernatant in the culture flask (supra) was gently discarded, and each 10 ml of the leukocyte fraction diluted in PBS was added to the flask followed by leaving to stand still at room temperature for 10 minutes. The supernatant in the flask for culture was discarded, and the leukocytes attached to the bottom of the flask were recovered in a 15 ml centrifuge tube with 10 ml of PBS containing 0.02% EDTA, and centrifuged at 4° C. for 10 minutes at 140×g to 180×g to collect the leukocytes. Because contamination of erythrocytes was found in the collected leukocytes, precipitates of the leukocytes were gently suspended in 1 ml of sterile purified water to allow hemolysis, subjected to isotonization through adding 14 ml of PBS, followed by centrifugation once again at 4° C. for 10 minutes at 140×g to 180×g to collect the leukocytes. The collected leukocytes were suspended in PBS, and cell number was counted with a counting chamber to adjust to give 1×10⁴ cells/μl to 5×10⁴ cells/μl. These digested samples were referred to as SA digested sample, SE digested sample, PA digested sample, EF digested sample and EK digested sample.

[3] Smear and Fixation

Each 5 μl of U937 cells prepared in Example 17 (1) [1] and each 5 μl of each bacterial digested sample produced in Example 17 (1) [2] were smeared on each well of the APS coated slide glass, and air dried. Next, after immersing the slide glass in the Carnoy s fixative described in Example 2 for 20 minutes, it was immersed in 75% ethanol for 5 minutes. After washing Carnoy's fixative and air drying, the slide glass was stored at 4° C. until use in the test (see, Example 2). Next, pretreatment of the fixed sample was carried out according to Example 3.

(2) Standard and Process for Testing Digested Sample

[1] Cell Number

Cell number to be smeared and fixed on the slide glass of each bacterial digested sample was 5.0×10⁴ to 2.5×10⁵ cells/well, whilst cell number of U937 cells was 5.0×10⁴ to 1.0×10⁵ cells/well.

[2] Phagocytosis Rate

The bacterial digested sample smeared and fixed on the slide glass was stained with an acridine orange staining solution, and about 200 cells were randomly counted with a fluorescence microscope (×1,000). Among the measured cells, cells including bacteria phagocytized within the cells (cells with any change characteristics in phagocytosis found in morphology, as shown by arrows in FIG. 6) were determined as positive cells, and the phagocytosis rate (%) was calculated according to the mathematical formula below.
Phagocytosis rate (%)=[(Positive cell number/Measured cell number)×100]

Thus calculated phagocytosis rate (%) of each bacterial digested sample was 10% or greater.

[3] Test Process

The digested sample produced in Example 17 (2) [1] and [2] was employed as a specimen. The SA digested sample used had the phagocytosis rate of 23% with 1.98×10⁵ cells/well. The SE digested sample had the phagocytosis rate of 27% with 1.74×10⁵ cells/well. Moreover, the EF digested sample had the phagocytosis rate of 34% with 6.40×10⁴ cells/well.

Using the slide glass having each digested sample smeared thereon, the enzymatic pretreatment was performed according to the process described in Example 3. Next, the slide glass after completing the enzymatic pretreatment was placed in a humid box, and the reaction was allowed by dropping 1 ml of each enzyme solution prepared to give each titer on the smeared site of the specimen. Thereafter, the slide glass was immersed in PBS containing 0.2 mol/l hydrochloric acid, and in 70% ethanol respectively, for 10 minutes, and air dried. After immersing this slide glass in PBS containing 70 mmol/l sodium hydroxide for 3 minutes, and in 70% ethanol for 10 minutes, it was air dried and stained with 1% acridine orange staining solution. Then, evaluation was made with a fluorescence microscope (×1,000). For Staphylococcus aureus and Staphylococcus epidermidis, examination of the optimal titer was conducted with lysostafin. In order to examine the optimal titer when N-acetylmuramidase and lysozyme are used in combination for Enterococcus faecalis, examination on optimal titer of lysozyme was conducted in cases where N-acetylmuramidase was fixed at 100 unit/ml, and on optimal titer of N-acetylmuramidase in cases where lysozyme was fixed at 10,000 unit/ml. The determination was made as adequate when no bacterial body was identified in the leukocytes by the enzymatic treatment.

[4] Results

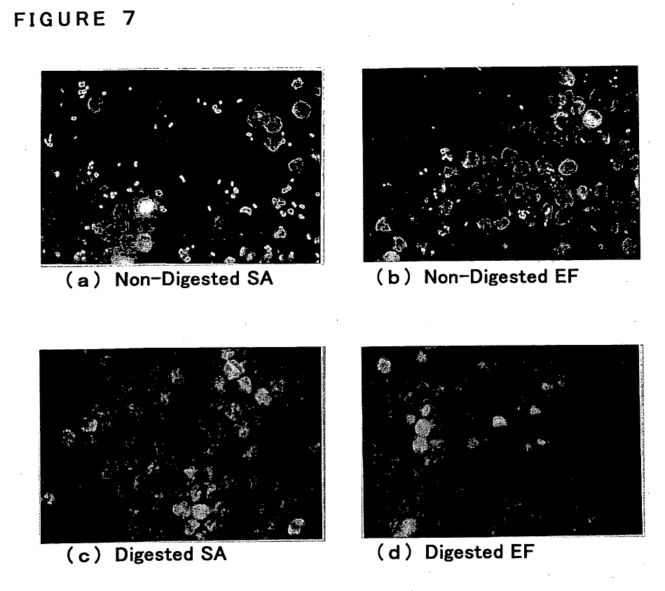

For the lysis of Staphylococcus aureus, as described in Table 4, sufficient effects were exerted at the titer of lysostafin of 1 unit/ml, however, upon lysis of Staphylococcus epidermidis, the titer of lysostafin of 10 unit/ml or greater was necessary. Therefore, the optimal titer of lysostafin was set to be 10 unit/ml to 100 unit/ml. In addition, for the lysis of Enterococcus faecalis, lysis did not occur with the titer of N-acetylmuramidase of 10 unit/ml or less when the titer of lysozyme was fixed at 10,000 unit/ml. In respect of lysozyme, when the titer of N-acetylmuramidase was fixed at 100 unit/ml, lysis did not occur with the titer of lysozyme of 1,000 unit/ml or less, as described in Table 5. Therefore, the optimal titer of N-acetylmuramidase was set to be 100 unit/ml to 1,000 unit/ml, whilst the optimal titer of lysozyme was set to be 10,000 unit/ml to 100,000 unit/ml. The results are shown in FIG. 7. In the Figure, depicted are states of: (a) the digested sample of Staphylococcus aureus prior to the enzymatic treatment, (b) the digested sample of Enterococcus faecalis prior to the enzymatic treatment, (c) the sample (a) following the enzymatic treatment, and (d) the sample (b) following the enzymatic treatment.

TABLE 4
Optimal Titer for Enzymatic Treatment
of Lysostafin on S. Aureus, S. epidermidis

| (U/mL) | | | | | | | |
|---|---|---|---|---|---|---|---|
| Digested | | Lysostafin Titer | | | | | |
| Samples | | 0 | 0.1 | 1 | 10 | 100 | 1,000 |
| SA | once | inadequate | Inadequate | adequate | adequate | adequate | adequate |
| Digested | twice | inadequate | inadequate | adequate | adequate | adequate | adequate |
| Sample | thrice | inadequate | inadequate | adequate | adequate | adequate | adequate |
| SE | once | inadequate | inadequate | inadequate | adequate | adequate | adequate |
| Digested | twice | inadequate | inadequate | inadequate | adequate | adequate | adequate |
| Sample | thrice | inadequate | inadequate | inadequate | adequate | adequate | adequate |

TABLE 5
Optimal Titer of Enzymatic Treatment
of N-acetylmuramidase and lysozyme on E. faecalis

| titer (U/mL) | | N-acetylmuramidase | | | | | |
|---|---|---|---|---|---|---|---|
| Digested Sample | | 0 | 1 | 10 | 100 | 1,000 | 10,000 |
| EF | once | Inadequate | inadequate | inadequate | adequate | adequate | adequate |
| Digested | twice | Inadequate | inadequate | inadequate | adequate | adequate | adequate |
| Sample | thrice | Inadequate | inadequate | inadequate | adequate | adequate | adequate |
| (U/mL) | | | | | | | |
| Digested | | Lysozyme titer | | | | | |
| Sample | | 0 | 10 | 100 | 1,000 | 10,000 | 100,000 |
| EF | once | Inadequate | inadequate | inadequate | inadequate | adequate | adequate |
| Digested | twice | Inadequate | inadequate | inadequate | inadequate | adequate | adequate |
| Sample | thrice | Inadequate | inadequate | inadequate | inadequate | adequate | adequate |

Applications of these results obtained using the digested samples to the present invention could result in similar results. Therefore, the optimal titer of each enzyme as described above in the identification of a causative microorganism of an infectious disease in the clinical specimen of the present invention was also set similarly.

### Example 18

### Examination on Optimal Condition of Enzymatic Treatment (Temperature)

Using a slide glass including each digested sample smeared thereon, examination was conducted according to the process described in example 17 (2) [3]. Time period of the enzymatic treatment in this test was 30 minutes, and the temperature for examination was 4° C., 25° C., 37° C., 42° C., and 60° C. Moreover, titer of each enzyme was N-acetylmuramidase (100 unit/ml, manufactured by Seikagaku Corporation), lysozyme (10,000 unit/ml, manufactured by Seikagaku Corporation), lysostafin (10 unit/ml, manufactured by SIGMA).

Determination was conducted according to the process described in example 17 (2) [3]. As a consequence, for Staphylococcus aureus, no bacterial body was found in the leukocytes in the range of temperature of 4° C. to 60° C. For Staphylococcus epidermidis, although bacterial bodies remained in the leukocytes at the temperature of 4° C. and 25° C., no bacterial body was found at 37° C. or higher. Further, for Enterococcus faecalis, although bacterial bodies remained at the temperature of treatment of 4° C., 25° C. and 60° C., no bacterial body was found at 37° C. and 42° C. Hence, the optimal temperature for the enzymatic treatment was set to be 37° C. to 42° C. The results are shown in Table 6.

TABLE 6
Optimal Temperature for Treatment of Enzyme Reagent

| | | Temperature for | | | | |
|---|---|---|---|---|---|---|
| Digested | | Treatment (° C.) | | | | |
| Samples | | 4 | 25 | 37 | 42 | 60 |
| SA | Once | ade- | adequate | adequate | adequate | adequate |
| Di- | | quate | | | | |
| gested | twice | ade- | adequate | adequate | adequate | adequate |
| Sample | | quate | | | | |
| | thrice | ade- | adequate | adequate | adequate | adequate |
| | | quate | | | | |
| SE | once | inade- | inadequate | adequate | adequate | adequate |
| Di- | | quate | | | | |
| gested | twice | inade- | inadequate | adequate | adequate | adequate |
| Sample | | quate | | | | |
| | thrice | inade- | inadequate | adequate | adequate | adequate |
| | | quate | | | | |
| EF | once | inade- | inadequate | adequate | adequate | inadequate |
| Di- | | quate | | | | |
| gested | twice | inade- | inadequate | adequate | adequate | inadequate |
| Sample | | quate | | | | |
| | thrice | inade- | inadequate | adequate | adequate | inadequate |
| | | quate | | | | |

Applications of these results obtained using the digested samples to the present invention could result in similar results. Therefore, the optimal temperature of the enzymatic treatment in the identification of a causative microorganism of an infectious disease in the clinical specimen of the present invention was also set similarly.

### Example 19

### Examination on Optimal Condition of Enzymatic Treatment (Time)

Digested samples produced according to the process described in Example 17 (1) [1] and [2] were used as specimens. Time period of the examination was 0 minute, 10 minutes, 20 minutes, 30 minutes, 60 minutes and 120 minutes. Phagocytosis rate of the used SA digested sample was 18% with 7.80×10⁴ cells/well. Phagocytosis rate of the used SE digested sample was 34% with 1.10×10⁵ cells/well. Further, phagocytosis rate of the EF digested sample was 28% with 1.30×10⁵ cells/well.

Using the slide glass including each digested sample smeared thereon, examination was conducted according to the process described in example 17 (2) [3]. Temperature for the enzymatic treatment in this test was 37° C., and titer of each enzyme was 100 unit/ml for N-acetylmuramidase, 10,000 unit/ml for lysozyme, 10 unit/ml for lysostafin. Determination was conducted according to the process described in example 17 (2) [3]. As a consequence, for all of Staphylococcus aureus, Staphylococcus epidermidis and Enterococcus faecalis digested samples, no bacterial body was found in the leukocytes with the time period of the enzymatic treatment of 20 minutes or longer (inadequate at 0 minute and 10 minutes). Therefore, the optimal time period of the enzymatic treatment is at least 15 minutes or longer, preferably 20 minutes or longer, and still preferably 30 minutes to 60 minutes. The results are shown in Table 7.

TABLE 7
Optimal Time Period of Treatment of Enzyme Reagent

| | | Time of | | | | | |
|---|---|---|---|---|---|---|---|
| | | enzyme-treatment | | | | | |
| Digested | | (minutes) | | | | | |
| Samples | | 0 | 10 | 20 | 30 | 60 | 120 |
| SA | once | inadequate | inadequate | adequate | adequate | adequate | adequate |
| Digested | twice | inadequate | inadequate | adequate | adequate | adequate | adequate |
| Sample | thrice | inadequate | inadequate | adequate | adequate | adequate | adequate |
| SE | once | inadequate | inadequate | adequate | adequate | adequate | adequate |
| Digested | twice | inadequate | inadequate | adequate | adequate | adequate | adequate |
| Sample | thrice | inadequate | inadequate | adequate | adequate | adequate | adequate |
| EF | once | inadequate | inadequate | adequate | adequate | adequate | adequate |
| Digested | twice | inadequate | inadequate | adequate | adequate | adequate | adequate |
| Sample | thrice | inadequate | inadequate | adequate | adequate | adequate | adequate |

Applications of these results obtained using the digested samples to the present invention could result in similar results. Therefore, the optimal time period of the enzymatic treatment in the identification of a causative microorganism of an infectious disease in the clinical specimen of the present invention was also set similarly.

### Example 20

### Examination on Optimal Condition of Enzymatic Treatment (Time)

In in situ hybridization reaction according to the present invention, concentration of the probe is an important factor which affects the hybridizing velocity. When the probe concentration is too low, the reaction velocity may be lowered, leading to the possibility of unclear signal. Furthermore, use of an excess amount of probe may result in grounds for nonspecific reaction.

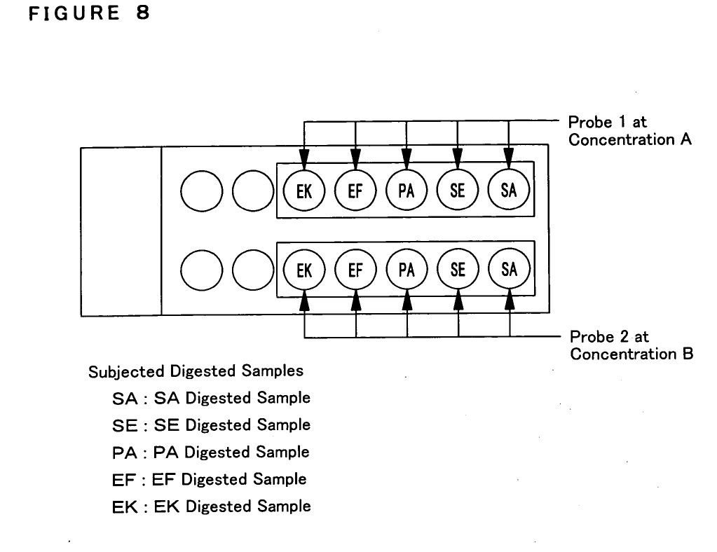

Thus, optimal concentration was examined in connection with various probe solutions. First, the digested samples produced according to the process described in Example 17(1) [1] and [2] were used as specimens. The phagocytosis rate of the used SA digested sample was 24% with 1.48×10⁵ cells/well. The phagocytosis rate of the SE digested sample was 28% with 2.07×10⁵ cells/well. The phagocytosis rate of the PA digested sample was 11% with 1.59×10⁵ cells/well. In addition, the phagocytosis rate of the EF digested sample was 24% with 1.72×10⁵ cells/well. The phagocytosis rate of the EK digested sample was 12% with 1.63×10⁵ cells/well. Using the slide glass including each digested sample smeared thereon, examination was conducted according to the process described in Example 17(2) [3]. The probes for use were labelled with digoxigenin, and the concentration of each probe for Staphylococcus aureus, Staphylococcus epidermidis, Enterococcus faecalis, Pseudomonas aeruginosa and Escherichia coli was adjusted to 0.06 ng/μl, 0.6 ng/μl, 1.2 ng/μl, 1.8 ng/μl, 2.4 ng/μl, 3 ng/μl, respectively. The probe solution prepared to each concentration described above was used on the slide glass including the digested sample smeared thereon (see, FIG. 8), and examined according to the process described in Examples 3-11.

Consequently, the signal became unclear at lower concentration (0.06 ng/μl), and on the other hand, increase in background was observed at higher concentration (2.4 ng/μl and 3 ng/μl). Therefore, the concentrations of probes of SA, SE, PA, EF and EK were determined to be 0.6 to 1.8 ng/μl, preferably 0.6 to 1.2 ng/μl. Moreover, since an inadequate result was yielded at 0.06 ng/μl, while an adequate result was yielded at 0.6 ng/μl, it is preferably determined to be 0.1 ng/μl or greater.

Furthermore, since an inadequate result was yielded at 2.4 ng/μl, and an adequate result was yielded at 1.8 ng/μl, it is preferably determined to be 2.2 ng/μl or less. The results are shown in Tables 8-12 below.

TABLE 8
SA probe

Probe concentration
(ng/μL)

| Digested sample | 0.06 | 0.6 | 1.2 | 1.8 | 2.4 | 3 |
|---|---|---|---|---|---|---|
| SA digested sample | − | + | + | + | + | + |
| SE digested sample | − | − | − | − | + | + |
| PA digested sample | − | − | − | − | + | + |
| EF digested sample | − | − | − | − | + | + |
| EK digested sample | − | − | − | − | + | + |

TABLE 9
SE probe

Probe concentration
(ng/μL)

| Digested sample | 0.06 | 0.6 | 1.2 | 1.8 | 2.4 | 3 |
|---|---|---|---|---|---|---|
| SA digested sample | − | − | − | − | − | + |
| SE digested sample | − | + | + | + | + | + |
| PA digested sample | − | − | − | − | − | + |
| EF digested sample | − | − | − | − | − | + |
| EK digested sample | − | − | − | − | − | + |

TABLE 10
PA probe

Probe concentration
(ng/μL)

| Digested sample | 0.06 | 0.6 | 1.2 | 1.8 | 2.4 | 3 |
|---|---|---|---|---|---|---|
| SA digested sample | − | − | − | − | − | − |
| SE digested sample | − | − | − | − | + | + |
| PA digested sample | − | + | + | + | + | + |
| EF digested sample | − | − | − | − | − | + |
| EK digested sample | − | − | − | − | − | + |

TABLE 11
EF probe

Probe concentration
(ng/μL)

| Digested sample | 0.06 | 0.6 | 1.2 | 1.8 | 2.4 | 3 |
|---|---|---|---|---|---|---|
| SA digested sample | − | − | − | − | − | + |
| SE digested sample | − | − | − | − | + | + |
| PA digested sample | − | − | − | − | + | + |
| EF digested sample | − | + | + | + | + | + |
| EK digested sample | − | − | − | − | − | − |

TABLE 12
EK probe

Probe concentration
(ng/μL)

| Digested sample | 0.06 | 0.6 | 1.2 | 1.8 | 2.4 | 3 |
|---|---|---|---|---|---|---|
| SA digested sample | − | − | − | − | + | + |
| SE digested sample | − | − | − | − | + | + |
| PA digested sample | − | − | − | − | + | + |
| EF digested sample | − | − | − | − | + | + |
| EK digested sample | − | + | + | + | + | + |

Applications of these results obtained using the digested samples to the present invention could result in similar results. Therefore, the optimal concentration of each probe described above in the identification of a causative microorganism of an infectious disease in the clinical specimen of the present invention was also set similarly.

### Example 21

### Examination on Hybridization Temperature

Reaction temperature in the hybridization reaction is a parameter which affects the hybridizing velocity and stability of the hybrid. Because high temperature of the hybridization reaction is known to deteriorate the cell morphology, examination of the optimal temperature (4° C., 25° C., 37° C., 42° C., 50° C. and 60° C.) was performed.

First, the digested samples produced according to the process described in Example 17(1) [1] and [2] were used as specimens. The phagocytosis rate of the used SA digested sample was 31% with 1.38×10⁵ cells/well. The phagocytosis rate of the SE digested sample was 42% with 1.95×10⁵ cells/well. The phagocytosis rate of the PA digested sample was 14% with 1.27×10⁵ cells/well. In addition, the phagocytosis rate of the EF digested sample was 48% with 1.05×10⁵ cells/well. The phagocytosis rate of the EK digested sample was 17% with 1.85×10⁵ cells/well.

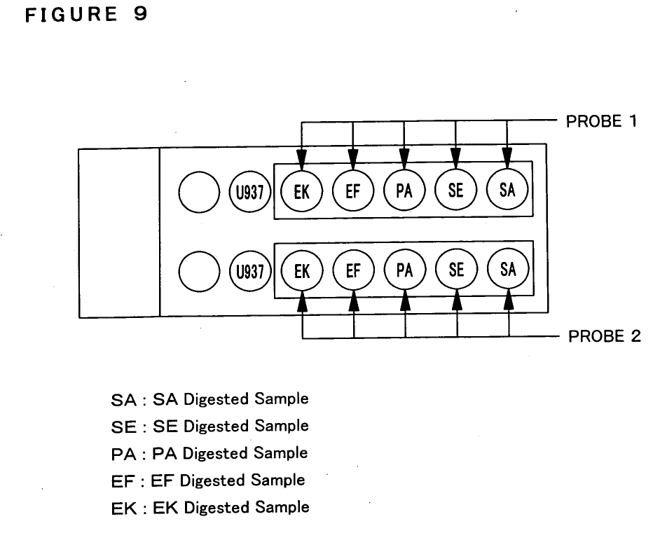

Using the slide glass including the digested sample and U937 cells smeared and fixed thereon (see, FIG. 9), examination was conducted according to the process described in Examples 3-11. Consequently, no stable signal was observed for each type of probe at the hybridization temperature of 4° C. or less owing to the lowered hybridization velocity. Further, at 60° C., changes in cell morphology were detected, and thus no stable signal was observed. In addition, at 25° C. and 50° C., detection could be executed better compared to at the temperature of 37° C. and 42° C., although the signal was unclear. Hence, optimal temperature of the hybridization may be 25° C. to 50° C., more preferably 37 to 42° C. The results are shown in Tables 13-17 below.

TABLE 13
SA probe

Hybridization
temperature (° C.)

| Digested sample | 4 | 25 | 37 | 42 | 50 | 60 |
|---|---|---|---|---|---|---|
| SA digested sample | − | + | + | + | + | + |
| SE digested sample | − | − | − | − | − | − |
| PA digested sample | − | − | − | − | − | − |
| EF digested sample | − | − | − | − | − | − |
| EK digested sample | − | − | − | − | − | − |

TABLE 14
SE probe

Hybridization temperature (° C.)

| Digested sample | 4 | 25 | 37 | 42 | 50 | 60 |
|---|---|---|---|---|---|---|
| SA digested sample | − | − | − | − | − | − |
| SE digested sample | + | + | + | + | + | − |
| PA digested sample | − | − | − | − | − | − |
| EF digested sample | − | − | − | − | − | − |
| EK digested sample | − | − | − | − | − | − |

TABLE 15
PA probe

Hybridization temperature (° C.)

| Digested sample | 4 | 25 | 37 | 42 | 50 | 60 |
|---|---|---|---|---|---|---|
| SA digested sample | − | − | − | − | − | − |
| SE digested sample | − | − | − | − | − | − |
| PA digested sample | − | + | + | + | + | − |
| EF digested sample | − | − | − | − | − | − |
| EK digested sample | − | − | − | − | − | − |

TABLE 16
EF probe

| | Hybridization temperature (° C.) | | | | | |
|---|---|---|---|---|---|---|
| Digested sample | 4 | 25 | 37 | 42 | 50 | 60 |
| SA digested sample | − | − | − | − | − | − |
| SE digested sample | − | − | − | − | − | − |
| PA digested sample | − | − | − | − | − | − |
| EF digested sample | + | + | + | + | + | − |
| EK digested sample | − | − | − | − | − | − |

TABLE 17
EK probe

Hybridization temperature (° C.)

| Digested sample | 4 | 25 | 37 | 42 | 50 | 60 |
|---|---|---|---|---|---|---|
| SA digested sample | − | − | − | − | − | − |
| SE digested sample | − | − | − | − | − | − |
| PA digested sample | − | − | − | − | − | − |
| EF digested sample | − | − | − | − | − | − |
| EK digested sample | − | + | + | + | + | − |

Applications of these results obtained using the digested samples to the present invention could result in similar results. Therefore, the optimal temperature of hybridization in the identification of a causative microorganism of an infectious disease in the clinical specimen of the present invention was also set similarly.

### Example 22

### Examination on Hybridization Time Period

The digested samples produced according to the process described in Example 17(1) [1] and [2] were used as specimens, and examination was conducted on the time period of hybridization of 10 minutes, 60 minutes, 90 minutes, 120 minutes, 180 minutes and 900 minutes. The phagocytosis rate of the used SA digested sample was 47% with 1.45×10⁵ cells/well. The phagocytosis rate of the SE digested sample was 47% with 1.33×10⁵ cells/well. The phagocytosis rate of the PA digested sample was 15% with 1.91×10⁵ cells/well. In addition, the phagocytosis rate of the EF digested sample was 41% with 1.45×10⁵ cells/well. The phagocytosis rate of the EK digested sample was 20% with 1.23×10⁵ cells/well.

Using the slide glass including the digested sample and U937 cells smeared and fixed thereon (same as one shown in FIG. 9), examination was conducted according to the process described in Examples 3-11. Consequently, although no signal was observed with the time period of hybridization of 10 minutes, a signal was observed at 60 minutes or greater, and a stable signal was observed at 90 minutes or greater. Further, no alteration in detection of the signal was observed also with the time period of hybridization of 900 minutes. Therefore, it is preferred that the time period is at least 30 minutes or greater, preferably 60 minutes or greater, and more preferably 90 minutes or greater. More preferred optimal time period of hybridization may be set to be 120 minutes to 900 minutes. The results are shown in Tables 18-22 below.

TABLE 18
SA probe

| | Hybridization time (minutes) | | | | | |
|---|---|---|---|---|---|---|
| Digested sample | 10 | 60 | 90 | 120 | 180 | 900 |
| SA digested sample | − | + | + | + | + | + |
| SE digested sample | − | − | − | − | − | − |
| PA digested sample | − | − | − | − | − | − |
| EF digested sample | − | − | − | − | − | − |
| EK digested sample | − | − | − | − | − | − |

TABLE 19
SE probe

| | Hybridization time (minutes) | | | | | |
|---|---|---|---|---|---|---|
| Digested sample | 10 | 60 | 90 | 120 | 180 | 900 |
| SA digested sample | − | − | − | − | − | − |
| SE digested sample | + | + | + | + | + | + |
| PA digested sample | − | − | − | − | − | − |
| EF digested sample | − | − | − | − | − | − |
| EK digested sample | − | − | − | − | − | − |

TABLE 20
SE probe

| | Hybridization time (minutes) | | | | | |
|---|---|---|---|---|---|---|
| Digested sample | 10 | 60 | 90 | 120 | 180 | 900 |
| SA digested sample | − | − | − | − | − | − |
| SE digested sample | − | − | − | − | − | − |
| PA digested sample | − | + | + | + | + | + |
| EF digested sample | − | − | − | − | − | − |
| EK digested sample | − | − | − | − | − | − |

TABLE 21
EF probe

| | Hybridization time (minutes) | | | | | |
|---|---|---|---|---|---|---|
| Digested sample | 10 | 60 | 90 | 120 | 180 | 900 |
| SA digested sample | − | − | − | − | − | − |
| SE digested sample | − | − | − | − | − | − |
| PA digested sample | − | − | − | − | − | − |
| EF digested sample | + | + | + | + | + | + |
| EK digested sample | − | − | − | − | − | − |

TABLE 22
EK probe

| | Hybridization time (minutes) | | | | | |
|---|---|---|---|---|---|---|
| Digested sample | 10 | 60 | 90 | 120 | 180 | 900 |
| SA digested sample | − | − | − | − | − | − |
| SE digested sample | − | − | − | − | − | − |
| PA digested sample | − | − | − | − | − | − |
| EF digested sample | − | − | − | − | − | − |
| EK digested sample | − | + | + | + | + | + |

Applications of these results obtained using the digested samples to the present invention could result in similar results. Therefore, the optimal time period of hybridization in the identification of a causative microorganism of an infectious disease in the clinical specimen of the present invention was also set similarly.

### Example 23

### Influence of Surfactant Added to Hybridization Solution

The digested samples produced according to the process described in Example 17(1) [1] and [2] were used as specimens. When any of various surfactants (SDS, lauryl sarcosine, saponin, BRIJ35, Tween 20, Triton X-100) was added to the probe dilution solution followed by hybridization carried out according to Example 7, the detection sensitivity was dramatically enhanced by adding 0.25% SDS. In addition, the detection sensitivity could be improved by lauryl sarcosine, BRIJ 35 or Tween 20. The results are shown in Table 23 below.

TABLE 23

| Surfactant | Signal detection |
|---|---|
| None added | + |
| SDS | +++ |
| Lauryl sarcosine | ++ |
| Saponin | + |
| BRIJ 35 | ++ |
| Tween 20 | ++ |
| Triton X-100 | + |

Furthermore, as a consequence of using SDS at various concentrations, it was revealed that preferable concentration was 1% or less, more preferably 0.1% to 0.5%, and still more preferably 0.25%.

Applications of these results obtained using the digested samples to the present invention could result in similar results. Therefore, also in the present invention, it is preferred that a surfactant, particularly SDS, is added at the step of in situ hybridization.

### Example 24

### Examination on Chain Length of Probe Used in Hybridization

Staphylococcus aureus probe (SA-24 (SEQ ID NO: 1)) and Pseudomonas aeruginosa probe (P2-2 (SEQ ID NO: 7)) were labelled with digoxigenin.

First, 1 μg of purified each type of the DNA probe was prepared to give the total volume of 50 μg with 5 μl of 10×L.B. (0.5 mol/l Tris-hydrochloric acid (pH 7.5), 50 mmol/l magnesium chloride, 5 μl of 0.5 mg bovine serum albumin), 5 μl of 100 mmol/l dithiothreitol, each 1 nmol of dNTPs (A, G, C), 0.5 nmol of digoxigenin-dUTP (Dig-dUTP), each 0.5 nmol of dTTP, 3 μl of DNase (amount corresponding to 25 mU, 75 mU and 200 mU), 1 μl of 10 U/μl DNA polymerase and an appropriate amount of sterile purified water. Digoxigenin labelling was performed at 15° C. for 2 hours. After the labelling, the mixture was boiled for 5 minutes to terminate the reaction. The reaction liquid after the termination was injected into a spin column (CENTRI-SEP COLUMUNS CS901, PRINCETON SEPARATIONS, INC.), and centrifuged at 25° C. for 2 minutes (3,000×g) to remove free nucleotides. Then, concentration of the eluate was measured by an absorbance meter. Electrophoresis was performed on a 3% agarose gel to confirm the size.

Next, DNA in the agarose gel was transferred to a nitrocellulose membrane by Southern blotting method. Then, the membrane was immersed in 2% blocking reagent (manufactured by Roche) for 30 minutes, and thereafter, alkaline phosphatase labelled anti-digoxigenin antibody in an amount of 1/5,000 was added thereto and the immersion was allowed for 30 minutes. Next, the membrane was washed twice by shaking in 100 mmol/l Tris-hydrochloric acid (pH 7.5) and 150 mmol/l sodium chloride for 10 minutes. Thereafter, washing was executed by shaking in 100 mmol/l Tris-hydrochloric acid (pH9.5) and 150 mmol/l sodium chloride for 10 minutes. Then, color development was conducted by immersing in an NBT/BCIP solution.

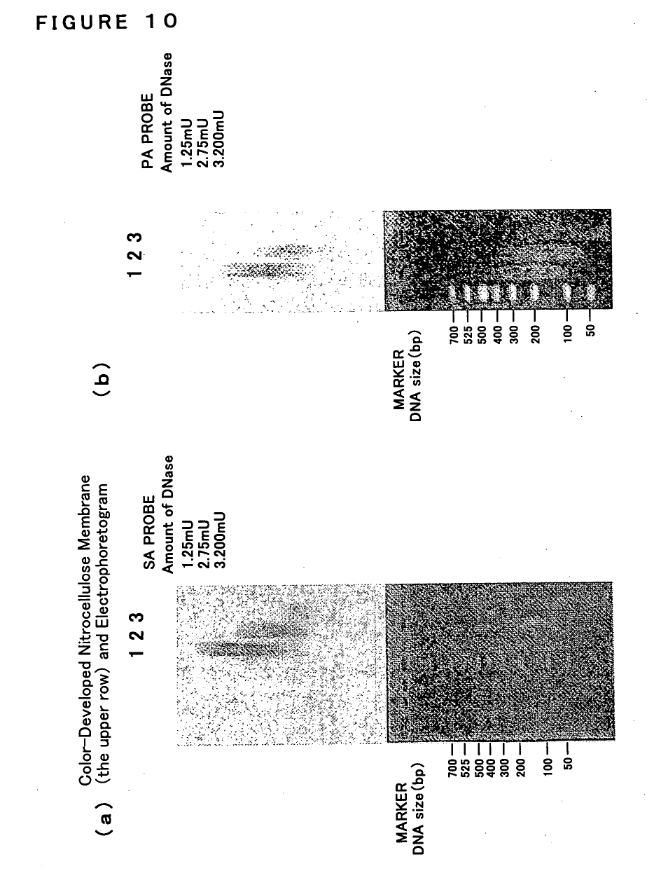

Finally, the membrane was immersed in purified water to stop the color development, and dried. Consequently, as shown in FIG. 10 for (a) use of the SA probe and (b) use of the PA probe, respectively, it was indicates that in cases where cleavage was conducted using 25 mU of DNase (in Figure, lane 1) such that the chain length distributes the base length of predominantly about 350 to about 600, high labelling efficiency was achieved. When thus resulting probe for detection was used in the process for detecting a causative microorganism of an infectious disease according to the present invention in which a digested sample or a clinical specimen from a patient suffering from an infectious disease was used to carry out hybridization, a signal could be detected with excellent sensitivity. Therefore, it was reveled that chain length of the probe used in the hybridization may be the base length of about 350 to about 600, and preferably the base length of about 350 to about 550.

### Example 25

### Examination on Probe used in Hybridization

Escherichia coli digested samples produced according to the process described in Example 17(1) [1] and [2] were used as specimens to examine on the probes for detection.

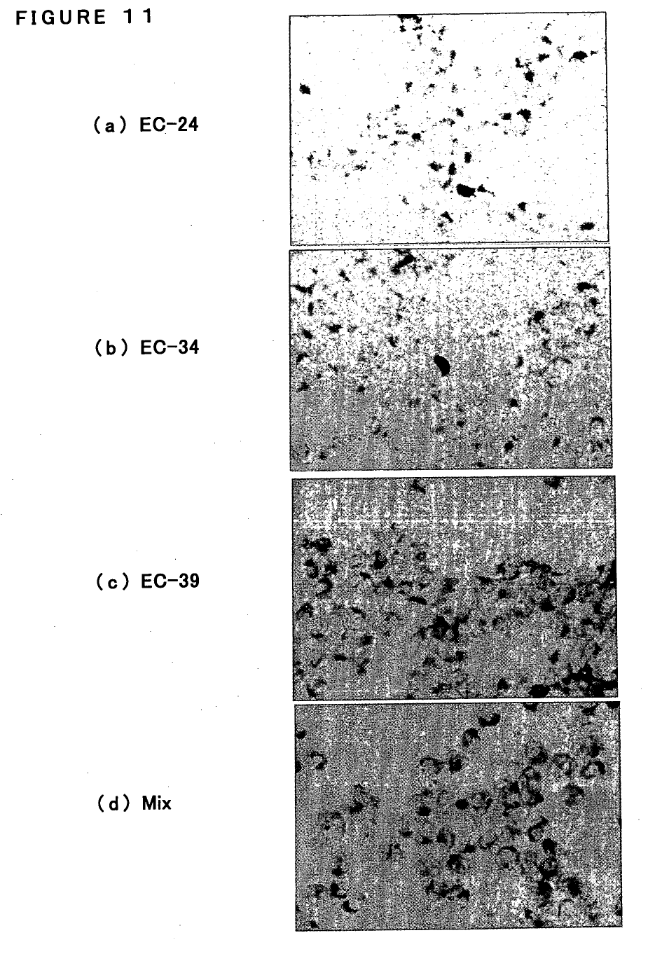

Probes for detection were prepared through labelling with digoxigenin as described in Example 24 from EC-24 (SEQ ID NO: 11), EC-34 (SEQ ID NO: 12) and EC-39 (SEQ ID NO: 13) such that they have the base length of about 350 to about 600, and used alone or in combination of those three, respectively. From thus obtained results, it was evident that the signal could be detected more clearly resulting in elevated sensitivity for (d) the mixed probe MIX prepared by mixing the three ((a) EC-24, (b) EC-34 and (c) EC-39), than for each (a) EC-24, (b) EC-34 or (c) EC-39 used alone, as shown in FIG. 11.

### Example 26

### Comparison of Detection Capability for Various Amount of Bacteria Between Blood Culture Process and Process for Detecting Foreign Microorganisms Wherein Digested Sample of Present Invention is Used

S. aureus, S. epidermidis or Enterococcus faecalis described in Example 13 was admixed with healthy human blood at the concentration of 10⁵, 10⁴, 10³, 10², 10¹ or 10⁰ CFU/ml. After incubating the mixture, tests were performed with a kit for identifying a causative microorganism of an infectious disease of a clinical specimen (Hybrizep [trade name: FUSO PHARMACEUTICAL INDUSTRIES, LTD.]), and by a blood culture process according to any known process. Furthermore, after bringing each bacterium contact with piperacillin (PIPC) having a broad spectrum at a concentration of 10 MIC, each bacterium was admixed with healthy human blood at the concentration of 10⁴, 10³, 10², 10¹ or 10⁰ CFU/ml, and the tests were similarly performed. The results are shown in Table 24 to Table 26 below.

TABLE 24

| | | Staphylococcus aureus | | | | | |
|---|---|---|---|---|---|---|---|
| | | Bacterial | | | | | |
| | | concentration (CFU/mL) | | | | | |
| | | 10⁵ | 10⁴ | 10³ | 10² | 10¹ | 10⁰ |
| PIPC | Present process | + | + | + | + | + | − |
| untreated | Blood culture | + | + | + | + | + | − |
| PIPC | Present process | d | + | + | + | − | − |
| treated | Blood culture | d | + | + | − | − | − |

d: not performed

TABLE 25

| | | Staphylococcus epidermidis | | | | | |
|---|---|---|---|---|---|---|---|
| | | Bacterial | | | | | |
| | | concentration (CFU/mL) | | | | | |
| | | 10⁵ | 10⁴ | 10³ | 10² | 10¹ | 10⁰ |
| PIPC | Present process | + | + | + | + | − | − |
| untreated | Blood culture | + | + | + | + | + | − |
| PIPC | Present process | d | + | + | + | − | − |
| treated | Blood culture | d | − | − | − | − | − |

d: not performed

TABLE 26

| Enterococcus faecalis | | | | | |
|---|---|---|---|---|---|
| Bacterial | | | | | |
| concentration (CFU/mL) | | | | | |
| 10⁵ | 10⁴ | 10³ | 10² | 10¹ | 10⁰ |

| | | | | | | | |
|---|---|---|---|---|---|---|---|
| PIPC | Present process | + | + | + | + | − | − |
| untreated | Blood culture | + | + | + | + | + | − |
| PIPC | Present process | d | + | + | + | − | − |
| treated | Blood culture | d | + | + | − | − | − |

d: not performed

Apparently from the results described above, in the tests performed using the bacteria subjected to a treatment with an antibiotic, the bacteria could not be detected in the blood culture process even in an amount of bacteria with which the detection was enabled in instances where the treatment with the antibiotic was not conducted, however the bacteria could be detected without being affected by the antibiotic through the use of Hybrizep.

### Example 27

### Determination of Sensitivity Test

Among the performance tests according to the present invention, availability of the digested sample in the sensitivity tests was examined. Each operation process herein was conducted according to the procedure described in Examples 2-11.

Digested samples used were SA digested sample, SE digested sample, PA digested sample, EF digested sample and EK digested sample. Each digested sample was produced by the process described in Example 17. SA digested sample had the phagocytosis rate of 29% and cell number of 1.05×10⁵ cells/well, and was determined as adequate. The SE digested sample had the phagocytosis rate of 47% and cell number of 1.51×10⁵ cells/well, and was determined as adequate PA digested sample had the phagocytosis rate of 19% and cell number of 1.99×10⁵ cells/well, and was determined as adequate. EF digested sample had the phagocytosis rate of 33% and cell number of 1.25×10⁵ cells/well, and was determined as adequate. EK digested sample had the phagocytosis rate of 19% and cell number of 1.13×10⁵ cells/well, and was determined as adequate

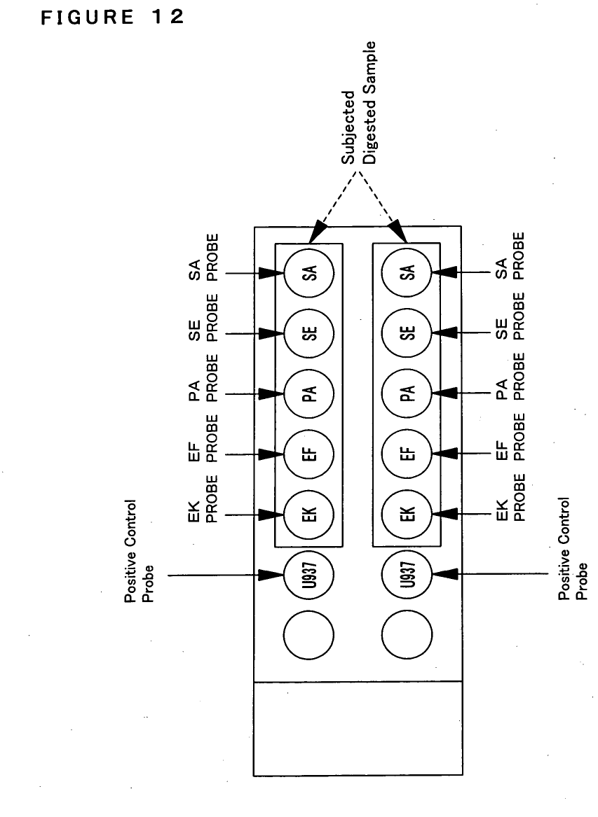

As shown in FIG. 12, tests were performed according to the process described in Examples 2-3, using the slide glass including the digested sample smeared thereon.

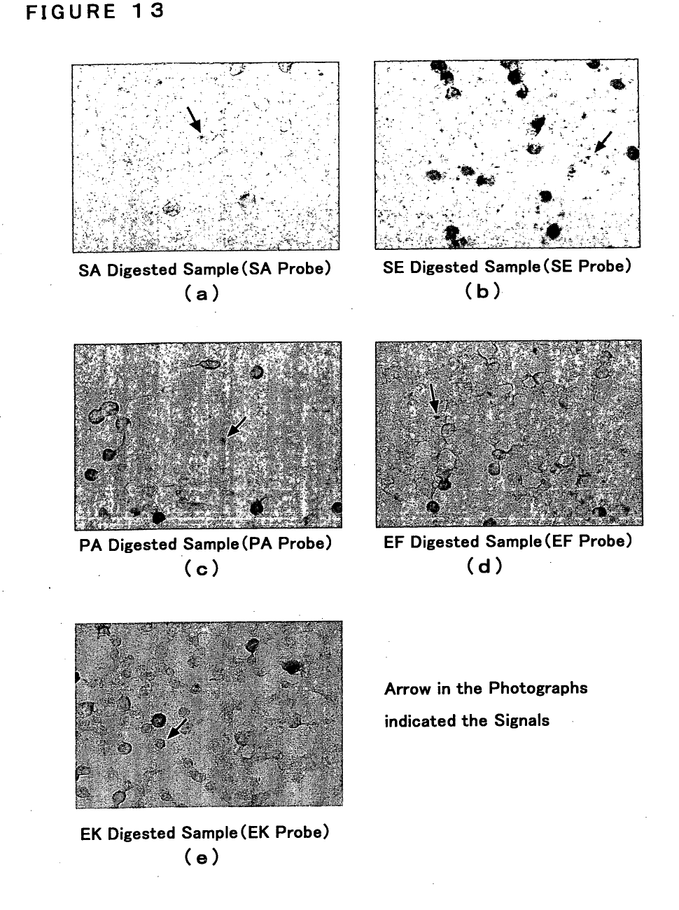

Each of SA, SE, PA, EF and EK digested samples was subjected to tests of three times per single kit, and the tests were performed for 3 kits. Thus, as shown in Table 27 and FIG. 13(a) to (e), the bacteria could be detected in all of the digested samples. Hence, it was proven that the digested samples were useful in the sensitivity test in the process demonstrated in Examples 1-11. Therefore, standard of the sensitivity test process of demonstrated in Examples 1-11 was defined as one which enables detection of a signal when the test was performed according the procedure described in Examples 2-11 using the digested sample of a known bacterium.

TABLE 27

| | Trial | | | |
|---|---|---|---|---|
| Digested Samples | time(s) | Kit 1 | Kit 2 | Kit 3 |
| SA Digested Sample | 1 | SA detected | SA detected | SA detected |
| | 2 | SA detected | SA detected | SA detected |
| | 3 | SA detected | SA detected | SA detected |
| SE Digested Sample | 1 | SE detected | SE detected | SE detected |
| | 2 | SE detected | SE detected | SE detected |
| | 3 | SE detected | SE detected | SE detected |
| PA Digested Sample | 1 | PA detected | PA detected | PA detected |
| | 2 | PA detected | PA detected | PA detected |
| | 3 | PA detected | PA detected | PA detected |
| EF Digested Sample | 1 | EF detected | EF detected | EF detected |
| | 2 | EF detected | EF detected | EF detected |
| | 3 | EF detected | EF detected | EF detected |
| EK Digested | 1 | EK detected | EK detected | EK detected |
| Sample | 2 | EK detected | EK detected | EK detected |
| | 3 | EK detected | EK detected | EK detected |

### Example 28

### Determination of Specificity Test

Among the performance tests, availability of the digested sample in the specificity tests was examined. Each operation process herein was conducted according to the procedure described in Examples 2-11.

Digested samples used were SA digested sample, SE digested sample, PA digested sample, EF digested sample and EK digested sample. Each digested sample was produced by the process described in Example 17. SA digested sample had the phagocytosis rate of 29% and cell number of 1.05×10⁵ cells/well, and was determined as adequate. The SE digested sample had the phagocytosis rate of 47% and cell number of 1.51×10⁵ cells/well, and was determined as adequate. PA digested sample had the phagocytosis rate of 19% and cell number of 1.99×10⁵ cells/well, and was determined as adequate. EF digested sample had the phagocytosis rate of 33% and cell number of 1.25×10⁵ cells/well, and was determined as adequate. EK digested sample had the phagocytosis rate of 19% and cell number of 1.13×10⁵ cells/well, and was determined as adequate

As shown in FIG. 12, tests were performed according to the procedure described in Examples 2-3, using the slide glass including the digested sample smeared thereon.

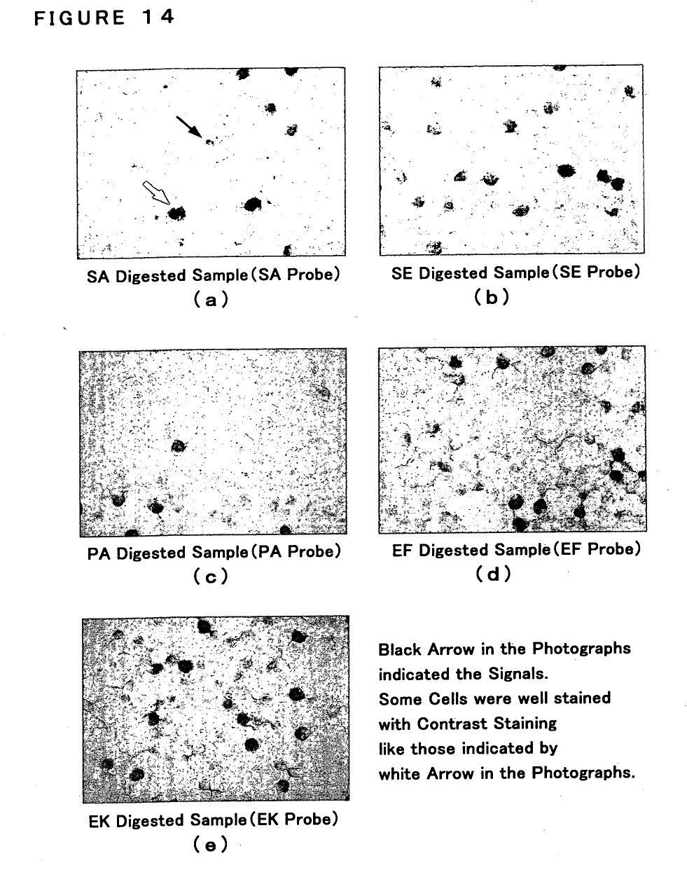

Each of SA, SE, PA, EF and EK digested samples was subjected to tests of three times per one probe included in single kit, and the tests were performed for 3 kits. Thus, as shown in Tables 28-31, an accurate signal could be detected for any of all the known bacterial digested samples. FIG. 14(a) to (e) illustrates that SA digested sample can be specifically detected by only the probe (a) for detecting SA. Hence, it was proven that the digested samples were useful in the specificity test of the process demonstrated in Examples 1-11. Therefore, standard of the specificity test of the process demonstrated in Examples 1-11 was defined as one which enables detection of a signal for only the corresponding bacterial digested sample when the test was performed according the procedure described in Examples 2-11 using the digested sample of a known bacterium.

TABLE 28

| | Type of probe | | | | | | | | | | | | | | |
|---|---|---|---|---|---|---|---|---|---|---|---|---|---|---|---|
| Kit 1 | SA | | | SE | | | PA | | | EF | | | EK | | |
| Digested Samples | 1 | 2 | 3 | 1 | 2 | 3 | 1 | 2 | 3 | 1 | 2 | 3 | 1 | 2 | 3 |
| SA Digested Sample | + | + | + | − | − | − | − | − | − | − | − | − | − | − | − |
| SE Digested Sample | − | − | − | + | + | + | − | − | − | − | − | − | − | − | − |
| PA Digested Sample | − | − | − | − | − | − | + | + | + | − | − | − | − | − | − |
| EF Digested Sample | − | − | − | − | − | − | − | − | − | + | + | + | − | − | − |
| EK Digested Sample | − | − | − | − | − | − | − | − | − | − | − | − | + | + | + |

TABLE 29

| | Type of probe | | | | | | | | | | | | | | |
|---|---|---|---|---|---|---|---|---|---|---|---|---|---|---|---|
| Kit 2 | SA | | | SE | | | PA | | | EF | | | EK | | |
| Digested Samples | 1 | 2 | 3 | 1 | 2 | 3 | 1 | 2 | 3 | 1 | 2 | 3 | 1 | 2 | 3 |
| SA Digested Sample | + | + | + | − | − | − | − | − | − | − | − | − | − | − | − |
| SE Digested Sample | − | − | − | + | + | + | − | − | − | − | − | − | − | − | − |
| PA Digested Sample | − | − | − | − | − | − | + | + | + | − | − | − | − | − | − |
| EF Digested Sample | − | − | − | − | − | − | − | − | − | + | + | + | − | − | − |
| EK Digested Sample | − | − | − | − | − | − | − | − | − | − | − | − | + | + | + |

TABLE 30

| | Type of probe | | | | | | | | | | | | | | |
|---|---|---|---|---|---|---|---|---|---|---|---|---|---|---|---|
| Kit 3 | SA | | | SE | | | PA | | | EF | | | EK | | |
| Digested Samples | 1 | 2 | 3 | 1 | 2 | 3 | 1 | 2 | 3 | 1 | 2 | 3 | 1 | 2 | 3 |
| SA Digested Sample | + | + | + | − | − | − | − | − | − | − | − | − | − | − | − |
| SE Digested Sample | − | − | − | + | + | + | − | − | − | − | − | − | − | − | − |
| PA Digested Sample | − | − | − | − | − | − | + | + | + | − | − | − | − | − | − |
| EF Digested Sample | − | − | − | − | − | − | − | − | − | + | + | + | − | − | − |
| EK Digested Sample | − | − | − | − | − | − | − | − | − | − | − | − | + | + | + |

TABLE 31

| Kit | | Positive Control Probe | | | Negative Control Probe | | |
|---|---|---|---|---|---|---|---|
| 1 | Sample | 1 | 2 | 3 | 1 | 2 | 3 |
| | U937 cell | + | + | + | − | − | − |
| 2 | Sample | 1 | 2 | 3 | 1 | 2 | 3 |
| | U937 cell | + | + | + | − | − | − |
| 3 | Sample | 1 | 2 | 3 | 1 | 2 | 3 |
| | U937 cell | + | + | + | − | − | − |

### Example 29

### Determination of Reproducibility Test

Availability of the digested sample in the reproducibility tests was examined.

Each operation process herein was conducted according to the procedure described in Examples 2-11.

Digested samples used were SA digested sample, SE digested sample, PA digested sample, EF digested sample and EK digested sample. Each digested sample was produced by the process described in Example 17. SA digested sample had the phagocytosis rate of 29% and cell number of 1.05×10⁵ cells/well, and was determined as adequate. SE digested sample had the phagocytosis rate of 47% and cell number of 1.5×10⁵ cells/well, and was determined as adequate. PA digested sample had the phagocytosis rate of 19% and cell number of 1.99×10⁵ cells/well, and was determined as adequate. EF digested sample had the phagocytosis rate of 33% and cell number of 1.25×10⁵ cells/well, and was determined as adequate. EK digested sample had the phagocytosis rate of 19% and cell number of 1.13×10⁵ cells/well, and was determined as adequate.

As shown in FIG. 12, tests were performed according to the procedure described in Examples 2-3, using the slide glass including the digested sample smeared thereon.

Each of SA, SE, PA, EF and EK digested samples was subjected to tests of three times per one probe included in single kit, and the tests were performed for 3 kits. Thus, as shown in Tables 32-35, an accurate signal could be detected for any of all the bacterial digested samples. Hence, it was proven that the digested samples were useful in applying the reproducibility test of the process demonstrated in Examples 1-11. Therefore, standard of the reproducibility test of the process demonstrated in Examples 1-11 was determined to comply with Examples 2-11 using a digested sample of a known bacterium, and was defined as one which leads the identical effects when the specificity tests are repeated three times at the same time.

TABLE 32

| | Type of probe | | | | | | | | | | | | | | |
|---|---|---|---|---|---|---|---|---|---|---|---|---|---|---|---|
| Kit 1 | SA | | | SE | | | PA | | | EF | | | EK | | |
| Digested Samples | 1 | 2 | 3 | 1 | 2 | 3 | 1 | 2 | 3 | 1 | 2 | 3 | 1 | 2 | 3 |
| SA Digested Sample | + | + | + | − | − | − | − | − | − | − | − | − | − | − | − |
| SE Digested Sample | − | − | − | + | + | + | − | − | − | − | − | − | − | − | − |
| PA Digested Sample | − | − | − | − | − | − | + | + | + | − | − | − | − | − | − |
| EF Digested Sample | − | − | − | − | − | − | − | − | − | + | + | + | − | − | − |
| EK Digested Sample | − | − | − | − | − | − | − | − | − | − | − | − | + | + | + |

TABLE 33

| | Type of probe | | | | | | | | | | | | | | |
|---|---|---|---|---|---|---|---|---|---|---|---|---|---|---|---|
| Kit 2 | SA | | | SE | | | PA | | | EF | | | EK | | |
| Digested Samples | 1 | 2 | 3 | 1 | 2 | 3 | 1 | 2 | 3 | 1 | 2 | 3 | 1 | 2 | 3 |
| SA Digested Sample | + | + | + | − | − | − | − | − | − | − | − | − | − | − | − |
| SE Digested Sample | − | − | − | + | + | + | − | − | − | − | − | − | − | − | − |
| PA Digested Sample | − | − | − | − | − | − | + | + | + | − | − | − | − | − | − |
| EF Digested Sample | − | − | − | − | − | − | − | − | − | + | + | + | − | − | − |
| EK Digested Sample | − | − | − | − | − | − | − | − | − | − | − | − | + | + | + |

TABLE 34

| | Type of probe | | | | | | | | | | | | | | |
|---|---|---|---|---|---|---|---|---|---|---|---|---|---|---|---|
| Kit 2 | SA | | | SE | | | PA | | | EF | | | EK | | |
| Digested Samples | 1 | 2 | 3 | 1 | 2 | 3 | 1 | 2 | 3 | 1 | 2 | 3 | 1 | 2 | 3 |
| SA Digested Sample | + | + | + | − | − | − | − | − | − | − | − | − | − | − | − |
| SE Digested Sample | − | − | − | + | + | + | − | − | − | − | − | − | − | − | − |
| PA Digested Sample | − | − | − | − | − | − | + | + | + | − | − | − | − | − | − |
| EF Digested Sample | − | − | − | − | − | − | − | − | − | + | + | + | − | − | − |
| EK Digested Sample | − | − | − | − | − | − | − | − | − | − | − | − | + | + | + |

TABLE 35

| Kit | | Positive Cntrol Probe | | | Negative Control Probe | | |
|---|---|---|---|---|---|---|---|
| 1 | Sample | 1 | 2 | 3 | 1 | 2 | 3 |
| | U937 cell | + | + | + | − | − | − |
| 2 | Sample | 1 | 2 | 3 | 1 | 2 | 3 |
| | U937 cell | + | + | + | − | − | − |
| 3 | Sample | 1 | 2 | 3 | 1 | 2 | 3 |
| | U937 cell | + | + | + | − | − | − |

### INDUSTRIAL APPLICABILITY

According to the present invention, a phagocytotic function of a phagocyte can be evaluated in vitro, and an experimental model is stably provided which can be utilized for a variety of objects such as evaluation of immune functions, evaluation of efficiency of differentiation of a phagocyte, screening of a modulator of phagocytotic functions, determination of effects, guidelines for dosage regimen in clinical tests, markers for the diagnosis of infectious diseases, performance tests of kit and the like.

1. A digested phagocyte prepared by contacting in vitro a phagocyte with a foreign microorganism and isolating the phagocyte so contacted.

2. The digested phagocyte according to claim 1 wherein a turbidity of bacterial liquid (O.D.=600 nm) of the foreign microorganism used for in vitro contact between the phagocyte and the foreign microorganism is 0.01 to 0.03.

3. The digested phagocyte according to claim 1 or 2 wherein a density of the phagocyte digested with the foreign microorganism is 1×10⁴ cells/μl to 5×10⁴ cells/μl.

4. The digested phagocyte according to any one of claims 1-3 wherein said foreign microorganism is a gram negative bacterium.

5. The digested phagocyte according to any one of claims 1-3 wherein said foreign microorganism is one or more microorganism selected from the group consisting of Staphylococcus aureus, Staphylococcus epidermidis, Enterococcus faecalis, Pseudomonas aeruginosa, Escherichia coli and Candida albicans, and a mixture thereof.

6. A process for producing a phagocyte digested with a foreign microorganism comprising the steps of:
contacting in vitro a phagocyte with a foreign microorganism; and
isolating the phagocyte.

7. The process according to claim 6 wherein a turbidity of bacterial liquid (O.D.=600 nm) of the foreign microorganism used for in vitro contact between the phagocyte and the foreign microorganism is 0.01 to 0.03.

8. The process according to claim 6 or 7 wherein a density of the phagocyte digested with the foreign microorganism is 1×10⁴ cells/μl to 5×10⁴ cells/μl.

9. The process according to any one of claims 6-8 wherein said foreign microorganism is a gram negative bacterium.

10. The process according to any one of claims 6-8 wherein said foreign microorganism is one or more microorganism selected from the group consisting of Staphylococcus aureus, Staphylococcus epidermidis, Enterococcus faecalis, Pseudomonas aeruginosa, Escherichia coli and Candida albicans, and a mixture thereof.

11. A process for detecting and/or identifying a digested foreign microorganism comprising the steps of:
fixing the phagocyte digested with a foreign microorganism according to any one of claims 1-5;
treating to promote permeability of the cell membrane of the phagocyte;
treating to expose DNA of the foreign microorganism existing in the phagocyte;
in situ hybridizing under a stringent condition between a DNA probe which can detect hybridization and the DNA; and
detecting and/or identifying the digested foreign microorganism by the resulting signal.

12. A process for evaluating a phagocytotic function against a foreign microorganism comprising the steps of:
fixing the phagocyte digested with a foreign microorganism according to any one of claims 1 to 5;
treating to promote permeability of the cell membrane of the phagocyte;
treating to expose DNA of the foreign microorganism existing in the phagocyte;
in situ hybridizing under a stringent condition between a DNA probe which can detect hybridization and the DNA; and
identifying by the resulting signal the phagocytosis and/or killing ability of the phagocyte against the foreign microorganism.

13. The process according to claim 11 or 12 wherein said process includes at least one aspect of:
(1) the density (X cells/ml) of the phagocytes to be fixed is 5×10⁶ cells/ml<X cells/ml<1×10⁸ cells/ml;
(2) in said exposing step of the DNA, lysostafin having the titer of 1 unit/ml to 1,000 unit/ml is used;
(3) in said exposing step of the DNA, lysozyme having the titer of 1,000 unit/ml to 1,000,000 unit/ml is used;
(4) in said exposing step of the DNA, N-acetylmuramidase having the titer of 10 unit/ml to 10,000 unit/ml is used;
(5) in said exposing step of the DNA, zymolase having the titer of 50 unit/ml to 500 unit/ml is used;
(6) in said in situ hybridization step, a surfactant is used;
(7) said DNA probe for detection is one or more DNA probe having the chain length of 350 to 600 base length; and
(8) the concentration of said DNA probe for detection is 0.1 ng/μl to 2.2 ng/μl.

14. The process according to claim 13 wherein one or more enzyme selected from lysostafin, lysozyme, N-acetylmuramidase and zymolase is used in said exposing step of the DNA, with the titer of lysostafin being 10 unit/ml to 100 unit/ml; the titer of lysozyme being 10,000 unit/ml to 100,000 unit/ml; the titer of N-acetylmuramidase being 100 unit/ml to 1,000 unit/ml; and the titer of zymolase being 100 unit/ml to 500 unit/ml.

15. The process according to any one of claims 11 to 14 wherein an enzyme is used in said exposing step of the DNA, and wherein the temperature to allow the reaction of the enzyme is 26° C. to 59° C., with the time period of the reaction of the enzyme being 15 minutes to 120 minutes.

16. The process according to any one of claims 11 to 15 wherein a substance for retaining the morphology of the phagocyte is additionally used in said exposing step of the DNA.

17. The process according to claim 16 wherein said substance is phenylmethylsulfonyl fluoride.

18. The process according to claim 17 wherein the concentration of said phenylmethylsulfonyl fluoride is 10 μmol/l to 10 mmol/l.

19. The process according to any one of claims 16 to 18 wherein said substance is a substance dissolved in dimethylsulfoxide.

20. The process according to claim 19 wherein the concentration of said dimethylsulfoxide is less than 5%.

21. The process according to any one of claims 11 to 20 wherein the DNA and the DNA probe is hybridized in the presence of a surfactant in said in situ hybridization step.

22. The process according to claim 21 wherein said surfactant is an anion surfactant.

23. The process according to claim 22 wherein said anion surfactant is sodium dodecylsulfate.

24. The process according to any one of claims 11 to 23 wherein the temperature to allow the hybridization reaction is 25° C. to 50° C., with the time period of the hybridization reaction being 30 minutes to 900 minutes in said in situ hybridization step.

25. A process for evaluating a phagocytotic function against a foreign microorganism comprising the steps of:
fixing the digested phagocyte according to any one of claims 1 to 5;
staining the phagocyte with a dye; and
identifying the phagocytosis and/or killing ability of the phagocyte against the foreign microorganism by the detection through observation by microscopic examination on cell morphology which is characteristic in cells during or after phagocytosis.

26. A process for evaluating an immune function comprising the steps of:
isolating phagocytes from a subject;
evaluating a function of the phagocytes using the process for evaluating a phagocytotic function according to any one of claims 12 to 25; and
evaluating the immune function of the subject by comparing the evaluation result to that of the function of normal phagocytes.

27. The process according to claim 26 wherein said immune function is a phagocytotic ability of a microorganism by a leukocyte.

28. The process according to claim 27 wherein said immune function is a phagocytotic ability against a microorganism by a leukocyte of a patient who received the radiation exposure or the administration of an anticancer agent.

29. A process for evaluating differentiation efficiency into a phagocyte comprising the steps of:
evaluating a phagocytotic function against a foreign microorganism according to any one of claims 12 to 25; and
evaluating the phagocytotic function in a time dependent manner to identify the alteration.

30. A process of the evaluation for determining an effect of a modulator of phagocytotic function comprising the steps of:
allowing phagocytosis by incubating a suspension of a foreign microorganism and phagocytes in the presence and absence of a phagocytotic function modulator; and
comparing the phagocytotic function in the presence and absence of said phagocytotic function modulator using the process for evaluating a phagocytotic function against a foreign microorganism according to any one of claims 12 to 25.

31. A process for screening a modulator of phagocytotic function comprising the steps of:
allowing phagocytosis by incubating a suspension of a foreign microorganism and phagocytes in the presence and absence of a candidate agent supposed to have a modulatory action toward the phagocytotic function; and
comparing the phagocytotic function in the presence and absence of said agent using the process for evaluating a phagocytotic function against a foreign microorganism according to any one of claims 12 to 25.

32. A clinical testing process comprising the steps of:
obtaining phagocytes from a subject prior to and following the administration of an agent to the subject;
evaluating a function of the phagocyte using the process for evaluating a phagocytotic function according to any one of claims 12 to 25; and
examining a dosage regimen of the agent judging from the effect of the agent determined on the basis of the evaluation result.

33. A performance testing process of a kit for evaluating a phagocytotic function which comprises fixing phagocytes, treating to promote permeability of the cell membranes of the phagocytes, treating to expose the DNA of a foreign microorganism in the phagocytes, in situ hybridize under a stringent condition between the DNA and a DNA probe which can detect hybridization; and evaluating the phagocytotic function by the resulting signal, said kit has;
(1) the foreign microorganism,
(2) at least one or more enzyme(s) selected from the group consisting of lysostafin, lysozyme, N-acetylmuramidase and zymolase used in said exposing step of the DNA, and
(3) one or more DNA probe(s) for detection,
said process is characterized in that the digested phagocyte according to any one of claims 1 to 5 is used.

34. A performance testing process of a kit for detecting and/or identifying a foreing microorganism which comprises obtaining phagocytes from a clinical specimen containing phagocytes derived from a living body, fixing the phagocytes so obtained, treating to promote permeability of the cell membranes of the phagocytes, treating to expose the DNA of the foreign microorganism predicted as existing in the phagocytes, in situ hybridizing under a stringent condition between the DNA and a DNA probe which can detect hybridization, and detecting and/or identifying the foreign microorganism by the resulting signal,
the process is characterized in that the digested phagocyte according to any one of claims 1 to 5 is used.

35. The performance testing process according to claim 33 or 34 wherein said performance test is a sensitivity test, a specificity test or a reproducibility test.

36. The performance testing process according to claim 33 or 34 wherein the digested phagocyte according to any one of claims 1 to 5 is used as a positive control.

37. The process according to any one of claims 11 to 36 wherein the process further comprises a step prior to said fixing step to put the digested phagocyte onto a solid support which is a slide glass coated with 3-aminopropyltriethoxysilane.

38. The process according to any one of claims 11 to 37 wherein a dye for clarifying the contrast between the signal and the cell is used upon the detection of said signal.

39. The process according to any one of claims 11 to 38 wherein said phagocyte is from blood.

40. A kit for evaluating a phagocytotic function by fixing the digested phagocytes according to any one of claims 1 to 5, treating to promote permeability of the cell membranes of the phagocytes, treating to expose DNA of the foreign microorganism in the phagocytes, in situ hybridizing under a stringent condition between the DNA and a DNA probe which can detect hybridization; and evaluating the phagocytotic function by the resulting signal, wherein said kit has;
(1) the foreign microorganism,
(2) at least one or more enzyme(s) selected from the group consisting of lysostafin, lysozyme, N-acetylmuramidase and zymolase used in said exposing step of the DNA, and
(3) one or more DNA probe(s) for detection.
